(19) United States
(12) Patent Application Publication
(10) Pub. No.: US 2026/0102377 A1
(43) Pub. Date: Apr. 16, 2026

(54) COMPOSITIONS AND METHODS FOR TREATING MYASTHENIA GRAVIS

(71) Applicant: NMD PHARMA A/S, Århus N (DK)

(72) Inventors: Thomas Holm PEDERSEN, Aarhus N (DK); John Blundell HUTCHISON, Aarhus N (DK); Ole Baekgaard NIELSEN, Aarhus N (DK); Lars J.S. KNUTSEN, Aarhus N (DK); Lars NORDHOLM, Aarhus N (DK); Nicholas Michael KELLY, Aarhus N (DK); Eva Rose CHIN, Aarhus N (DK); Klaus Gjervig JENSEN, Aarhus N (DK); Peter FLAGSTAD, Aarhus N (DK); Jane Mary BOLD, Aarhus N (DK); Thomas Skjaerlund GRØNNEBAEK, Aarhus N (DK); Jorge Armando Quiroz BASTIAS, Aarhus N (DK)

(73) Assignee: NMD PHARMA A/S, Århus N (DK)

(21) Appl. No.: 19/116,795

(22) PCT Filed: Sep. 28, 2023
(86) PCT No.: PCT/EP2023/076934

§ 371 Date: Mar. 28, 2025

(30) Foreign Application Priority Data
Sep. 30, 2022 (EP) 22199117.7
Mar. 2, 2023 (EP) 23159662.8

(51) Int. Cl. A61K 31/42; A61K 47/12; A61K 47/36; A61K 47/38; A61P 21/04

(52) U.S. Cl. CPC A61K 31/42; A61K 47/12; A61K 47/36; A61K 47/38; A61P 21/04

(57) ABSTRACT

Disclosed herein are methods of treatment for myasthenia gravis (MG) that involve administering (2S)-2-[4-bromo-2-(1,2-oxazol-3-yl)phenoxy]propanoic acid, or a pharmaceutical composition thereof, to a patient suffering from symptoms of MG. Pharmaceutical compositions and kits-to-parts including (2S)-2-[4-bromo-2-(1,2-oxazol-3-yl)phenoxy]propanoic acid are also disclosed.

### TECHNICAL FIELD

The present disclosure relates to (2S)-2-[4-bromo-2-(1,2-oxazol-3-yl)phenoxy]propanoic acid for use in the treatment of myasthenia gravis (MG), its pharmaceutical composition for use in the treatment of MG and methods of treatment thereof.

### BACKGROUND

Myasthenia gravis (MG) is an autoimmune disease of the neuromuscular junction synapse (NMJ) characterized by weakness and fatigability of voluntary skeletal muscles that worsens with continued muscle work and improves with resting of the involved muscle(s) (Borges et al., Front. Immunol. 2020, 11:707). For MG, the distribution of weakness is distinctive often involving extraocular muscles. MG may initially begin with ocular muscle weakness affecting eye and eyelid movement, referred to as ocular MG (oMG). In more severe cases (generalized MG), the pontine- and bulbar-innervated muscles and the respiratory muscles are commonly also affected. Least frequently involved are the extremity muscles.

MG may cause life-threatening respiratory failure, referred to as myasthenic crisis. About 15% to 20% of subjects will experience a myasthenic crisis during the course of their disease, 75% within 2 years of diagnosis, requiring hospitalization and ventilatory support.

MG has a prevalence in 14-20 people per 100,000 in the U.S., affecting roughly 60,000 Americans. It affects males and females in equal ratio, although the incidence in females peaks in the 3rd decade as compared to males in whom the peak age at onset is in the 6th or 7th decade. Mortality from MG is approximately 4%, mostly due to respiratory failure.

MG is generally caused by autoantibodies that target the neuromuscular junction. In generalized MG, about 90% of patients have circulating antibodies (Abs) that target the nicotinic acetylcholine receptor (AChR), which is a postsynaptic neurotransmitter receptor. Loss of AChR function can result in compromised neuromuscular transmission that can lead to failing muscle fibre activation and eventually muscle weakness and excessive fatigability. At the neuromuscular junction, the transmission of the nerve action potential to the muscle membrane involves flow of both excitatory and inhibitory currents. Excitation of muscle requires that the excitatory current outweighs inhibitory current flow. In myasthenia gravis the excitatory current flow is reduced due to the loss of AChR.

Other commonly detected antibodies in patients suffering from MG are antibodies that target the muscle-specific kinase (MuSK) protein. This form of MG is known as MuSK-MG. (Borges et al., Front. Immunol. 2020, 11:707). Although MuSK-MG is also an Ab-mediated disease, inflammatory damage to the NMJ does not occur. In fact, the majority of the Abs are of the IgG4 immunoglobulin subclass, which is characterized partly by the inability to activate complement or bind to Fc receptors. The proposed mode of action of these auto-Abs is blockade of the normal function of MuSK.

While there is no cure for MG, current available treatments for generalised myasthenia gravis aim to modulate neuromuscular transmission, inhibit the production or effects of pathogenic antibodies, inhibition of the cleavage of complement component 5, or inhibit inflammatory cytokines. The current standard of care usually combines cholinesterase inhibitors (most commonly pyridostigmine), corticosteroids (typically prednisone) and immunosuppressive drugs (most commonly azathioprine, cyclosporine, and mycophenolate mofetil), the majority of subjects with MG have their disease reasonably well controlled. Some patients though are refractory or intolerant to Standard of Care treatments and would benefit from new treatment options.

Many of the standard treatments of AChR-MG are of limited effectiveness in MuSK-MG, including thymectomy and cholinesterase inhibitors. Therefore, current treatment involves immunosuppression, primarily by corticosteroids or B cell depletion agents.

Skeletal muscle specific CIC-1 chloride ion channels carry the inhibitory currents that counteract neuromuscular transmission. Inhibition of CIC-1 reduces the inhibitory current and thereby increases muscle membrane excitability and enhances neuromuscular transmission. This was shown to lead to recovery of muscle function in nonclinical models of several neuromuscular diseases (Pedersen et al., Acta Physiol. 2021, 1-14).

Accordingly, the CIC-1 ion channel is emerging as a target for potential drugs, although its potential has been largely unrealized. U.S. Pat. No. 10,385,028 discloses the synthesis of compounds which have been designed to inhibit the action of the CIC-1 ion channel to treat neuromuscular disorders.

One of the compounds discussed in U.S. Pat. No. 10,385,028 is (2S)-2-[4-bromo-2-(1,2-oxazol-3-yl)phenoxy]propanoic acid, hereinafter NMD670. The chemical structure of NMD670 is provided below.

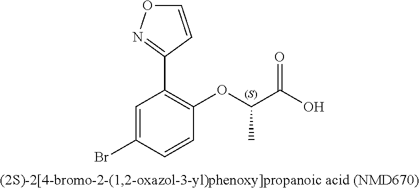

Though U.S. Pat. No. 10,385,028 discloses a set of compounds which can inhibit the CIC-1 ion channel to treat neuromuscular disorders, there is no discussion into how to design treatment methods to where these compounds can effectively alleviate the wide range of symptoms associated with myasthenia gravis that fluctuate from the day-to-day. WO2020/254554, herein incorporated by reference, discloses methods for manufacturing NMD670.

Accordingly, there is a need for safe and efficacious therapies to improve muscle function in patients with all forms of MG, due to the limited effect of and/or side effects caused by existing therapies.

### SUMMARY

The present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein the composition is for administration at a therapeutic dose of 100 to 1500 mg of (2S)-2-[4-bromo-2-(1,2-oxazol-3-yl)phenoxy]propanoic acid.

The present disclosure further relates to a composition, formulated as a solid dosage form, comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition comprises 50 to 400 mg (2S)-2-[4-bromo-2-(1,2-oxazol-3-yl)phenoxy]propanoic acid.

The present disclosure further relates to methods for improving the Quantitative Myasthenia Gravis total score or improving right hand grip strength in a subject suffering from myasthenia gravis comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.

The present disclosure further relates to kit-of-parts or a composition comprising (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor for use in a method for treatment of myasthenia gravis in a subject.

### DESCRIPTION OF DRAWINGS

FIG. 1 depicts the effects of NMD670 on membrane conductance (Gₘ) in rat soleus muscle from healthy (n=20) and myasthenia gravis (EAMG) rats (n=12) as the average Gₘ measured in a muscle before (black circles or open squares respectively) and after addition of 20 μM NMD670 (black triangles or open inverted triangles respectively). Black overlay bars are averages±SEM of each group. Addition of NMD670 statistically significantly reduced Gₘ in both healthy and EAMG animals.

FIG. 2 depicts the effect of CIC-1 inhibition on skeletal muscle fibre excitability as evaluated from rheobase current in skeletal muscle fibres from healthy and myasthenia gravis (EAMG) rats. Rheobase current (nA) in soleus muscle fibres from healthy (n=20) and EAMG rats (n=12) as the average rheobase current measured before (black circles or open squares respectively) and after addition of 20 μM NMD670 (black triangles or open inverted triangles respectively). Black overlay bars show averages±SEM of groups. Addition of NMD670 significantly reduced rheobase current in muscle fibres from both groups of rats.

FIG. 3 depicts the endplate potential (EPP) amplitude in healthy rats (left trace), untreated myasthenia gravis (EAMG) rats (middle trace) and EAMG rats after addition of 20 μM of NMD670 (right trace). Compared to EPP in healthy rats, EPP were markedly reduced in muscle fibres from EAMG rats but could be restored after addition of NMD670.

FIG. 4 depicts the average endplate potential (EPP) amplitude in 68 muscle fibres from myasthenia gravis (EAMG) rats without addition of a CIC-1 inhibitor (black diamond) and 82 fibres in the presence of 20 μM NMD670 (open circle) during 12 Hz stimulation for 30 stimulations. With CIC-1 inhibition by NMD670, the EPP increased by more than 35% throughout a 12 Hz train of stimulation.

FIG. 5 depicts the action potentials at 30 Hz stimulation in muscle fibres from healthy rats (left trace), untreated myasthenia gravis (EAMG) rats (middle trace) and EAMG rats after addition of 20 μM of NMD670 (right trace). Sustained action potential firing was compromised in nerve-muscle preparations from untreated EAMG rats (middle trace) compared to healthy rats (left trace), but the success of repeated action potential excitations was improved markedly after addition of 20 μM NMD670 (right trace).

FIG. 6 depicts the average action potential generation success (%) of attempted stimulations at 30 Hz stimulation pre and post treatment of 20 μM NMD670 (n=110 muscle fibres in both groups). After addition of 20 μM NMD670, the success of repeated action potential excitations was improved markedly.

FIG. 7 depicts the force from isolated soleus muscle nerve-stimulated at 60 Hz from healthy rats (left trace), untreated myasthenia gravis (EAMG) rats (middle trace) and EAMG rats after addition of 20 μM of NMD670 (right trace). Compared to force in healthy rats, force was markedly reduced in muscle fibres from EAMG rats but could be restored after addition of NMD670.

FIG. 8 depicts the average force in isolated nerve-stimulated soleus (n=14), EDL (n=14) and diaphragm (n=12) muscles from EAMG animals treated with 20 μM NMD670 compared to pre-treated muscles (value set to 100). Addition of NMD670 restored force generation to levels that were close to the observations in muscles from healthy animals.

FIG. 9 depicts the nerve-stimulated force (lower panel) and EMG amplitude (upper panel) in tricpes surae muscle from an aged-matched healthy rat (left traces) and an myasthenia gravis (EAMG) rat with EAMG score 2, stimulated via the sciatic nerve at 80 Hz for 1 second, before (middle traces) and 20 minutes after administration of 40 mg per kg of bodyweight NMD670 (right traces). Administration of NMD670 in EAMG animals caused rapid restoration of force and EMG amplitude.

FIG. 10 depicts average muscle force (mean±SEM) from myasthenia gravis (EAMG) rats (as exemplified in FIG. 8) before (white) and after (grey) receiving NMD670 per oral (from 2 to 120 mg/kg), relative to muscle force from a healthy age matched rat. The figure shows that administration of NMD670 in EAMG rats caused rapid and dose-dependent restoration of muscle force. The number in the column indicates the number of rats per dose group.

FIG. 11 depicts the grip strength increase (45 minutes after dosing) from EAMG rats receiving vehicle (black, n=35), 0.375 mg per kg of bodyweight Mestinon (checked pattern, n=10), 20 mg per kg of bodyweight NMD670 (diagonal lines, n=17) or a combination of 0.375 mg per kg of bodyweight Mestinon and 20 mg per kg of bodyweight NMD670 dosed simultaneously (horizontal lines, n=5). Grip strength significantly increased after administration of NMD670, or a combination of NMD670 and Mestinon. Vehicle treatment did not affect grip strength.

FIG. 12 depicts the grip strength relative to bodyweight during 14-day chronic dosing in EAMG rats receiving vehicle (filled black circles) or 20 mg/kg NMD670 twice daily (open circles). Rats that received NMD670 had increased grip strength over vehicle group throughout the period of treatment.

FIG. 13 depicts the rotarod performance relative to bodyweight during 14-day chronic dosing in EAMG rats receiving (filled black circles) or 20 mg/kg NMD670 twice daily (open circles). Rats that received NMD670 had higher endurance on rotarod (longer latency to fall) over vehicle group throughout the period of treatment.

FIG. 14 depicts the bodyweight relative to the individual bodyweight at day 0 of study during 14-day chronic dosing in EAMG animals receiving vehicle (filled back circles) or 40 mg/kg BID NMD670 (open cirles). Treated animals showed an attenuation in loss of body weight (versus vehicle) during the study.

FIG. 15 depicts the number of adverse events recorded during the single-ascending dose study. Row 4 describes the total incidence of all adverse events (AEs) for that dose of NMD670 and the number of subjects that reported that AE are in parenthesis. Row 5 describes the total incidence of severe adverse events (SAEs) The lower section of the table describes the incidence of the most common adverse events (recorded in more than 1 subject). The number of subjects that reported that AE are in parenthesis.

FIG. 16 depicts the study design of a double-blinded, placebo-controlled, three-way cross-over comparison of two single oral doses of NMD670 in men and women with stable symptomatic myasthenia gravis.

FIG. 17 depicts the schedule of activities during Part C of the Phase IIA clinical trial. QMG: quantitative myasthenia gravis scale; RNS: repetitive nerve stimulation; MVRC: muscle velocity recovery cycles; RoVEMP: repetitive ocular vestibular myogenic potentials. Timepoints indicated in the schedule are approximate timepoints.

FIG. 18 depicts the study diagram of a prophetic Phase 2b clinical trial. BID=bis in diem/twice a day; N=number of participants.

FIGS. 19A and 19B depict the schedule of activities of a prophetic Phase 2b clinical trial. Notes: If possible, assessments should be conducted at the same time at the different visits and in the order presented in the table, unless specified otherwise. Abbreviations: AE=adverse event; C-SSRS=Columbia-Suicide Severity Rating Scale; ECG=electrocardiogram; EOT=end of treatment; FU=follow-up; HIV=human immunodeficiency virus; IRT=interactive response technology; Myasthenia Gravis Activities of Daily Living; MGC=Myasthenia Gravis Composite; MG-QOL15r=Myasthenia Gravis Quality of Life 15 Scale Item revised QMG=Quantitative Myasthenia Gravis Score; Neuro-QoL; PK=pharmacokinetics; SAE=serious adverse event; WOCBP=woman of childbearing potential

### DEFINITIONS

All publications, patent applications, patents and other references mentioned herein are incorporated by reference in their entirety.

The nomenclature used in the present application is based on IUPAC systematic nomenclature, unless indicated otherwise.

The term “patient” or “subject” refers to a human (such as a male or female human) who has been diagnosed with MG. The Myasthenia Gravis Foundation of America Classification (MGFA) clinical classification (Barnett et al., Neurol Clin. 2018, 36(2):339-353) may be used to diagnose and grade patients having MG.

The term “quantitative myasthenia gravis total score” (QMG total score) refers to a standardised test (Barohn et al., Ann N Y Acad Sci 1998, 841:769-72) of 13 items (see Table 1) that are used in the context of a clinical trial to measure muscle strength, endurance or fatigability. The items measure the following symptoms and signs: ptosis, diplopia, double vision, swallowing, speech (onset of dysarthria), percent predicted forced vital capacity, grip strength (2 items), arm muscle endurance (2 items), leg muscle endurance (2 items), and head lifting endurance. All items are scored on a scale of 0 to 3, and total scores range from 0 to 39; higher scores indicate greater disease severity.

The term “improvement” refers to a lessening of a patient's myasthenia gravis (MG) symptoms when the patient is administered a composition described herein compared to a patient's MG symptoms when the patient is administered a placebo. The term “improvement” may also refer to lessening of a group of patients' MG symptoms after the group of patients has been administered a composition as described herein, e.g. as evaluated based on comparative test scores between the patient group being administered the composition as described herein with a control group receiving e.g. placebo. An improvement in a patient's MG symptoms can be determined for example using the quantitative myasthenia gravis (QMG) total score (where a reduction in the QMG total score means that a patient's symptoms have improved); an improvement in hand grip strength (where an increase in force measured by the dynamometer means that a patient's could pull a larger weight); an increase in compound muscle action potentials; a recovery of muscle decrement; a decrease in the MG activities of daily living profile (MG-ADL) score, an increase in muscle strength, a decrease in the Myasthenia Gravis Composite (MGC) scale, a decrease in the Myasthenia Gravis Quality of Life 15 (MG-QOL15) score, an increase in health state when determined using the EQ-5D scale, a reduction in jitter; a reduction in blocking; a decrease in the Individualised Neuromuscular Quality of Life score; a decrease in the Fatigue Severity Scale score and/or an improvement in pulmonary function. In an exemplary embodiment, an improvement in MG symptoms includes a reduction in the QMG total score.

The term “jitter” refers to the variability in the arrival time of action potentials to the recording electrode between consecutive electrical discharges when measuring neuromuscular function using single fiber electromyography (sfEMG).

The term “blocking” refers to complete NMJ transmission failure of action potentials to the recording electrode between consecutive electrical discharges when measuring neuromuscular function using sfEMG.

The term “placebo” refers to a dosage form possessing no therapeutic activity.

The term “active pharmaceutical ingredient” (or “API”) denotes the compound or molecule in a pharmaceutical composition that has a particular biological activity.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” refers to a preparation which is in such form as to permit the biological activity of an active ingredient contained therein to be effective, and which contains no additional components which are unacceptably toxic to a subject to which the composition would be administered.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

A “pharmaceutically acceptable carrier” refers to an ingredient in a pharmaceutical composition, other than an active ingredient, which is nontoxic to a subject. A pharmaceutically acceptable carrier includes, but is not limited to, a buffer or acidifier, excipient, stabilizer, or preservative.

The term “solid dosage form releases” means the amount of compound that is released or dissolved into solution after a specified period of time when using a United States Pharmacopeia (USP) type 2 dissolution apparatus, paddle speed of 75 rpm, at a temperature of 37° C.±0.5° C. in 900 mL of pH 6.8 phosphate/citric acid buffer as described in Example 10.

The term “Cₘₐₓ” (expressed in units of ng/mL) means maximum observed plasma concentration of NMD670. The term “mean Cₘₐₓ” means the arithmetic mean of the individual Cₘₐₓ values.

The term “Tₘₐₓ” (expressed in units of hours, or as a median number of hours for Tₘₐₓ in the study population) means the observed time to reach Cₘₐₓ following drug administration; if it occurs at more than one time point Tₘₐₓ is defined as the first time point with this value.

The term “dose” means the dose of NMD670 as free acid that was given to the subject. In addition, the term “dose” may be inclusive of NMD670 in combination with a pharmaceutically acceptable salt.

The term “therapeutically effective dose” as used herein refers to the amount of NMD670 required to cause a therapeutic response in a subject. The terms “therapeutically effective dose” and “therapeutic dose” are used interchangeably herein.

The composition comprising the (therapeutic) dose may be administered in one or more unit dosage forms. As used herein, “unit dosage forms” refers to physically discrete units suitable for human and animal subjects. Each unit dosage includes a predetermined quantity of the therapeutically active compound, in association with, when required, a pharmaceutical carrier, vehicle or diluent. Examples of unit dosage forms include tablets, capsules, pills, powders, granules, sterile parenteral solutions or suspensions, ampoules and syringes, and oral solutions or suspensions, and oil-water emulsions. Unit dosage forms can be individually packaged as is known in the art, such as in blister packs. Unit dosage forms can be administered in fractions or multiples thereof.

The term “T_{1/2}” (expressed in units of hours) means the terminal elimination half-life of NMD670 in plasma.

The term “AUC_{0-infinity}” (expressed in units of h·ng/mL) means the cumulative area under the plasma time concentration curve (AUC) calculated using the trapezoidal method from time 0 to infinity after a single dose of NMD670. The term “mean AUC_{0-infinity}” means the arithmetic mean of the individual AUC_{0-infinity} values.

The term “AUC₀₋₂₄ₕₒᵤᵣₛ” (expressed in units of h·ng/mL) means the cumulative area under the plasma time concentration curve (AUC) calculated using the trapezoidal method from time 0 to 24 hours after a single dose of NMD670. The term “mean AUC₀₋₂₄ₕₒᵤᵣₛ” means the arithmetic mean of the individual AUC₀₋₂₄ₕₒᵤᵣₛ values.

As used within the following disclosure, the term “NMD670” refers to (2S)-2-[4-bromo-2-(1,2-oxazol-3-yl)phenoxy]propanoic acid, in addition to any pharmaceutically acceptable salt, hydrate, polymorph, tautomer, or solvate thereof. (2S)-2-[4-bromo-2-(1,2-oxazol-3-yl)phenoxy]propanoic acid, according to the present disclosure, refers to a compound of formula (I) below, CAS Number 2354321-33-6.

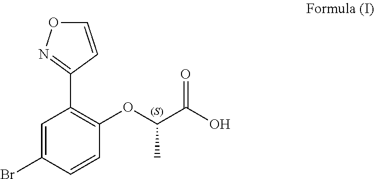

All publications, patent applications, patents and other references mentioned herein are incorporated by reference in their entirety.

### DETAILED DESCRIPTION

Myasthenia gravis is either an autoimmune or congenital neuromuscular disorder that leads to fluctuating muscle weakness and fatigue. In the most common cases, muscle weakness is caused by circulating antibodies that block ACh receptors at the postsynaptic neuromuscular junction, inhibiting the excitatory effects of the neurotransmitter ACh on nicotinic ACh-receptors at neuromuscular junctions.

NMD670 is a compound that was designed to inhibit the CIC-1 ion channel to treat neuromuscular disorders, one of which being myasthenia gravis. Inhibition of the CIC-1 ion channel can lower the occurrence of inhibitory currents flowing in the NMJ region of muscle fibres, thereby prolonging the effects of excitatory currents flowing in the NMJ region. This prolongation can lead to improved muscle contraction and control in patients suffering from myasthenia gravis.

The inventors of the present disclosure, after performing various non-clinical and clinical trial experiments, have discovered treatment regimens and compositions that can allow NMD670 to treat the variable symptoms of myasthenia gravis, thereby granting patients suffering from myasthenia gravis an improved quality of life. Various aspects and embodiments of these treatment methods and compositions will be described as follows. These various aspects and embodiments, however, may be embodied in many different forms. Thus, the present disclosure should not be construed as being limited to these embodiments; rather, these embodiments are provided so that this disclosure will be thorough and complete, and will fully convey the scope of the technology described herein to those skilled in the art. The various non-clinical and clinical trial experiments leading to the discovery of the treatment methods described herein are detailed below.

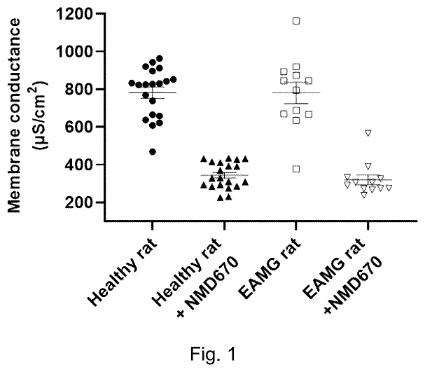

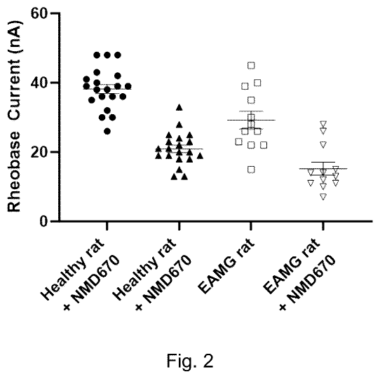

To evaluate whether CIC-1 inhibition could enhance neuromuscular transmission and restore muscle function in conditions of disease-mediated NMJ transmission failure, the effect of CIC-1 inhibition was initially determined from the level of the synapse to that of intact skeletal muscle. This was done with acute and chronic dosing, in an active immunization MG rat model (EAMG) that presents with severe symptoms of muscle weakness and excessive fatigue (Losen et al., Exp. Neurol. 2015, 270:18-28). The ability of NMD670 to inhibit CIC-1 was first confirmed in muscle fibres from healthy rats using electrophysiological techniques with three intracellular microelectrodes (FIG. 1) (Riisager et al. J Physiol. 2014, 592(20):4417-29). The same approach was used to confirm CIC-1 inhibition by NMD670 in muscles from the EAMG rats. It confirmed that neuromuscular disease did not affect CIC-1 function per se nor the ability of NMD670 to inhibit the CIC-1 channel. Inhibition of CIC-1 in the muscle fibres from MG rats increased the excitability of the muscle fibres as observed by a reduction in the rheobase current required to trigger a muscle fibre action potential (FIG. 2).

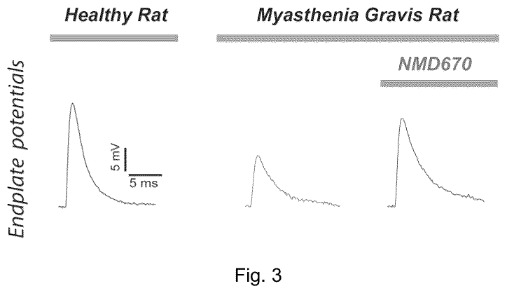

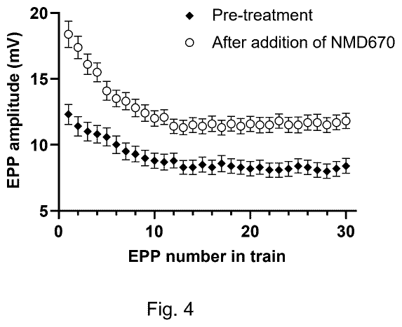

To determine the effect of CIC-1 inhibition at the single synapse level, two series of experiments were conducted using isolated nerve-muscle preparations from severely affected EAMG rats. In both series, the motor nerve was stimulated electrically, and intracellular electrodes inserted at the NMJ recorded the membrane potential in the muscle fibre in response to the nerve-stimulation. In the first series of experiments, the effect of CIC-1 inhibition on EPP was determined. In these experiments muscle fibre action potential excitation was prevented by selectively blocking the skeletal muscle specific voltage gated Na⁺ channels with μ-conotoxin. Similar to findings in MG patients (Elmqvist, et al. J. Physiol. 1965, 178:505-529), EPP amplitudes were markedly reduced in muscle fibres from EAMG rats versus healthy animals (FIG. 3). With CIC-1 inhibition by NMD670, the EPP amplitudes increased by more than 35% throughout a 12 Hz train of stimulation (FIG. 4).

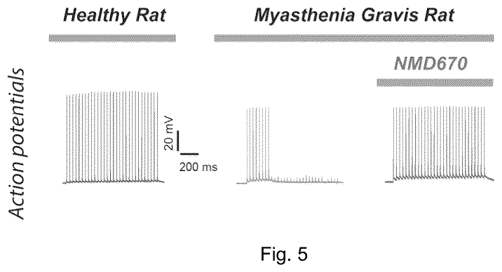

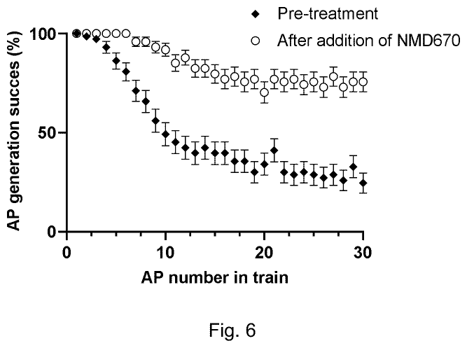

In the second series of experiments at the single synapse level, it was explored whether increased EPP amplitude with CIC-1 inhibition was associated with restored NMJ transmission in EAMG muscle. In these experiments, action potentials were recorded during short trains of nerve stimulation before and after CIC-1 inhibition. As shown in FIG. 5, sustained action potential firing was compromised in nerve-muscle preparations from untreated EAMG animals (FIG. 5, middle trace) compared to healthy animals (FIG. 5, left trace). After addition of 20 μM NMD670, the success of repeated action potential excitations was improved markedly (FIG. 5, right trace and FIG. 6). Taken together, the restored EPP amplitude (FIG. 4) and improved action potential firing (FIG. 6) confirmed that CIC-1 inhibition enhances neuromuscular transmission in an EAMG model.

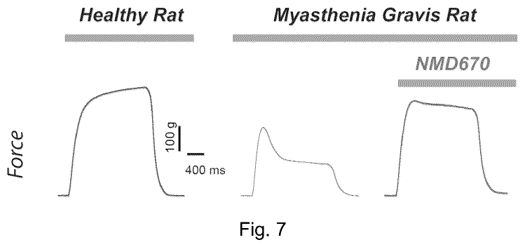

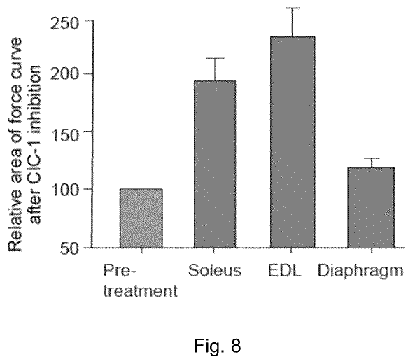

Whether the enhanced synaptic strength following CIC-1 inhibition in muscles from EAMG rats resulted in restored muscle function was then determined using both ex vivo and in vivo approaches. Isolated nerve-muscle preparations were mounted in tissue baths and force production was triggered by nerve stimulation. In contrast to well-maintained force production in muscles from age-matched healthy rats, muscles from EAMG animals were unable to sustain force generation during short periods of nerve stimulation (FIG. 7). Such excessive fatigue during stimulation was prominent in distal hindlimb muscles but also observed in diaphragm muscle. In all muscles, CIC-1 inhibition using 20 μM NMD670 restored force generation (FIG. 8) to levels that were close to the observations in muscles from healthy animals.

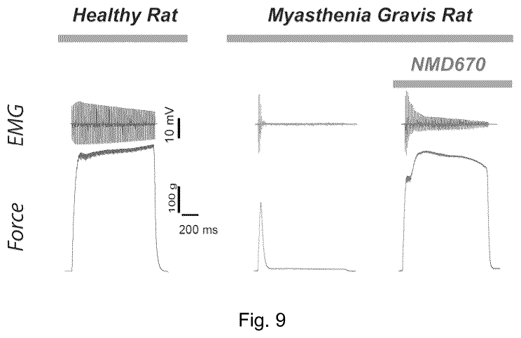

The effect of CIC-1 inhibition on muscle force was next assessed in vivo in EAMG rats with clear symptoms of MG. Rats were anesthetized, and concurrent recordings of electromyography (EMG) and force were obtained from triceps surae muscle during stimulation of the sciatic nerve. Measurements were made before and after oral administration of NMD670, and blood samples were obtained to measure plasma concentration of NMD670 and enable determination of the pharmacokinetic/pharmacodynamic relationship. Similar to observations in isolated preparations, both EMG amplitude and nerve-stimulated muscle force were greatly depressed (FIG. 9, middle traces) in the EAMG compared with healthy animals (FIG. 9, left traces).

Administration of NMD670 in EAMG animals caused rapid and dose-dependent restoration of both EMG and force (FIG. 9, right traces).

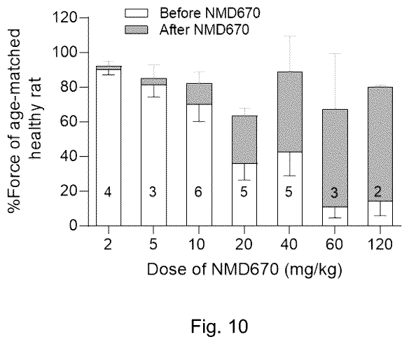

FIG. 10 depicts average muscle force (mean±SEM) from myasthenia gravis (EAMG) rats before (white) and after (grey) receiving NMD670 per oral (from 2 to 120 mg/kg), relative to muscle force from a healthy age matched rat. The figure shows that administration of NMD670 in EAMG rats caused rapid and dose-dependent restoration of muscle force.

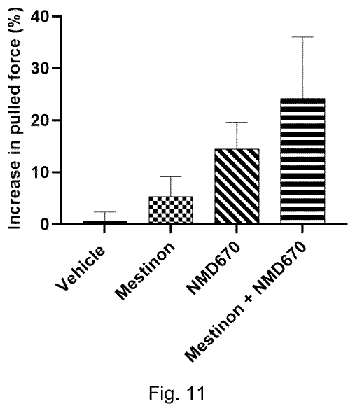

The next series of experiments were conducted with conscious EAMG rats to determine whether enhanced neuromuscular transmission and restored muscle force with CIC-1 inhibition would also increase strength during voluntary movement. Initially the effect of a single administration of NMD670 on grip strength of severely affected EAMG rats was determined. The experiments were conducted in a blinded manner and Mestinon, an acetylcholine esterase inhibitor, was used as a positive control. As shown in FIG. 11, grip strength increased by 15±5% after NMD670 administration and 5±4% with Mestinon. As expected from the different mechanism of action for enhancing neuromuscular transmission, the two approaches were additive (24±12%). Vehicle treatment did not affect grip strength (1±2%). Taken together, these data show that single administration of NMD670 enhanced neuromuscular transmission and restored skeletal muscle function in EAMG rats.

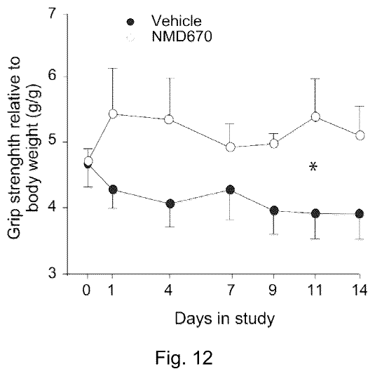

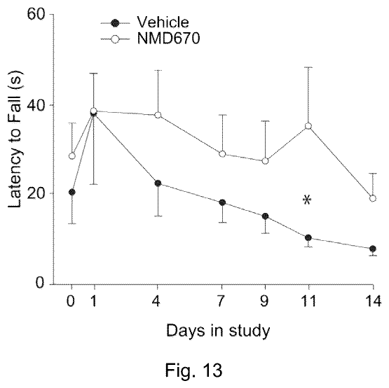

MG is a chronic disease that requires life-long treatment. Given that CIC-1 inhibition has not previously been assessed for treating MG, it was unknown how chronic administration of NMD670 and suppression of CIC-1 function would affect muscle function. For this reason, a 14-day efficacy study was performed in EAMG rats displaying severe symptoms. The effect of twice daily dosing of NMD670 (20 mg/kg) or vehicle was evaluated on bodyweight, grip strength and endurance (rotarod). The study was blinded, and all rats had confirmed presence of antibodies against acetylcholine receptors. Blood sampling was conducted during the 14 days of study and muscle biopsies were taken at study termination to confirm correct dosing between study groups. Rats that received NMD670 had increased grip strength (FIG. 12) and higher endurance on rotarod (FIG. 13) throughout the period of treatment.

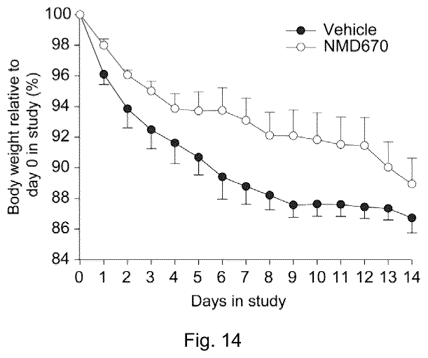

Treated animals also showed an attenuation in loss of body weight (versus vehicle) during the study (FIG. 14). In both groups, early terminations were required when body weight declined below 80% of maximum body weight prior to disease induction. In the NMD670 treated group, 6 of the 8 treated rats completed the study while only 3 out of 8 vehicle treated rats completed the study. These findings thus support the notion that prolonged CIC-1 inhibition improves muscle function chronically in EAMG rats and this improves overall health status.

The effect of CIC-1 inhibition on treating MG was further assessed in a clinical trial. Specifically, a phase I/IIA clinical trial was carried out to initially determine the safety, pharmacokinetics (PK) and pharmacodynamics (PD) of single-ascending doses (SAD) and multiple-ascending doses (MAD) of NMD670 in healthy male and female subjects and the effect of CIC-1 inhibition on treating MG.

Part A1 tested single doses of NMD670 in a double-blind, randomised, placebo-controlled, partial crossover and dose-escalating design in healthy male subjects. A total of nine dose levels were investigated in three cohorts of subjects. Each cohort consisted of nine subjects, each subject had three study sessions. Each subject received escalating doses of NMD670 on two occasions and placebo on one occasion, the order will be randomized in a cross-over fashion. Each dose level was randomized in a 6:3 ratio (active vs. placebo).

Dose escalation was stopped after an adverse event of myotonia of moderate intensity observed in one subject administered 1600 mg of NMD670 at dose level 7 (spontaneously and fully resolved within hours). Due to this temporary halt and partial unblinding of the study during dose level 7, a new randomization was necessary. After unblinding three subjects in dose level 7, the original randomization for dose 8 and 9 was changed in order to keep the study blinded. As subjects of cohort 3 only had 2 occasions left, the randomisation of the original design (3-way cross-over) was not possible without compromising the ratio of active and placebo treated subjects, and therefore the within-subject comparison which is important for the evaluation of PD markers. Therefore, for the remaining 2 doses, a full cross-over design investigating one previously tested dose level was used. The 9 subjects in cohort 3 were randomized to receive the study drug in level 8 and placebo in level 9, or vice versa. To determine the effect of food on the exposure of single oral doses of NMD670, dose level 5 was administered in both the fasted and fed condition. Subjects who received dose level 5 returned for a fourth visit in which they received dose level 5 (or matching placebo) in the fed condition, in the same randomization as the chosen dose level in the fasted condition.

Part A2 of the study investigated the safety, tolerability and pharmacokinetics of NMD670 in 8 healthy female subjects of non-childbearing potential, in a randomized, double-blind, placebo-controlled single dose administration of NMD670. Subjects received 800 mg NMD670. Subjects were randomized in a 6:2 ratio (active vs. placebo).

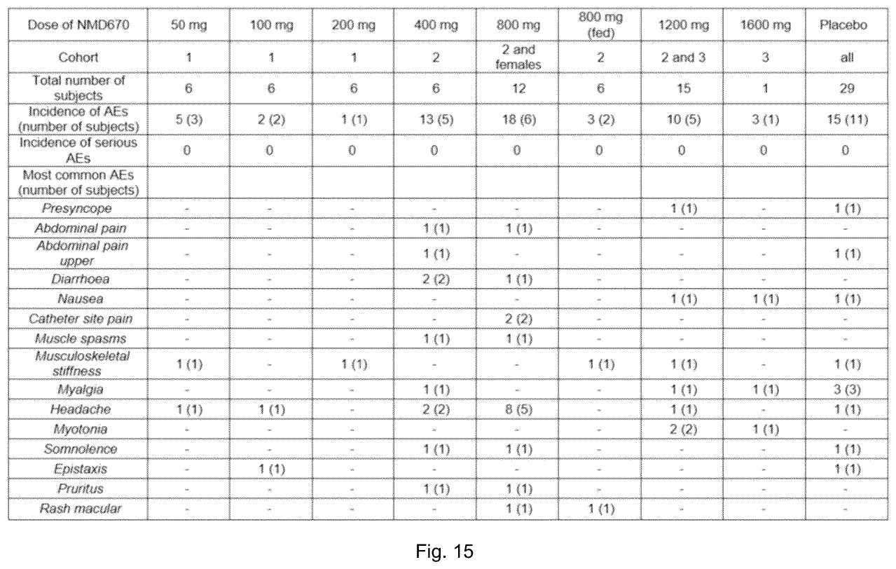

No serious or severe adverse effects were reported. There were no meaningful relationships between increases in NMD670 dose and the incidence of participants with AEs (FIG. 15). A total of 70 AEs were reported, of which 47 (67%) were at least possibly drug-related. Most common AEs reported (in >1 subject) are listed in Table 15. There were no relationships between increases in dose and the incidence of these individual AEs following administration of a single dose of NMD670, except for transient myotonia which was reported at the highest dose levels tested (1200 mg and 1600 mg). Most AEs were mild, except for one AE of myotonia (1600 mg), and tooth extraction (unrelated, 50 mg), which were moderate in intensity. There were no subject discontinuations.

The phase IIA part of the clinical trial was double-blinded, placebo-controlled and included a three-way cross-over comparison of two single oral doses of NMD670 in men and women with stable symptomatic myasthenia gravis (class I, II, III or IVa on the Myasthenia Gravis Foundation of America Classification scale). 12 patients diagnosed with MG were enrolled in this clinical trial.

The Myasthenia Gravis Foundation of America Classification (MGFA) clinical classification is derived from the Osserman score, developed in the 1950s (Osserman et al, AMA Arch Intern Med, 1958, 102(1):72-81), which was one of the first classifications systems in MG. The MGFA classification is aimed at separating patients in groups based on disease severity and the localization of the symptoms, and it does not have an evaluative purpose. The MGFA classes are as follows (Barnett et al, Neurol Clin. 2018, 36(2):339-353):

| | |
|---|---|
| Class I | Any ocular muscle weakness; may have weakness of eye closure. All |
| | other muscle strength is normal. |
| Class II | Mild weakness affecting muscles other than ocular muscles; may also |
| | have ocular muscle weakness of any severity. |
| IIa | Predominantly affecting limb, axial muscles, or both. May also have |
| | lesser involvement of oropharyngeal muscles. |
| IIb | Predominantly affecting oropharyngeal, respiratory muscles, or both. |
| | May also have lesser or equal involvement of limb, axial muscles, or |
| | both. |
| Class III | Moderate weakness affecting muscles other than ocular muscles; may |
| | also have ocular muscle weakness of any severity. |
| IIIa | Predominantly affecting limb, axial muscles, or both. May also have |
| | lesser involvement of oropharyngeal muscles. |
| IIIb | Predominantly affecting oropharyngeal, respiratory muscles, or both. |
| | May also have lesser or equal involvement of limb, axial muscles, or |
| | both. |
| Class IV | Severe weakness affecting muscles other than ocular muscles; may also |
| | have ocular muscle weakness of any severity. |
| IVa | Predominantly affecting limb, axial muscles, or both. May also have |
| | lesser involvement of oropharyngeal muscles. |
| IVb | Predominantly affecting oropharyngeal, respiratory muscles, or both. |
| | May also have lesser or equal involvement of limb, axial muscles, or |
| | both |
| Class V | Defined as intubation, with or without mechanical ventilation, except |
| | when employed during routine postoperative management. The use of a |
| | feeding tube without intubation places the patient in class IVb. |

Within the generalized categories II, III, and IV, patients are subclassified as class A if their symptoms are predominantly generalized or class B if their symptoms are predominantly bulbar (Jaretzki et al., Neurology, 2000, 55(1):16-23). The MGFA also has a system to classify patients based on postintervention outcomes and includes remission, defined as 1 year or longer without signs or symptoms and without any symptomatic (pyridostigmine) treatment, and which can be divided in complete (no pharmacologic treatment at all) or pharmacologic remission. Minimal manifestation status is defined as minimal signs or symptoms (no specific timeframe was defined) and pyridostigmine use may be accepted. Additionally, patients can be improved, unchanged, worse, experiencing an MG exacerbation, or have died of MG (Jaretzki et al., Neurology, 2000, 55(1):16-23). In some studies, MGFA class 0 has also been used to represent patients that have no symptoms (either due to stable treatment with e.g. Mestinon or due to remission) (Boldingh et al., Health and Quality of Life Outcomes, 2015, 13:115; Al-Moallem et al., Ann Saudi Med 2008, 28(5):341-345).

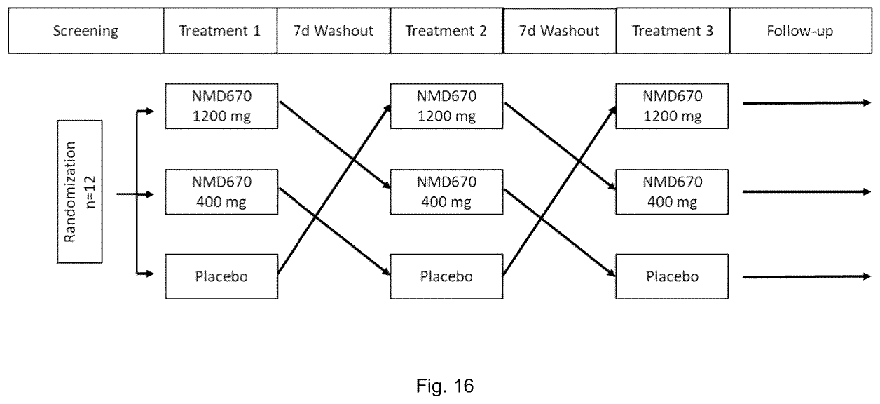

The clinical trial consisted of three study periods during which subjects received either a single dose of NMD670 or placebo. Single doses were administered in randomized order (400 mg free acid, 1200 mg free acid, or placebo p.o.), with a 7-day washout period between visits (FIG. 16). Assessment of the effect of NMD670 was determined using the Quantitative Myasthenia Gravis (QMG) score card as shown in Table 1.

The Quantitative Myasthenia Gravis (QMG) test is a standardized quantitative strength scoring system that was developed in the context of a clinical trial in MG and originally had 8 items (Besinger et al., Neurology 1983, 33(10):1316-21); it was later modified for a trial of cyclosporine (Tindall et al., N Eng/J Med 1987, 316(12):719-24), increasing the number of items to 13. This measure was modified again by Barohn and colleagues (Barohn et al., Ann N Y Acad Sci 1998, 841:769-72) making the 13 items all based on the examination, and this is the version currently in use (see table 1). The QMG test has several items that measure muscle strength, endurance or fatigability. The items measure the following symptoms and signs: ptosis, diplopia, double vision, swallowing, speech/onset of dysarthria, percent predicted forced vital capacity, grip strength (2 items), arm muscle endurance (2 items), leg muscle endurance (2 items), and head lifting endurance. All items are scored on a scale of 0 to 3, and total scores range from 0 to 39; higher scores indicate greater disease severity.

TABLE 1
Quantitative Myasthenia Gravis Score Card

| Test Items Weakness | | None | Mild | Moderate | Severe |
|---|---|---|---|---|---|
| Score | | 0 | 1 | 2 | 3 |
| Double vision | | >60 | 11-60 | 1-10 | Spontaneous |
| (lateral gaze) sec. | | | | | |
| Ptosis | | >60 | 11-60 | 1-10 | Spontaneous |
| (upward gaze) sec. | | | | | |
| Facial muscles | | Normal lid | Complete, | Complete, | Incomplete |
| | | closure | weak, | without | |
| | | | some | resistance | |
| | | | resistance | | |
| Swallowing | | Normal | Occasional | Consistent | Cannot |
| | | | choking | choking | swallow |
| Head, lifted | | >120 | >30-120 | >0-30 | 0 |
| (45° supine) sec. | | | | | |
| Right arm outstretched | | >240 | >90-240 | >10-90 | 0-10 |
| (90° standing) sec. | | | | | |
| Left arm outstretched | | >240 | >90-240 | >10-90 | 0-10 |
| (90° standing) sec. | | | | | |
| Speech following | | None | Dysarthria | Dysarthria | Dysarthria |
| counting aloud from | | at #50 | at #30-49 | at #10-29 | at #9 |
| 1 to 50 (onset of dysarthria) | | | | | |
| Right leg outstretched | | >100 | 31-100 | 1-30 | 0 |
| (45° supine) sec. | | | | | |
| Left leg outstretched | | >100 | 31-100 | 1-30 | 0 |
| (45° supine) sec. | | | | | |
| Vital capacity | male | >3.5 | >2.5-3.5 | 1.5-2.5 | <1.5 |
| | female | >2.5 | >1.8-2.5 | 1.2-1.8 | <1.2 |
| Right hand grip | male | >45 | >15-45 | 5-15 | <5 |
| (Kg force) | female | >31 | >10-30 | 5-10 | <5 |
| Left hand grip | male | >35 | >15-35 | 5-15 | <5 |
| (Kg force) | female | >25 | >10-25 | 5-10 | <5 |

From the discoveries provided by the above studies, the inventors were able to develop the compositions for use in MG treatment methods and MG treatment methods described herein. Exemplified embodiments of these compositions and methods are provided below. The compositions and methods described herein are not intended to be limited to the following exemplary embodiments.

### Compositions for Use

One aspect of the present disclosure relates to compositions for use in a method of treatment of myasthenia gravis in a subject. These compositions for use comprise administering a therapeutically effective dose of (2S)-2-[4-bromo-2-(1,2-oxazol-3-yl)phenoxy]propanoic acid (NMD670) to the patient, wherein the therapeutic dose is within the range of 100 mgs to 1500 mgs.

Thus one aspect of the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein the composition is for administration at a therapeutic dose of 100 to 1500 mg of (2S)-2-[4-bromo-2-(1,2-oxazol-3-yl)phenoxy]propanoic acid.

In exemplary embodiments, the therapeutically effective dose of NMD670 that is administered to the patient is less than 1500 mg, less than 1450 mg, less than 1300 mg, less than 1250 mg, less than 1200 mg, less than 1150 mg, less than 1100 mg, less than 1050 mg, less than 1000 mg, less than 950 mg, less than 900 mg, less than 850 mg, less than 800 mg, less than 750 mg, less than 700 mg, less than 650 mg, less than 600 mg, less than 550 mg, less than 500 mg, less than 450 mg, less than 400 mg, less than 350 mg, less than 300 mg, or less than 250 mg.

In other embodiments, the therapeutically effective dose of NMD670 that is administered to the patient is at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1000 mg, at least 1050 mg, at least 1100 mg, at least 1150 mg, at least 1200 mg, at least 1250 mg, at least 1300 mg, at least 1350 mg, at least 1400 mg, or at least 1450 mg.

In some exemplary embodiments, the therapeutically effective dose of NMD670 that is administered to the patient is from 100 to 600 mg, from 200 to 600 mg, from 250 to 550 mg, from 300 to 500 mg, from 350 to 450 mg, from 375 to 425 mg, or 400 mg. In other exemplary embodiments, the therapeutically effective dose of NMD670 that is administered to the patient is from 700 to 1400 mg, from 800 to 1350 mg, from 900 to 1300 mg, from 1000 to 1250 mg, from 1100 to 1250 mg, or about 1200 mg. In exemplary embodiments, the therapeutically effective dose of NMD670 that is administered to the patient is about 100 mg. In exemplary embodiments, the therapeutically effective dose of NMD670 that is administered to the patient is about 150 mg. In exemplary embodiments, the therapeutically effective dose of NMD670 that is administered to the patient is about 200 mg. In exemplary embodiments, the therapeutically effective dose of NMD670 that is administered to the patient is about 250 mg. In exemplary embodiments, the therapeutically effective dose of NMD670 that is administered to the patient is about 300 mg. In exemplary embodiments, the therapeutically effective dose of NMD670 that is administered to the patient is about 350 mg. In exemplary embodiments, the therapeutically effective dose of NMD670 that is administered to the patient is about 400 mg. In exemplary embodiments, the therapeutically effective dose of NMD670 that is administered to the patient is about 500 mg. In exemplary embodiments, the therapeutically effective dose of NMD670 that is administered to the patient is about 600 mg.

In exemplary embodiments, the therapeutic dose is to be administered at least one time daily. In exemplary embodiments, the therapeutic dose is to be administered one time daily. In exemplary embodiments, the therapeutic dose is to be administered two times daily. In exemplary embodiments, the therapeutic dose is to be administered three times daily. In exemplary embodiments, the therapeutic dose is to be administered four times daily.

In exemplary embodiments, the therapeutic dose is administered one time daily, i.e. the therapeutic dose is the total daily dosage. In exemplary embodiments, the therapeutic dose is 100 to 600 mg and is administered one time daily. In exemplary embodiments, the therapeutic dose is 200 to 600 mg and is administered one time daily. In exemplary embodiments, the therapeutic dose is 300 to 500 mg and is administered one time daily. In exemplary embodiments, the therapeutic dose is about 100 mg and is administered one time daily. In exemplary embodiments, the therapeutic dose is about 150 mg and is administered one time daily. In exemplary embodiments, the therapeutic dose is about 200 mg and is administered one time daily. In exemplary embodiments, the therapeutic dose is about 250 mg and is administered one time daily. In exemplary embodiments, the therapeutic dose is about 300 mg and is administered one time daily. In exemplary embodiments, the therapeutic dose is about 350 mg and is administered one time daily. In exemplary embodiments, the therapeutic dose is about 400 mg and is administered one time daily. In exemplary embodiments, the therapeutic dose is about 500 mg and is administered one time daily. In exemplary embodiments, the therapeutic dose is about 600 mg and is administered one time daily.

In exemplary embodiments, the therapeutic dose is administered two times daily, i.e. the total daily dosage is twice the therapeutic dose. In exemplary embodiments, the therapeutic dose is 100 to 600 mg and is administered two times daily. In exemplary embodiments, the therapeutic dose is 200 to 600 mg and is administered two times daily. In exemplary embodiments, the therapeutic dose is 300 to 500 mg and is administered two times daily. In exemplary embodiments, the therapeutic dose is about 100 mg and is administered two times daily. In exemplary embodiments, the therapeutic dose is about 150 mg and is administered two times daily. In exemplary embodiments, the therapeutic dose is about 200 mg and is administered two times daily. In exemplary embodiments, the therapeutic dose is about 250 mg and is administered two times daily. In exemplary embodiments, the therapeutic dose is about 300 mg and is administered two times daily. In exemplary embodiments, the therapeutic dose is about 350 mg and is administered two times daily. In exemplary embodiments, the therapeutic dose is about 400 mg and is administered two times daily. In exemplary embodiments, the therapeutic dose is about 500 mg and is administered two times daily. In exemplary embodiments, the therapeutic dose is about 600 mg and is administered two times daily.

In exemplary embodiments, the therapeutic dose is administered three times daily, i.e. the total daily dosage is three times the therapeutic dose. In exemplary embodiments, the therapeutic dose is 100 to 600 mg and is administered three times daily. In exemplary embodiments, the therapeutic dose is 200 to 600 mg and is administered three times daily. In exemplary embodiments, the therapeutic dose is 300 to 500 mg and is administered three times daily. In exemplary embodiments, the therapeutic dose is about 100 mg and is administered three times daily. In exemplary embodiments, the therapeutic dose is about 150 mg and is administered three times daily. In exemplary embodiments, the therapeutic dose is about 200 mg and is administered three times daily. In exemplary embodiments, the therapeutic dose is about 250 mg and is administered three times daily. In exemplary embodiments, the therapeutic dose is about 300 mg and is administered three times daily. In exemplary embodiments, the therapeutic dose is about 350 mg and is administered three times daily. In exemplary embodiments, the therapeutic dose is about 400 mg and is administered three times daily. In exemplary embodiments, the therapeutic dose is about 500 mg and is administered three times daily. In exemplary embodiments, the therapeutic dose is about 600 mg and is administered three times daily.

The composition comprising the therapeutic dose may be administered in one or more unit dosage forms. A therapeutic dose of 400 mg may for example be administered as one unit dosage form comprising 400 mg, or two unit dosage forms comprising 200 mg, or four unit dosage forms comprising 100 mg.

In exemplary embodiments, the composition for use is dosed orally. In exemplary embodiments, the composition for use is a solid dosage form. In exemplary embodiments, the solid dosage form is dosed orally. In exemplary embodiments, the solid dosage form is selected from the group consisting of capsule (such as sprinkle capsule and gelatine capsule), tablet (such as uncoated tablet, coated tablet, slow-release tablet) and sprinkle. In exemplary embodiments, the composition is in the form of a liquid, liquid suspension, oil, emulsion, or syrup. In exemplary embodiments, the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 5 hours, such 1.5 to 4 hours, such as about 2 hours or about 3 hours, after administration.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 13,000 to 32,000 ng/mL, such as 15,000 to 30,000 ng/mL, such as 15,730 to 27,670 ng/mL, such as 16,000 to 27,000 ng/mL, such as 17,360 to 27,125 ng/mL, such as 18,000 to 25,000 ng/mL, such as 20,000 to 23,000 ng/mL, such as about 21,700 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean Cₘₐₓ is 21,700 ng/mL and the standard deviation is 5,970 ng/mL.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 21,700 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 40,000 to 110,000 ng/mL, such as 46,300 to 107,100 ng/mL, such as 50,000 to 100,000 ng/mL, such as 60,000 to 90,000 ng/mL, such as 61,360 to 95,875 ng/mL, such as 70,000 to 80,000 ng/mL, such as about 76,700 ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean Cₘₐₓ is 77,700 ng/mL and the standard deviation is 30,400 ng/mL. In exemplary embodiments, mean Cₘₐₓ is 76,700 ng/mL and the standard deviation is 30,400 ng/mL.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 76,700 ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 60,000 to 130,000 h·ng/mL, such as 64,100 to 123,300 h·ng/mL, such as 70,000 to 120,000 h·ng/mL, such as 74,960 to 117,125 h·ng/mL, such as 80,000 to 110,000 h·ng/mL, such as 90,000 to 100,000 h·ng/mL, such as about 93,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean AUC_{0-infinity} is 93,700 ng/mL and the standard deviation is 29,600 ng/mL.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 93,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 250,000 to 500,000 h·ng/mL, such as 265,000 to 491,000 h·ng/mL, such as 280,000 to 480,000 h·ng/mL, such as 302,400 to 472,500 h·ng/mL, such as 320,000 to 450,000 h·ng/mL, such as 340,000 to 425,000 h·ng/mL, such as 360,000 to 400,000 h·ng/mL, such as about 378,300 h·ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, mean AUC_{0-infinity} is 378,000 ng/mL and the standard deviation is 113,000 ng/mL.

In exemplary embodiments, the composition for use is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 378,300 h·ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition or use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 6 hours after administration, such as after about 2 hours after administration.

In exemplary embodiments, the composition or use is to be administered orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 3 to 7 hours after administration.

In exemplary embodiments, the AUC_{0-24 infinity}, Cₘₐₓ or Tₘₐₓ is measured after administration of a single dose to a human subject suffering from myasthenia gravis.

The compositions for use described herein can be formulated for administrating either orally, parenterally, intravenously, inhaled, topically, enterally, rectally, buccally or as an aerosol.

In exemplary embodiments, the composition for use further comprises at least one pharmaceutically acceptable adjuvant and/or excipient. In exemplary embodiments, the composition for use comprises at least one pharmaceutically acceptable adjuvant and/or excipient selected from the group consisting of filler, binder, lubricant and disintegrant. In exemplary embodiments, the composition for use comprises at least one pharmaceutically acceptable adjuvant and/or excipient selected from the group consisting of silicified microcrystalline cellulose, microcrystalline cellulose, maltodextrin, magnesium stearate and croscarmellose sodium.

In exemplary embodiments, the composition for use comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use comprises 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant; and
e. 0.25 to 5 wt %, such as 0.3 to 2.5 wt % disintegrant;
   with the proviso that the sum of the wt % of the components does not exceed 100 wt %.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant;
e. 0.25 to 5 wt %, such as 0.3 to 2.5 wt % disintegrant; and
f. 1 to 10 wt % film coating;
   with the proviso that the sum of the wt % of the components does not exceed 100 wt %.

In exemplary embodiments, the composition for use is in the form of one or more solid dosage forms comprising:
a. 10 to 80 wt %, such as 40 to 65 wt %, such as 50 to 60 wt %, such as 50 to 55 wt %, such as 55 to 60 wt % such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
   with the proviso that the sum of the wt % of the components does not exceed 100 wt %.

In exemplary embodiments, the composition for use is in the form of a solid dosage form and comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
   with the proviso that the sum of the wt % of the components does not exceed 100 wt %.

In exemplary embodiments, the composition for use is in the form of one or more solid dosage forms comprising or consisting of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
g. 1 to 10 wt % film coating composition such as Opadry white,
   with the proviso that the sum of the wt % of the components does not exceed 100 wt %.

In exemplary embodiments, the composition for use is in the form of a solid dosage form and comprises or consists of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate;
f. 0.25 to 5 wt %, such as 0.3 to 2.5 wt % Croscarmellose sodium; and
g. 1 to 10 wt % film coating composition such as Opadry white,
   with the proviso that the sum of the wt % of the components does not exceed 100 wt %.

In exemplary embodiments, the subject has a level of serum uric acid below 6.5 mg/dL.

In exemplary embodiments, the Myasthenia Gravis Foundation of America (MGFA) clinical classification of the subject is class I, IIa, IIb IIIa, IIIb, IVa, IVb or V prior to treatment. In exemplary embodiments, the Myasthenia Gravis Foundation of America (MGFA) clinical classification of the subject is class I, IIa, IIb IIIa, IIIb, IVa, IVb or V prior to treatment. In exemplary embodiments, the Myasthenia Gravis Foundation of America (MGFA) clinical classification of the subject is class I, IIa, IIb, or IIIb prior to treatment.

In exemplary embodiments, the subject experiences a reduction in Quantitative Myasthenia Gravis total score after treatment. In exemplary embodiments, the subject experiences a reduction in Quantitative Myasthenia Gravis total score after treatment, wherein the reduction in QMG total score after treatment is at least 0.9 points, such as at least 1.0 point, such as at least 1.5 points, such as at least 2.0 points, such as at least 3.0 points, between 2 and 5 hours, such as 2 hours after treatment, such as 3 hours after treatment, such as 4 hours after treatment or such as 5 hours after treatment.

In exemplary embodiments, the Quantitative Myasthenia Gravis (QMG) total score is reduced compared to placebo at the same time point after treatment. In exemplary embodiments, the Quantitative Myasthenia Gravis (QMG) total score is reduced compared to placebo at the same time point after treatment, and reduction in QMG total score after treatment is at least 0.9 points, such as at least 1.0 point, such as at least 1.5 points, such as at least 2.0 points, such as at least 3.0 points, between 2 and 5 hours, such as 2 hours after treatment, such as 3 hours after treatment, such as 4 hours after treatment or such as 5 hours after treatment.

In exemplary embodiments, the subject experiences a decrease in QMG total score after treatment with NMD670. In exemplary embodiments, a decrease in the QMG total score can be determined by comparing the change from baseline in the QMG total score after a defined period of time (e.g., 21 days) of NMD670 treatment with the change from baseline in the QMG total score after a defined period of time (e.g., 21 days) of placebo treatment. In exemplary embodiments, the subject experiences a decrease in the QMG total score after treatment with NMD670, wherein the score has decreased by at least at least 0.9 points, such as at least 1.0 point, such as at least 1.5 points, such as at least 2.0 points, such as at least 3.0 points. In exemplary embodiments, the subject experiences a decrease in QMG total score after between 2 and 5 hours, such as 2 hours after treatment, such as 3 hours after treatment, such as 4 hours after treatment or such as 5 hours after treatment with NMD670.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Quantitative Myasthenia Gravis (QMG) total score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Quantitative Myasthenia Gravis (QMG) total score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered one time daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered one time daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered two times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered two times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered three times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered three times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid; and
the composition is to be administered four times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid; and
the composition is to be administered four times daily.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered one time daily; and the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered one time daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered three times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered three times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered four times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered four times daily; and
the composition is in the form of a solid dosage form and is to be dosed orally.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; the subject experiences a reduction in Quantitative Myasthenia Gravis total score after treatment, wherein the reduction in QMG total score after treatment is at least 0.9 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally;
the subject experiences a reduction in Quantitative Myasthenia Gravis total score after treatment, wherein the reduction in QMG total score after treatment is at least 0.9 points.

In exemplary embodiments, the subject experiences a decrease in the MG activities of daily living profile (MG-ADL) score after treatment with NMD670 (Wolfe et al., Neurology 1999, 52(7):1487-9; Muppidi et al., Muscle Nerve, 2022, 65(6):630-639). In exemplary embodiments, a decrease in the MG-ADL score can be determined by comparing the change from baseline in the MG-ADL score after a defined period of time (e.g., 21 days) of NMD670 treatment with the change from baseline in the MG-ADL score after a defined period of time (e.g., 21 days) of placebo treatment. In exemplary embodiments, the subject experiences a decrease in the MG-ADL score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the MG activities of daily living profile score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the MG activities of daily living profile score.

In exemplary embodiments, the subject or group of subjects experience an increase in muscle strength after treatment with NMD670. Grip strength is a measure of muscular strength, or the maximum force/tension generated by one's forearm muscles. It can be used as a screening tool for the measurement of upper body strength and overall strength. In exemplary embodiments, increase in muscle strength can also be determined by measuring the strength of the thigh (knee flexors), the upper arm (elbow flexor and extension) and/or the shoulder (shoulder abduction). In exemplary embodiments, the subject or group of subjects experience an increase in muscle strength after treatment with NMD670 when determined by measuring grip strength using a handheld dynamometer (see Example 13 and Besinger et al., Neurology 1983, 33(10):1316-21; Tindall et al., N Engl J Med 1987, 316(12):719-24 and Barohn et al., Ann N Y Acad Sci 1998, 841:769-72). In exemplary embodiments, an increase in muscle strength can be determined by comparing the change from baseline in muscle strength after a defined period of time (e.g., 21 days) of NMD670 treatment with the change from baseline in muscle strength after a defined period of time (e.g., 21 days) of placebo treatment. In exemplary embodiments the subject or group of subjects experience an increase in muscle strength after treatment with NMD670 when determined by measuring grip strength using a handheld dynamometer, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.

In exemplary embodiments the subject or group of subjects experience an increase in muscle strength after treatment with NMD670 when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments the subject or group of subjects experience an increase in muscle strength after treatment with NMD670 when determined by measuring knee flexor strength using a handheld dynamometer, wherein knee flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as at least 5.0 kg, such as at least 7.5 kg, such as between 0.25 and 15.0 kg, such as between 0.25 and 10.0 kg, such as between 0.5 and 15.0 kg.

In exemplary embodiments the subject or group of subjects experience an increase in muscle strength after treatment with NMD670 when determined by measuring elbow flexor strength using a handheld dynamometer, wherein elbow flexor strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and the subject experiences an increase in muscle strength.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and the subject experiences an increase in muscle strength.

In exemplary embodiments, the subject experiences a decrease in the Myasthenia Gravis Composite (MGC) scale after treatment with NMD670 (Burns et al, Neurology 2010, 74(18):1434-40). In exemplary embodiments, a decrease in the MGC scale can be determined by comparing the change from baseline in the MGC scale after a defined period of time (e.g., 21 days) of NMD670 treatment with the change from baseline in the MGC scale after a defined period of time (e.g., 21 days) of placebo treatment. In exemplary embodiments, the subject experiences a decrease in the MGC scale after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Myasthenia Gravis Composite scale.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Myasthenia Gravis Composite scale.

In exemplary embodiments, the subject experiences a decrease in the Myasthenia Gravis Quality of Life 15 (MG-QOL15) score after treatment with NMD670 (Burns et al, Muscle Nerve, 2008, 38(2):957-63; Diez Porras et al, J Clin Med. 2022, 11(8):2189). In exemplary embodiments, a decrease in the MG-QOL15 score can be determined by comparing the change from baseline in the MG-QOL15 score after a defined period of time (e.g., 21 days) of NMD670 treatment with the change from baseline in the MG-QOL15 score after a defined period of time (e.g., 21 days) of placebo treatment. In exemplary embodiments, the subject experiences a decrease in the MG-QOL15 score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and the subject experiences a decrease in the Myasthenia Gravis Quality of Life 15 score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and the subject experiences a decrease in the Myasthenia Gravis Quality of Life 15 score.

In exemplary embodiments, the subject experiences an increase in health state when determined using the EQ-5D scale after treatment with NMD670 (Rabin and Charro, Ann Med. 2001, 33(5):337-43). In exemplary embodiments, an increase in the EQ-5D scale can be determined by comparing the change from baseline in the EQ-5D scale after a defined period of time (e.g., 21 days) of NMD670 treatment with the change from baseline in the EQ-5D scale after a defined period of time (e.g., 21 days) of placebo treatment. In exemplary embodiments, the subject experiences an increase in the EQ-5D scale after treatment with NMD670, wherein the score has increased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences increase in health state when determined using the EQ-5D scale.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences increase in health state when determined using the EQ-5D scale.

In exemplary embodiments, the subject experiences a reduction in jitter after treatment with NMD670. In exemplary embodiments, the subject experiences a reduction in jitter after treatment with NMD670 when determined using single fibre electromyography (Sanders et al, Clin Neurophysiol. 2019, 130(8):1417-1439). In exemplary embodiments, a reduction in jitter can be determined by comparing the change from baseline in jitter after a defined period of time (e.g., 21 days) of NMD670 treatment with the change from baseline in jitter after a defined period of time (e.g., 21 days) of placebo treatment. In exemplary embodiments the subject experiences a reduction in jitter after treatment with NMD670 when determined using single fibre electromyography, wherein jitter has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments the subject experiences a reduction in jitter after treatment with NMD670 when determined using single fibre electromyography, wherein jitter has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in jitter when determined using single fibre electromyography.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in jitter when determined using single fibre electromyography.

In exemplary embodiments, the subject experiences a reduction in blocking after treatment with NMD670. In exemplary embodiments, the subject experiences a reduction in blocking after treatment with NMD670 when determined using single fibre electromyography (Sanders et al, Clin Neurophysiol. 2019, 130(8):1417-1439). In exemplary embodiments, a reduction in blocking can be determined by comparing the change from baseline in blocking after a defined period of time (e.g., 21 days) of NMD670 treatment with the change from baseline in blocking after a defined period of time (e.g., 21 days) of placebo treatment. In exemplary embodiments the subject experiences a reduction in blocking after treatment with NMD670 when determined using single fibre electromyography, wherein blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in blocking when determined using single fibre electromyography.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a reduction in blocking when determined using single fibre electromyography.

In exemplary embodiments, the subject a decrease in the Individualised Neuromuscular Quality of Life score after treatment with NMD670 (Vincent et al, Neurology, 2007, 68(13):1051-7). In exemplary embodiments, a decrease in the Individualised Neuromuscular Quality of Life score can be determined by comparing the change from baseline in the Individualised Neuromuscular Quality of Life score after a defined period of time (e.g., 21 days) of NMD670 treatment with the change from baseline the Individualised Neuromuscular Quality of Life score after a defined period of time (e.g., 21 days) of placebo treatment. In exemplary embodiments, the subject experiences a decrease in the Individualised Neuromuscular Quality of Life score after treatment with NMD670, wherein the score has decreased 0.5 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Individualised Neuromuscular Quality of Life score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be dosed orally; and
the subject experiences a decrease in the Individualised Neuromuscular Quality of Life score.

In exemplary embodiments, the subject or group of subjects experience a decrease in the Fatigue Severity Scale score after treatment with NMD670 (Werlauff et al, Qual Life Res., 2014, 23:1479-1488). In exemplary embodiments, a decrease in the Fatigue Severity Scale score can be determined by comparing the change from baseline in the Fatigue Severity Scale score after a defined period of time (e.g., 21 days) of NMD670 treatment with the change from baseline the Fatigue Severity Scale score after a defined period of time (e.g., 21 days) of placebo treatment. In exemplary embodiments, the subject or group of subjects experience a decrease in the Fatigue Severity Scale score after treatment with NMD670, wherein the score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a decrease in the Fatigue Severity Scale score.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences a decrease in the Fatigue Severity Scale score.

In exemplary embodiments, the subject or group of subjects experience an improvement in pulmonary function after treatment with NMD670 (Oliveira et al, Neuromuscul Disord. 2017, 27(2):120-127; Neder et al, Braz J Med Biol Res. 1999, 32(6):719-27). In exemplary embodiments, an improvement in pulmonary function can be determined by comparing the change from baseline of pulmonary function after a defined period of time (e.g., 21 days) of NMD670 treatment with the change from baseline of pulmonary function after a defined period of time (e.g., 21 days) of placebo treatment. In exemplary embodiments, the subject or group of subjects experience an improvement in pulmonary function after treatment with NMD670 when determined by measuring forced vital capacity (FVC), wherein FVC has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments, the subject or group of subjects experience an improvement in pulmonary function after treatment with NMD670 when determined by measuring forced expiratory volume in 1 second (FEV1), wherein FEV1 has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments, the subject or group of subjects experience an improvement in pulmonary function after treatment with NMD670 when determined by measuring maximal inspiratory pressure (MIP), wherein MIP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments, the subject or group of subjects experience an improvement in pulmonary function after treatment with NMD670 when determined by measuring maximal expiratory pressure (MEP), wherein MEP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 100 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an improvement in pulmonary function.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein
the composition is for administration at a therapeutic dose of 200 to 600 mg of (2S)-2-[4-bromo-2-(1,2-oxazol-3-yl)phenoxy]propanoic acid;
the composition is to be administered two times daily;
the composition is in the form of a solid dosage form and is to be administered orally; and
the subject experiences an improvement in pulmonary function.

In exemplary embodiments, the present disclosure relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein the subject has a level of serum uric acid below 6.5 mg/dL. In exemplary embodiments, the composition for use is for administration at a therapeutic dose of 100 to 1500 mg of (2S)-2-[4-bromo-2-(1,2-oxazol-3-yl)phenoxy]propanoic acid.

In exemplary embodiments, the composition for use is for administration at a therapeutic dose as defined herein.

In one aspect, the present invention relates to use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of myasthenia gravis in a subject, wherein the composition is for administration at a therapeutic dose of 100 to 1500 mg of (2S)-2-[4-bromo-2-(1,2-oxazol-3-yl)phenoxy]propanoic acid.

### Pharmaceutical Compositions

Another aspect of the present disclosure relates to compositions comprising a therapeutically effective dose of NMD670 for use in treating or ameliorating symptoms of myasthenia gravis in a patient suffering from myasthenia gravis. A further aspect of the present disclosure relates to compositions comprising a therapeutically effective dose of NMD670 in treating or ameliorating symptoms of myasthenia gravis in a patient suffering from myasthenia gravis. All of the following exemplified embodiments of the composition can be used in the treatment methods described herein.

In one aspect, the present invention relates to a composition comprising (2S)-2-[4-bromo-2-(1,2-oxazol-3-yl)phenoxy]propanoic acid, or a pharmaceutically acceptable salt, hydrate, polymorph, tautomer, or solvate thereof. In one embodiment, the composition comprises 50 to 400 mg (2S)-2-[4-bromo-2-(1,2-oxazol-3-yl)phenoxy]propanoic acid, or a pharmaceutically acceptable salt, hydrate, polymorph, tautomer, or solvate thereof. In one aspect, the present invention relates to a composition, formulated as a solid dosage form, comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition comprises 50 to 400 mg (2S)-2-[4-bromo-2-(1,2-oxazol-3-yl)phenoxy]propanoic acid. In exemplary embodiments, the composition further comprises at least one pharmaceutically acceptable adjuvant and/or excipient. In exemplary embodiments, the composition is for oral administration. In exemplary embodiments, the composition further comprises a pharmaceutically acceptable adjuvant and/or excipient selected from the group consisting of filler, binder, lubricant and disintegrant. In exemplary embodiments, the composition comprises a pharmaceutically acceptable adjuvant and/or excipient selected from the group consisting of silicified microcrystalline cellulose, microcrystalline cellulose, maltodextrin, magnesium stearate and croscarmellose sodium.

In exemplary embodiments, the composition comprises 50 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 100 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 150 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 200 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 250 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 300 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 350 mg (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 400 mg (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition comprises 10% to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof. In exemplary embodiments, the composition comprises 10% to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant; and
e. 0.25 to 5 wt %, such as 0.3 to 2.5 wt % disintegrant;
   with the proviso that the sum of the wt % of the components does not exceed 100 wt %.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 20 to 80 wt %, such as 25 to 50 wt % filler;
c. 2 to 20 wt %, such as 3 to 16 wt % binder;
d. 0.25 to 3 wt %, such as 0.4 to 2.0 wt % lubricant;
e. 0.25 to 5 wt %, such as 0.3 to 2.5 wt % disintegrant; and
f. 1 to 10 wt % film coating;
   with the proviso that the sum of the wt % of the components does not exceed 100 wt %.

In exemplary embodiments, the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
   with the proviso that the sum of the wt % of the components does not exceed 100 wt %.

In exemplary embodiments, the composition comprises or consists of:
a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate;
f. 0.25 to 5 wt %, such as 0.3 to 2.5 wt % Croscarmellose sodium; and
g. 1 to 10 wt % film coating composition such as Opadry white,
   with the proviso that the sum of the wt % of the components does not exceed 100 wt %.

In exemplary embodiments, the present disclosure relates to a composition comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mgs to 1500 mgs.

In exemplary embodiments, the present disclosure relates to a composition, formulated as a solid dosage form, comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 50 mg to 400 mg. In exemplary embodiments, the therapeutically effective dose is 100 mgs. In exemplary embodiments, the therapeutically effective dose is 150 mgs. In exemplary embodiments, the therapeutically effective dose is 200 mgs. In exemplary embodiments, the therapeutically effective dose is 250 mgs. In exemplary embodiments, the therapeutically effective dose is 300 mgs. In exemplary embodiments, the therapeutically effective dose is 350 mgs. In exemplary embodiments, the therapeutically effective dose is 400 mgs. In exemplary embodiments, the therapeutically effective dose is given once daily. In exemplary embodiments, the therapeutically effective dose is given twice daily. In exemplary embodiments, the therapeutically effective dose is given three times daily.

In exemplary embodiments, the present disclosure relates to a composition, formulated as a solid dosage form, comprising 50 mg to 400 mg of (2S)-2-[4-bromo-2-(1,2-oxazol-3-yl)phenoxy]propanoic acid or a pharmaceutically acceptable salt, hydrate, polymorph, tautomer, or solvate thereof. In exemplary embodiments, the solid dosage form comprises 100 mg of NMD670. In exemplary embodiments, the solid dosage form comprises 150 mg of NMD670. In exemplary embodiments, the solid dosage form comprises 200 mg of NMD670. In exemplary embodiments, the solid dosage form comprises 250 mg of NMD670. In exemplary embodiments, the solid dosage form comprises 300 mg of NMD670. In exemplary embodiments, the solid dosage form comprises 350 mg of NMD670. In exemplary embodiments, the solid dosage form comprises 400 mg of NMD670.

In exemplary embodiment, the composition is a solid dosage form. In exemplary embodiments, the solid dosage form is selected from the group consisting of capsule (such as sprinkle capsule and gelatine capsule), tablet (such as uncoated tablet, coated tablet, slow-release tablet) and sprinkle. In exemplary embodiments, the composition is in the form of a liquid, liquid suspension, oil, emulsion, or syrup. In exemplary embodiments, the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.

### Methods

In exemplary embodiments, the methods for treating a patient suffering from symptoms of myasthenia gravis may result in an improvement of the patient's quantitative myasthenia gravis total score, hand grip strength, compound muscle action potentials and/or muscle decrement. In exemplary embodiments, the methods for treating a patient suffering from symptoms of myasthenia gravis may result in a reduction in Quantitative Myasthenia Gravis total score; a decrease in the MG activities of daily living profile (MG-ADL) score; an increase in muscle strength; a decrease in the Myasthenia Gravis Composite (MGC) scale; experiences a decrease in the Myasthenia Gravis Quality of Life 15 (MG-QOL15) score; an increase in health state when determined using the EQ-5D scale; a reduction in jitter; a reduction in blocking; a decrease in the Individualised Neuromuscular Quality of Life score; a decrease in the Fatigue Severity Scale score and/or an improvement in pulmonary function.

Accordingly, an aspect of the present disclosure relates to methods for treating myasthenia gravis that result in an improvement of a quantitative myasthenia gravis total score in the patient, these methods comprising administering a therapeutically effective dose of NMD670 to the patient, wherein the therapeutic dose is within the range of 100 mgs to 1500 mgs.

In one aspect, the present disclosure relates to a method for treatment of myasthenia gravis in a subject in need thereof, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.

In one aspect, the present disclosure relates to a method for improving the Quantitative Myasthenia Gravis total score in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject. In exemplary embodiments, the reduction in QMG total score after treatment is at least 0.9 points, such as at least 1.0 point, such as at least 1.5 points, such as at least 2.0 points, such as at least 3.0 points. In exemplary embodiments, the reduction in QMG total score after treatment is at least 0.9 points, such as at least 1.0 point, such as at least 1.5 points, such as at least 2.0 points, such as at least 3.0 points compared to placebo at the same time point. In exemplary embodiments, the reduction in QMG total score after treatment is between 2 and 5 hours, such as 2 hours after treatment, such as 3 hours after treatment, such as 4 hours after treatment or such as 5 hours after treatment.

Another aspect of the present disclosure relates to methods for treating myasthenia gravis that result in an improvement of hand grip strength in a patient, these methods comprising administering a therapeutically effective dose of NMD670 to the patient, wherein the therapeutic dose is within the range of 100 mgs to 1500 mgs. In exemplary embodiments, the patient's hand grip strength improves by at least 10%, by at least 20%, by at least 30% or by at least 40% when compared to the patient's hand grip strength before administration of the therapeutically effective dose of NMD670. Measurement of hand grip is a part of the QMG test (see Example 13 and Besinger et al., Neurology 1983, 33(10):1316-21; Tindall et al., N Engl J Med 1987, 316(12):719-24 and Barohn et al., Ann N Y Acad Sci 1998, 841:769-72).

In one aspect, the present disclosure relates to a method of improving right hand grip strength in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject. In exemplary embodiments, the improvement in right hand grip strength after treatment is at least 2.0 kg, such as at least 2.5 kg. In exemplary embodiments, the improvement in right hand grip strength is at least 2.0 kg, such as at least 2.5 kg compared to placebo at the same time point.

Another aspect of the present disclosure relates to methods for treating myasthenia gravis that result in an improvement of compound muscle action potentials in the patient, these methods comprising administering a therapeutically effective dose of NMD670 to the patient, wherein the therapeutic dose is within the range of 100 mgs to 1500 mgs. In exemplary embodiments, the patient's compound muscle action potentials improve by at least 10%, by at least 20%, by at least 30% or by at least 40% when compared to the patient's compound muscle action potentials before administration of the therapeutically effective dose of NMD670. An improvement in compound muscle action potential can be determined via repetitive nerve simulation (see Example 13 and Niks et al., Muscle & Nerve, 2003, 28(2):236-238; Ruys-Van Oeyen et al., Muscle & Nerve, 2002, 26(2):279-282; Schumm et al., Muscle & Nerve, 1984, 7(2):147-151).

Another aspect of the present disclosure relates to methods for treating myasthenia gravis that result in recovery of muscle decrement in the patient, these methods comprising administering a therapeutically effective dose of NMD670 to the patient, wherein the therapeutic dose is within the range of 100 mgs to 1500 mgs. In exemplary embodiments, the patient's muscle decrement improves by at least 10%, by at least 20%, by at least 30% or by at least 40% when compared to the patient's muscle decrement before administration of the therapeutically effective dose of NMD670.

In one aspect, the present disclosure relates to a method of reducing decrement in EMG in repetitive nerve stimulation in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject. A recovery of muscle decrement can be determined via repetitive nerve stimulation (see Example 13 and Niks et al., Muscle & Nerve, 2003, 28(2):236-238; Ruys-Van Oeyen et al., Muscle & Nerve, 2002, 26(2):279-282; Schumm et al., Muscle & Nerve, 1984, 7(2):147-151).

In one aspect, the present disclosure relates to a method of improving the symptoms of double vision in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject. Measurement of double vision is a part of the QMG test (see Example 13 and Besinger et al., Neurology 1983, 33(10):1316-21; Tindall et al., N Eng/J Med 1987, 316(12):719-24 and Barohn et al., Ann N Y Acad Sci 1998, 841:769-72).

In one aspect, the present disclosure relates to a method of improving the symptoms of ptosis in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject. Measurement of ptosis is a part of the QMG test (see Example 13 and Besinger et al., Neurology 1983, 33(10):1316-21; Tindall et al., N Eng/J Med 1987, 316(12):719-24 and Barohn et al., Ann N Y Acad Sci 1998, 841:769-72).

In one aspect, the present disclosure relates to a method of improving the symptoms of dysarthria in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject. Measurement of dysarthria is a part of the QMG test (see Example 13 and Besinger et al., Neurology 1983, 33(10):1316-21; Tindall et al., N Eng/J Med 1987, 316(12):719-24 and Barohn et al., Ann N Y Acad Sci 1998, 841:769-72).

One aspect of the present disclosure relates to methods for treating myasthenia gravis that result in a decrease in the MG activities of daily living profile (MG-ADL) score, these methods comprising administering a therapeutically effective dose of NMD670 to the patient, wherein the therapeutic dose is within the range of 100 mg to 1500 mg. An improvement in the MG activities of daily living profile (MG-ADL) score is a decrease in score (Wolfe et al., Neurology 1999, 52(7):1487-9; Muppidi et al., Muscle Nerve, 2022, 65(6):630-639).

One aspect of the present disclosure relates to methods for treating myasthenia gravis that result in an increase in muscle strength, these methods comprising administering a therapeutically effective dose of NMD670 to the patient, wherein the therapeutic dose is within the range of 100 mg to 1500 mg. An improvement in muscle strength can be determined by measuring grip strength using a handheld dynamometer (see Example 13 and Besinger et al., Neurology 1983, 33(10):1316-21; Tindall et al., N Eng/J Med 1987, 316(12):719-24 and Barohn et al., Ann N Y Acad Sci 1998, 841:769-72).

One aspect of the present disclosure relates to methods for treating myasthenia gravis that result in a decrease in the Myasthenia Gravis Composite (MGC) scale, these methods comprising administering a therapeutically effective dose of NMD670 to the patient, wherein the therapeutic dose is within the range of 100 mg to 1500 mg. An improvement in the Myasthenia Gravis Composite (MGC) scale is a decrease in score (Burns et al, Neurology 2010, 74(18):1434-40).

One aspect of the present disclosure relates to methods for treating myasthenia gravis that result in a decrease in the Myasthenia Gravis Quality of Life 15 (MG-QOL15) score, these methods comprising administering a therapeutically effective dose of NMD670 to the patient, wherein the therapeutic dose is within the range of 100 mg to 1500 mg. An improvement in the Myasthenia Gravis Quality of Life 15 (MG-QOL15) score is a decrease in score (Burns et al, Muscle Nerve, 2008, 38(2):957-63; Diez Porras et al, J Clin Med. 2022, 11(8):2189).

One aspect of the present disclosure relates to methods for treating myasthenia gravis that result in an improvement in health state when determined using the EQ-5D scale, these methods comprising administering a therapeutically effective dose of NMD670 to the patient, wherein the therapeutic dose is within the range of 100 mg to 1500 mg. An improvement in health state can be determined using the EQ-5D scale and is an increase in score (Rabin and Charro, Ann Med. 2001, 33(5):337-43).

One aspect of the present disclosure relates to methods for treating myasthenia gravis that result in a reduction in jitter, these methods comprising administering a therapeutically effective dose of NMD670 to the patient, wherein the therapeutic dose is within the range of 100 mg to 1500 mg. A reduction in jitter can be determined using single fibre electromyography (Sanders et al, Clin Neurophysiol. 2019, 130(8):1417-1439).

One aspect of the present disclosure relates to methods for treating myasthenia gravis that result in a reduction in blocking, these methods comprising administering a therapeutically effective dose of NMD670 to the patient, wherein the therapeutic dose is within the range of 100 mg to 1500 mg. A reduction in blocking can be determined using single fibre electromyography (Sanders et al, Clin Neurophysiol. 2019, 130(8):1417-1439).

One aspect of the present disclosure relates to methods for treating myasthenia gravis that result in a decrease in the Individualised Neuromuscular Quality of Life score, these methods comprising administering a therapeutically effective dose of NMD670 to the patient, wherein the therapeutic dose is within the range of 100 mg to 1500 mg. An improvement in the Individualised Neuromuscular Quality of Life score is a decrease in score (Vincent et al, Neurology, 2007, 68(13):1051-7).

One aspect of the present disclosure relates to methods for treating myasthenia gravis that result in a decrease in the Fatigue Severity Scale score, these methods comprising administering a therapeutically effective dose of NMD670 to the patient, wherein the therapeutic dose is within the range of 100 mg to 1500 mg. An improvement in the Fatigue Severity Scale score is a decrease in score (Werlauff et al, Qual Life Res., 2014, 23:1479-1488).

One aspect of the present disclosure relates to methods for treating myasthenia gravis that result in an improvement in pulmonary function, these methods comprising administering a therapeutically effective dose of NMD670 to the patient, wherein the therapeutic dose is within the range of 100 mg to 1500 mg. An improvement in pulmonary function can be determined by measuring forced vital capacity (FVC), forced expiratory volume in 1 second (FEV1), maximal inspiratory pressure (MIP), and/or measuring maximal expiratory pressure (MEP) (Oliveira et al, Neuromuscul Disord. 2017, 27(2):120-127; Neder et al, Braz J Med Biol Res. 1999, 32(6):719-27).

In one aspect, the present disclosure relates to a method for enhancing neuromuscular transmission and/or restoration of skeletal muscle function, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.

In exemplary embodiments, the composition is administered at a therapeutic dose of 100 to 1500 mg of (2S)-2-[4-bromo-2-(1,2-oxazol-3-yl)phenoxy]propanoic acid.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered three times daily.

In exemplary embodiments, the composition is administered at a therapeutic dose of 100 to 600 mg of (2S)-2-[4-bromo-2-(1,2-oxazol-3-yl)phenoxy]propanoic acid and the composition is to be administered four times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered three times daily.

In exemplary embodiments, the composition is administered at a therapeutic dose of 200 to 600 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 100 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 150 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 200 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 200 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 250 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 250 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 300 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 300 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 350 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 350 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 400 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 400 mg of (2S)-2-[4-bromo-2-(1,2-oxazol-3-yl)phenoxy]propanoic acid and the composition is to be administered four times daily.

In exemplary embodiments, the composition is administered at a therapeutic dose of about 500 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 500 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 500 mg of (2S)-2-[4-bromo-2-(1,2-oxazol-3-yl)phenoxy]propanoic acid and the composition is to be administered three times daily.

In exemplary embodiments, the composition is administered at a therapeutic dose of about 600 mg of (2S)-2-[4-bromo-2-(1,2-oxazol-3-yl)phenoxy]propanoic acid and the composition is to be administered one time daily.

In exemplary embodiments, the composition is administered at a therapeutic dose of about 600 mg of (2S)-2-[4-bromo-2-(1,2-oxazol-3-yl)phenoxy]propanoic acid and the composition is to be administered two times daily.

In exemplary embodiments, the composition is administered at a therapeutic dose of about 600 mg of (2S)-2-[4-bromo-2-(1,2-oxazol-3-yl)phenoxy]propanoic acid and the composition is to be administered three times daily.

A recovery in a patient's muscle decrement can include, but is not limited to, an enhancement in their neuromuscular transmission and/or a restoration in their skeletal muscle function.

The methods of treatment disclosed herein may further comprise the administration of other active agents that are known to treat, prevent and/or ameliorate neuromuscular disorders. These active agents may be acetylcholine esterase inhibitors, such as neostigmine or pyridostigmine, immunosuppressive drugs, compounds used in anti-myotonic treatment, compounds for increasing the Ca²⁺ sensitivity of the contractile filaments in muscle, and/or compounds for increasing Ach release by blocking voltage gated K⁺ channels in the pre-synaptic terminal.

In one aspect, the present disclosure relates to a method for treatment of myasthenia gravis comprising administering (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor to a subject in need thereof.

In some exemplary embodiments, the methods of treatment comprise administering the therapeutically effective dose of NMD670 with one or more of the following compounds: amifampridine, Prednisolone, Prednisone, Azathioprine, Soliris, Rituximab, Efgartigimod alfa, Zilucoplan, Rozanolixizumab, Cholecalciferol and immunoglobulin.

In exemplary embodiments, the methods of treatment comprise administering the therapeutically effective dose of NMD670 and the active agent at the same time to the patient. In other exemplary embodiments, the therapeutically effective dose of NMD670 and the active agent are administered at different times to the patient. In some embodiments, the therapeutically effective dose of NMD670 and the active agent are administered sequentially.

In one aspect, the present disclosure relates to a method of treating myasthenia gravis that results in an improvement of a quantitative myasthenia gravis total score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cmax in the range of 5,000 ng/mL to 14,000 ng/mL in the patient, wherein after administration of the therapeutically effective dose of the compound, the patient experiences at least a 0.9 point reduction in their quantitative myasthenia gravis score when compared to placebo at the same time point.

In exemplary embodiments, the therapeutically effective dose of the compound further provides an AUC_{inf} the range of 15,000 ng/mL to 500,000 ng/mL in the patient. In exemplary embodiments, the therapeutically effective dose of the compound has a Tₘₐₓ in the patient ranging from 1 to 5 hours. In exemplary embodiments, the therapeutically effective dose of the compound has a half-life in the patient ranging from 3 hours to 7 hours. In exemplary embodiments, the therapeutically effective dose of the compound is administered orally to the patient. In exemplary embodiments, the patient experiences at least a 1.0 point reduction, such as at least 1.5 point reduction, such as at least 2.0 point reduction in their quantitative myasthenia gravis score. In exemplary embodiments, the Myasthenia Gravis Foundation of America (MGFA) clinical classification of the patient is class I, IIa, IIb IIIa, IIIb, IVa, IVb or V prior to treatment.

In one aspect, the present disclosure relates to a method of treating myasthenia gravis that results in an improvement of right-hand grip strength in a patient in need thereof, the method comprising administering a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 5,000 ng/mL to 14,000 ng/mL in the patient, wherein after administration of the therapeutically effective dose of the compound, the patient experiences at least a 1 kg improvement in their hand grip strength when compared to their hand grip strength before administration.

In exemplary embodiments, the at least 1 kg improvement in the patient's hand grip strength occurs at least 3 hours after administration of the therapeutically effective dose of the compound. In exemplary embodiments, the therapeutically effective dose of the compound further provides an AUC_{inf} in the range of 15,000 h·ng/mL to 500,000 h·ng/mL in the patient. In exemplary embodiments, the therapeutically effective dose of the compound has a Tₘₐₓ in the patient ranging from 1 to 5 hours. In exemplary embodiments, the therapeutically effective dose of the compound has a half-life in the patient ranging from 3 hours to 7 hours.

In one aspect, the present disclosure relates to a method of treating myasthenia gravis that results in a decrease in the Quantitative Myasthenia Gravis (QMG) total score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Quantitative Myasthenia Gravis (QMG) total score.

In exemplary embodiments, the subject or group of subjects experience a decrease in the Quantitative Myasthenia Gravis (QMG) total score after treatment with NMD670, wherein the score has decreased by at least 0.9 points, such as at least 1.0 point, such as at least 1.5 points, such as at least 2.0 points, such as at least 3.0 points.

In one aspect, the present disclosure relates to a method of treating myasthenia gravis that results in a decrease in the MG activities of daily living profile (MG-ADL) score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the MG activities of daily living profile (MG-ADL) score.

In exemplary embodiments, the subject experiences a decrease in the MG-ADL score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In one aspect, the present disclosure relates to a method of treating myasthenia gravis that results in an increase in muscle strength in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in muscle strength.

In exemplary embodiments the subject or group of subjects experience an increase in muscle strength after treatment with NMD670 when determined by measuring grip strength using a handheld dynamometer, wherein muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.

In exemplary embodiments the subject or group of subjects experience an increase in muscle strength after treatment with NMD670 when determined by measuring hand grip strength using a handheld dynamometer, wherein hand grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.

In one aspect, the present disclosure relates to a method of treating myasthenia gravis that results in a decrease in the Myasthenia Gravis Composite (MGC) scale in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Myasthenia Gravis Composite (MGC) scale.

In exemplary embodiments, the subject experiences a decrease in the MGC scale after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In one aspect, the present disclosure relates to a method of treating myasthenia gravis that results in a decrease in the Myasthenia Gravis Quality of Life 15 (MG-QOL15) score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Myasthenia Gravis Quality of Life 15 (MG-QOL15) score.

In exemplary embodiments, the subject experiences a decrease in the MG-QOL15 score after treatment with NMD670, wherein the score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In one aspect, the present disclosure relates to a method of treating myasthenia gravis that results in an increase in health state when determined using the EQ-5D scale in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an increase in health state when determined using the EQ-5D scale.

In exemplary embodiments, the subject experiences an increase in the EQ-5D scale after treatment with NMD670, wherein the score has increased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.

In one aspect, the present disclosure relates to a method of treating myasthenia gravis that results in a reduction in jitter in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a reduction in jitter.

In exemplary embodiments the subject experiences a reduction in jitter after treatment with NMD670 when determined using single fibre electromyography, wherein jitter has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%. In exemplary embodiments the subject experiences a reduction in jitter after treatment with NMD670 when determined using single fibre electromyography, wherein jitter has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.

In one aspect, the present disclosure relates to a method of treating myasthenia gravis that results in a reduction in blocking in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a reduction in blocking.

exemplary embodiments the subject experiences a reduction in blocking after treatment with NMD670 when determined using single fibre electromyography, wherein blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In one aspect, the present disclosure relates to a method of treating myasthenia gravis that results in a decrease in the Individualised Neuromuscular Quality of Life score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Individualised Neuromuscular Quality of Life score.

In exemplary embodiments, the subject experiences a decrease in the Individualised Neuromuscular Quality of Life score after treatment with NMD670, wherein the score has decreased 0.5 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In one aspect, the present disclosure relates to a method of treating myasthenia gravis that results in a decrease in the Fatigue Severity Scale score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences a decrease in the Fatigue Severity Scale score.

In exemplary embodiments, the subject or group of subjects experience a decrease in the Fatigue Severity Scale score after treatment with NMD670, wherein the score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.

In one aspect, the present disclosure relates to a method of treating myasthenia gravis that results in an improvement in pulmonary function in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mg to 1500 mg and provides a Cₘₐₓ in the range of 2,790 ng/mL to 76,700 ng/mL in the patient, wherein after administration of the therapeutically effective dose of the compound, the patient experiences an improvement in pulmonary function.

In exemplary embodiments, the subject or group of subjects experience an improvement in pulmonary function after treatment with NMD670 when determined by measuring forced vital capacity (FVC), wherein FVC has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the subject or group of subjects experience an improvement in pulmonary function after treatment with NMD670 when determined by measuring forced expiratory volume in 1 second (FEV1), wherein FEV1 has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the subject or group of subjects experience an improvement in pulmonary function after treatment with NMD670 when determined by measuring maximal inspiratory pressure (MIP), wherein MIP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the subject or group of subjects experience an improvement in pulmonary function after treatment with NMD670 when determined by measuring maximal expiratory pressure (MEP), wherein MEP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.

In exemplary embodiments, the therapeutically effective dose of the compound is administered orally to the patient. In exemplary embodiments, the therapeutically effective dose is within the range of 100 mgs to 600 mgs. In exemplary embodiments, the therapeutically effective dose is within the range of 200 mgs to 600 mgs. In exemplary embodiments, the therapeutically effective dose is 100 mg. In exemplary embodiments, the therapeutically effective dose is 150 mg. In exemplary embodiments the therapeutically effective dose is 200 mg. In exemplary embodiments, the therapeutically effective dose is 250 mg. In exemplary embodiments, the therapeutically effective dose is 300 mg. In exemplary embodiments, the therapeutically effective dose is 350 mg. In exemplary embodiments, the therapeutically effective dose is 400 mgs. In exemplary embodiments, the therapeutically effective dose is 500 mg. In exemplary embodiments, the therapeutically effective dose is 600 mg. In exemplary embodiments, the therapeutically effective dose is administered once, twice, three times or four times daily.

In one aspect, the present disclosure relates to a method for treating a patient suffering from symptoms of myasthenia gravis, the method comprising administering a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mgs to 1500 mgs.

In one aspect, the present disclosure relates to the use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of myasthenia gravis in a subject, wherein the composition is for administration at a therapeutic dose of 100 to 1500 mg of (2S)-2-[4-bromo-2-(1,2-oxazol-3-yl)phenoxy]propanoic acid.

The methods of the present disclosure may further comprise administering a second therapeutically effective dose of NMD670 to the patient either one day, two days, three days, four days, five days, six days or at least seven days after a first therapeutically effective dose is administered. The second therapeutically effective dose of NMD670 can range from 100 mgs to about 1500 mgs. In exemplary embodiments, the second therapeutically effective dose of NMD670 is any dosage disclosed herein. In other embodiments, the second therapeutically effective dose of NMD670 is the same as the first therapeutically effective dose administered to the patient.

In other exemplary embodiments, the methods of the present disclosure further comprise administering a third therapeutically effective dose of NMD670 to the patient either one day, two days, three days, four days, five days, six days or at least seven days after the second therapeutically effective dose has been administered. The third therapeutically effective dose of NMD670 can range from 100 mgs to about 1500 mgs.

In exemplary embodiments, the third therapeutically effective dose of NMD670 is any dosage disclosed herein. In other embodiments, the third therapeutically effective dose of NMD670 is the same as the first therapeutically effective dose and/or the second therapeutically effective dose administered to the patient.

In some exemplary embodiments, the administration of the therapeutically effective doses of NMD670 are repeated at least 1, 2, 3, 4, 5 or 6 times weekly. In other exemplary embodiments, the administration is repeated at least 1-3 times weekly, 2-5 times weekly or 3-6 times weekly.

In some exemplary embodiments, the administration of the therapeutically effective doses of NMD670 are repeated daily. The administration of the therapeutically effective doses of NMD670 may for example be repeated 1, 2, 3, 4, 5, 6, 7 or 8 times daily. In other embodiments, the administration is repeated 1 to 8 times daily or 2 to 5 times daily.

In some embodiments, the therapeutically effective dose of NMD670 is administered at least one time daily. In exemplary embodiments, the therapeutically effective dose of NMD670 is administered one time daily.

In other embodiments, the therapeutically effective dose of NMD670 is administered either two times daily, three times daily, or four times daily.

In exemplary embodiments, the therapeutically effective dose of NMD670 is 100 to 600 mg, 300 to 500 mg, or about 400 mg and is administered one time daily. In exemplary embodiments, the therapeutically effective dose of NMD670 is 200 to 600 mg, 300 to 500 mg, or about 400 mg and is administered one time daily.

In other exemplary embodiments, the therapeutically effective dose of NMD670 is 100 to 600 mg, 300 to 500 mg, or about 400 mg and is administered two times daily. In other exemplary embodiments, the therapeutically effective dose of NMD670 is 200 to 600 mg, 300 to 500 mg, or about 400 mg and is administered two times daily.

In some exemplary embodiments, the therapeutically effective dose of NMD670 is the daily dosage amount of NMD670. In these embodiments, the daily dosage amount of NMD670 can either be administered as a single dosage or can be administered in smaller dosages throughout the day. That is, in some embodiments the daily dosage of NMD670 is administered either once a day or at least one time daily, administered twice a day or at least at two different time points throughout the day, or administered three times a day or at least at three different time points throughout the day.

In other exemplary embodiments, the patient being administered the therapeutically effective dose of NMD670 suffers from ocular myasthenia gravis, early-onset generalised myasthenia gravis, late-onset generalised myasthenia gravis, generalized myasthenia gravis, seropositive myasthenia gravis, seronegative myasthenia gravis, AChR antibody positive myasthenia gravis, or muscle-specific kinase antibody-positive myasthenia gravis (MuSK-MG).

In some exemplary embodiments, the patient being administered the therapeutically effective dose of NMD670 suffers from Myasthenia Gravis Foundation of America (MGFA) class I, IIa, IIb, Ilila, IIIb, IVa, IVb, V symptoms or any combination thereof prior to treatment.

In other exemplary embodiments, the patient being administered the therapeutically effective dose of NMD670 does not have hyperuricemia. For example, the patient being administered the therapeutically effective dose of NMD670 has a level of serum uric acid below 6.5 mg/dL. Patients with serum uric acid levels above 6.5 mg/dL may not be suitable to receive the therapeutically effective dose. In exemplary embodiments wherein the patient possesses a serum uric acid level above 6.5 mg/dL, the treatment method may further comprise a step of administering a low dose of NMD670 until the patient's serum uric acid level falls below 6.5 mg/dL. A low dose of NMD670 can be from 20 mg to 150 mg, such as 25 mg to 100 mg, such as 25 mg to 50 mg. Once the patient's serum uric acid level falls below 6.5 mg/dL, they can begin receiving the therapeutically effective dose of NMD670.

### Kit of Parts

In one aspect, the present invention relates to a kit-of-parts comprising:
(2S)-2-[4-bromo-2-(1,2-oxazol-3-yl)phenoxy]propanoic acid, or a pharmaceutically acceptable salt, hydrate, polymorph, tautomer, or solvate thereof, and
an acetylcholinesterase inhibitor.

In exemplary embodiments, the kit-of-parts comprises 100 to 1500 mg of (2S)-2-[4-bromo-2-(1,2-oxazol-3-yl)phenoxy]propanoic acid, or a pharmaceutically acceptable salt, hydrate, polymorph, tautomer, or solvate thereof. In one embodiment, the acetylcholinesterase inhibitor is pyridostigmine.

In one aspect, the present invention relates to (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase for use in the treatment of myasthenia gravis.

In one aspect, the present invention relates to a method for treatment of myasthenia gravis comprising administering (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor to a subject in need thereof.

In one aspect, the kit-of-parts is for use in a method of treatment of myasthenia gravis in a subject.

In one aspect, the present invention relates to use of a kit-of-parts or a composition comprising (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor for the manufacture of a medicament for the treatment of myasthenia gravis.

In exemplary embodiments, the active agent is an acetylcholine esterase inhibitor. Said acetylcholine esterase inhibitor may, for example, be delta-9-tetrahydrocannabinol, carbamates, physostigmine, neostigmine, pyridostigmine, ambenonium, demecarium, rivastigmine, phenanthrene derivatives, galantamine, caffeine—noncompetitive, piperidines, donepezil, tacrine, edrophonium, huperzine, ladostigil, ungeremine, lactucopicrin or any combination thereof.

In other exemplary embodiments, the active agent may be an immunosuppressive drug. Immunosuppressive drugs are drugs that suppress or reduce the strength of the body's immune system. They are also known as anti-rejection drugs.

Immunosuppressive drugs that may be administered to the patient include, but are not limited to, glucocorticoids, corticosteroids, cytostatics, antibodies, drugs acting on immunophilins or any combination thereof. In exemplary embodiments, the active agent is prednisone.

In some exemplary embodiments, the active agent is an agent that is used in anti-myotonic treatment. Such agents can, for example, be blockers of voltage gated Na⁺ channels and aminoglycosides.

In some exemplary embodiments, the active agent is an agent for increasing the Ca²⁺ sensitivity of the contractile filaments in muscle. Such an agent can, for example, be tirasemtiv.

In other exemplary embodiments, the active agent is an agent for increasing ACh release that blocks voltage gated K⁺ channels in the pre-synaptic terminal. Such an agent can, for example, be 3,4-aminopyridine.

In some exemplary embodiments, the patient can be administered a therapeutically effective dose of NMD670 to treat or alleviate myasthenia gravis symptoms of double vision, ptosis, dysarthia or any combination.

### Items

1. A composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein the composition is for administration at a therapeutic dose of 100 to 1500 mg of (2S)-2-[4-bromo-2-(1,2-oxazol-3-yl)phenoxy]propanoic acid.
2. The composition for use according to item 1, wherein the therapeutic dose is less than 1500 mg, such as less than 1450 mg, such as less than 1300 mg, such as less than 1250 mg, such as less than 1200 mg, such as less than 1150 mg, such as less than 1100 mg, such as less than 1050 mg, such as less than 1000 mg, such as less than 950 mg, such as less than 900 mg, such as less than 850 mg, such as less than 800 mg, such as less than 750 mg, such as less than 700 mg, such as less than 650 mg, such as less than 600 mg, such as less than 550 mg, such as less than 500 mg, such as less than 450 mg, such as less than 400 mg, such as less than 350 mg, such as less than 300 mg, such as less than 250 mg.
3. The composition for use according to any one of the preceding items, wherein the therapeutic dose is at least 100 mg, such as at least 150 mg, such as at least 200 mg, such as at least 250 mg, such as at least 300 mg, such as at least 350 mg, such as at least 400 mg, such as at least 450 mg, such as at least 500 mg, such as at least 550 mg, such as at least 600 mg, such as at least 650 mg, such as at least 700 mg, such as at least 750 mg, such as at least 800 mg, such as at least 850 mg, such as at least 900 mg, such as at least 950 mg, such as at least 1000 mg, such as at least 1050 mg, such as at least 1100 mg, such as at least 1150 mg, such as at least 1200 mg, such as at least 1250 mg, such as at least 1300 mg, such as at least 1350 mg, such as at least 1400 mg, such as at least 1450 mg.
4. The composition for use according to item 1, wherein the therapeutic dose is 200 to 600 mg, such as 250 to 550 mg, such as 300 to 500 mg, such as 350 to 450 mg, such as 375 to 425 mg, such as 400 mg.
5. The composition for use according to item 1, wherein the therapeutic dose is 700 to 1400 mg, such as 800 to 1350 mg, such as 900 to 1300 mg, such as 1000 to 1250 mg, such as 1100 to 1250 mg, such as about 1200 mg.
6. The composition for use according to item 1, wherein the therapeutic dose is about 100 mg.
7. The composition for use according to item 1, wherein the therapeutic dose is about 150 mg.
8. The composition for use according to item 1, wherein the therapeutic dose is about 200 mg.
9. The composition for use according to item 1, wherein the therapeutic dose is about 250 mg.
10. The composition for use according to item 1, wherein the therapeutic dose is about 300 mg.
11. The composition for use according to item 1, wherein the therapeutic dose is about 350 mg.
12. The composition for use according to item 1, wherein the therapeutic dose is about 400 mg.
13. The composition for use according to item 1, wherein the therapeutic dose is about 500 mg.
14. The composition for use according to item 1, wherein the therapeutic dose is about 600 mg.
15. The composition for use according to any one of the preceding items, wherein the therapeutic dose is to be administered at least one time daily.
16. The composition for use according to any one of the preceding items, wherein the therapeutic dose is to be administered one time daily.
17. The composition for use according to any one of items 1 to 14, wherein the therapeutic dose is to be administered two times daily.
18. The composition for use according to any one of items 1 to 14, wherein the therapeutic dose is to be administered three times daily.
19. The composition for use according to any one of items 1 to 14, wherein the therapeutic dose is to be administered four times daily.
20. The composition for use according to item 1, wherein the therapeutic dose is 100 to 600 mg and the composition is to be administered one time daily.
21. The composition for use according to item 1, wherein the therapeutic dose is 200 to 600 mg and the composition is to be administered one time daily.
22. The composition for use according to item 1, wherein the therapeutic dose is 300 to 500 mg and the composition is to be administered one time daily.
23. The composition for use according to item 1, wherein the therapeutic dose is about 100 mg and the composition is to be administered one time daily.
24. The composition for use according to item 1, wherein the therapeutic dose is about 150 mg and the composition is to be administered one time daily.
25. The composition for use according to item 1, wherein the therapeutic dose is about 200 mg and the composition is to be administered one time daily.
26. The composition for use according to item 1, wherein the therapeutic dose is about 250 mg and the composition is to be administered one time daily.
27. The composition for use according to item 1, wherein the therapeutic dose is about 300 mg and the composition is to be administered one time daily.
28. The composition for use according to item 1, wherein the therapeutic dose is about 350 mg and the composition is to be administered one time daily.
29. The composition for use according to item 1, wherein the therapeutic dose is about 400 mg and the composition is to be administered one time daily.
30. The composition for use according to item 1, wherein the therapeutic dose is about 500 mg and the composition is to be administered one time daily.
31. The composition for use according to item 1, wherein the therapeutic dose is about 600 mg and the composition is to be administered one time daily.
32. The composition for use according to item 1, wherein the therapeutic dose is 100 to 600 mg and the composition is to be administered two times daily.
33. The composition for use according to item 1, wherein the therapeutic dose is 200 to 600 mg and the composition is to be administered two times daily.
34. The composition for use according to any one of the preceding items, wherein the therapeutic dose is 300 to 500 mg and the composition is to be administered two times daily. 35. The composition for use according to item 1, wherein the therapeutic dose is about 100 mg and the composition is to be administered two times daily.
36. The composition for use according to item 1, wherein the therapeutic dose is about 150 mg and the composition is to be administered two times daily.
37. The composition for use according to item 1, wherein the therapeutic dose is about 200 mg and the composition is to be administered two times daily.
38. The composition for use according to item 1, wherein the therapeutic dose is about 250 mg and the composition is to be administered two times daily.
39. The composition for use according to item 1, wherein the therapeutic dose is about 300 mg and the composition is to be administered two times daily.
40. The composition for use according to item 1, wherein the therapeutic dose is about 350 mg and the composition is to be administered two times daily.
41. The composition for use according to item 1, wherein the therapeutic dose is about 400 mg and the composition is to be administered two times daily.
42. The composition for use according to item 1, wherein the therapeutic dose is about 500 mg and the composition is to be administered two times daily.
43. The composition for use according to item 1, wherein the therapeutic dose is about 600 mg and the composition is to be administered two times daily.
44. The composition for use according to item 1, wherein the therapeutic dose is 100
to 600 mg and the composition is to be administered three times daily.
45. The composition for use according to item 1, wherein the therapeutic dose is 200 to 600 mg and the composition is to be administered three times daily.
46. The composition for use according to item 1, wherein the therapeutic dose is 300 to 500 mg and the composition is to be administered three times daily.
47. The composition for use according to item 1, wherein the therapeutic dose is about 100 mg and the composition is to be administered three times daily.
48. The composition for use according to item 1, wherein the therapeutic dose is about 150 mg and the composition is to be administered three times daily.
49. The composition for use according to item 1, wherein the therapeutic dose is about 200 mg and the composition is to be administered three times daily.
50. The composition for use according to item 1, wherein the therapeutic dose is about 250 mg and the composition is to be administered three times daily.
51. The composition for use according to item 1, wherein the therapeutic dose is about 300 mg and the composition is to be administered three times daily.
52. The composition for use according to item 1, wherein the therapeutic dose is about 350 mg and the composition is to be administered three times daily.
53. The composition for use according to item 1, wherein the therapeutic dose is about 400 mg and the composition is to be administered three times daily.
54. The composition for use according to item 1, wherein the therapeutic dose is about 500 mg and the composition is to be administered three times daily.
55. The composition for use according to item 1, wherein the therapeutic dose is about 600 mg and the composition is to be administered three times daily.
56. The composition for use according to item 1, wherein the therapeutic dose is 100 to 600 mg and the composition is to be administered four times daily.
57. The composition for use according to item 1, wherein the therapeutic dose is 200 to 600 mg and the composition is to be administered four times daily.
58. The composition for use according to any one of the preceding items, wherein the therapeutic dose is the total daily dosage.
59. The composition for use according to any one of the preceding items, wherein the composition is administered orally, parenterally, intravenously, inhaled, topically, enterally, rectally, buccally or as an aerosol.
60. The composition for use according to any one of items 1 to 59, wherein the composition is in the form of a liquid, liquid suspension, oil, emulsion, or syrup.
61. The composition for use according to any one of items 1 to 59, wherein the composition is a solid dosage form.
62. The composition for use according to item 61, wherein the solid dosage form is administered orally.
63. The composition for use according to any one of items 1 to 62, wherein the composition is in the form of a solid dosage form and is to be dosed orally, the therapeutic dose is 100 to 600 mg and the composition is to be administered one time daily.
64. The composition for use according to any one of items 1 to 62, wherein the composition is in the form of a solid dosage form and is to be dosed orally, the therapeutic dose is 200 to 600 mg and the composition is to be administered one time daily.
65. The composition for use according to any one of items 1 to 62, wherein the composition is in the form of a solid dosage form and is to be dosed orally, the therapeutic dose is 100 to 600 mg and the composition is to be administered two times daily.
66. The composition for use according to any one of items 1 to 62, wherein the composition is in the form of a solid dosage form and is to be dosed orally, the therapeutic dose is 200 to 600 mg and the composition is to be administered two times daily.
67. The composition for use according to any one of items 1 to 62, wherein the composition is in the form of a solid dosage form and is to be dosed orally, the therapeutic dose is 100 to 600 mg and the composition is to be administered three times daily.
68. The composition for use according to any one of items 1 to 62, wherein the composition is in the form of a solid dosage form and is to be dosed orally, the therapeutic dose is 200 to 600 mg and the composition is to be administered three times daily.
69. The composition for use according to any one of items 1 to 62, wherein the composition is in the form of a solid dosage form and is to be dosed orally, the therapeutic dose is 100 to 600 mg and the composition is to be administered four times daily.
70. The composition for use according to any one of items 1 to 62, wherein the composition is in the form of a solid dosage form and is to be dosed orally, the therapeutic dose is 200 to 600 mg and the composition is to be administered four times daily.
71. The composition for use according to any of items 61 to 70, wherein the solid dosage form is selected from the group consisting of capsule (such as sprinkle capsule and gelatine capsule), tablet (such as uncoated tablet, coated tablet, slow-release tablet) and sprinkle.
72. The composition for use according to any of items 61 to 71, wherein the solid dosage form releases not less than 80% of the compound after 30 minutes, when measured in a United States Pharmacopeia (USP) type 2 dissolution apparatus, paddle at 75 rpm, at a temperature of 37° C.±0.5° C. in 900 mL of pH 6.8 phosphate/citric acid buffer.
73. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 5 hours, such 1.5 to 4 hours, such as about 2 hours or about 3 hours, after administration.
74. The composition for use according to any one of items 1 to 72, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 6 hours after administration.
75. The composition for use according to any one of items 1 to 72, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 3 to 7 hours after administration.
76. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 13,000 to 32,000 ng/mL, such as 15,000 to 30,000 ng/mL, such as 15,730 to 27,670 ng/mL, such as 16,000 to 27,000 ng/mL, such as 17,360 to 27,125 ng/mL, such as 18,000 to 25,000 ng/mL, such as 20,000 to 23,000 ng/mL, such as about 21,700 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
77. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 21,700 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
78. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 40,000 to 110,000 ng/mL, such as 46,300 to 107,100 ng/mL, such as 50,000 to 100,000 ng/mL, such as 60,000 to 90,000 ng/mL, such as 61,360 to 95,875 ng/mL, such as 70,000 to 80,000 ng/mL, such as about 76,700 ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof.
79. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is about 80% to about 125%, such as 80.00% to 125.00%, of 76,700 ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof.
80. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 60,000 to 130,000 h·ng/mL, such as 64,100 to 123,300 h·ng/mL, such as 70,000 to 120,000 h·ng/mL, such as 74,960 to 117,125 h·ng/mL, such as 80,000 to 110,000 h·ng/mL, such as 90,000 to 100,000 h·ng/mL, such as about 93,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
81. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 93,700 h·ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.
82. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is 250,000 to 500,000 h·ng/mL, such as 265,000 to 491,000 h·ng/mL, such as 280,000 to 480,000 h·ng/mL, such as 302,400 to 472,500 h·ng/mL, such as 320,000 to 450,000 h·ng/mL, such as 340,000 to 425,000 h·ng/mL, such as 360,000 to 400,000 h·ng/mL, such as about 378,300 h·ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof.
83. The composition for use according to any one of the preceding items, wherein the composition is to be dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean AUC_{0-infinity} is about 80% to about 125%, such as 80.00% to 125.00%, of 378,300 h·ng/mL after administration with a single dose of 1200 mg (2S)-2-[4-bromo-2-(1,2-oxazol-3-yl)phenoxy]propanoic acid, or a pharmaceutically acceptable salt, hydrate, polymorph, tautomer, or solvate thereof.
84. The composition for use according to any of the preceding items, wherein said AUC_{0-24infinity}, Cₘₐₓ or Tₘₐₓ is measured after administration of a single dose to a human subject suffering from myasthenia gravis.
85. The composition for use according to any one of the preceding items, wherein the composition further comprises at least one pharmaceutically acceptable adjuvant and/or excipient.
86. The composition for use according to item 85, wherein the pharmaceutically acceptable adjuvant and/or excipient is selected from the group consisting of filler, binder, lubricant and disintegrant.
87. The composition for use according to item 85, wherein the pharmaceutically acceptable adjuvant and/or excipient is selected from the group consisting of silicified microcrystalline cellulose, microcrystalline cellulose, maltodextrin, magnesium stearate and croscarmellose sodium.
88. The composition for use according to any of items 61 to 87, wherein the composition comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.
89. The composition for use according to any of items 61 to 87, wherein the composition comprises 10 to 80 wt %, such as 40 to 65 wt %, such as 50 to 60 wt %, such as 50 to 55 wt %, such as 55 to 60 wt %, such as about 53 wt %, such as 56 wt % (2S)-2-[4-bromo-2-(1,2-oxazol-3-yl)phenoxy]propanoic acid, or a pharmaceutically acceptable salt, hydrate, polymorph, tautomer, or solvate thereof.
90. The composition for use according to any of items 61 to 87, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant; and
   e. 0.25 to 5 wt %, such as 0.3 to 2.5 wt % disintegrant;
with the proviso that the sum of the wt % of the components does not exceed 100 wt %.
91. The composition for use according to any of items 61 to 87, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate; and
   f. 0.25 to 5 wt %, such as 0.3 to 2.5 wt % Croscarmellose sodium;
with the proviso that the sum of the wt % of the components does not exceed 100 wt %.
92. The composition for use according to any of items 61 to 87, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant;
   e. 0.25 to 5 wt %, such as 0.3 to 2.5 wt % disintegrant; and
   f. 1 to 10 wt % film coating;
with the proviso that the sum of the wt % of the components does not exceed 100 wt %.
93. The composition for use according to any of items 61 to 87, wherein the composition comprises or consists of:
   a. 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white,
with the proviso that the sum of the wt % of the components does not exceed 100 wt %.
94. The composition for use according to any one of the preceding items, wherein the composition is administered as one or more unit dosage forms.
95. The composition for use according to any one of the preceding items, wherein the subject has a level of serum uric acid below 6.5 mg/dL.
96. The composition for use according to any one of the preceding items, wherein the Myasthenia Gravis Foundation of America (MGFA) clinical classification of the subject is class I, IIa, IIb IIIa, IIIb, IVa, IVb or V prior to treatment.
97. The composition for use according to item 96, wherein MGFA clinical classification is I, IIa, IIb, or IIIb prior to treatment.
98. The composition for use according to any one of the preceding items, wherein the subject experiences a reduction in Quantitative Myasthenia Gravis (QMG) total score after treatment.
99. The composition for use according to item 98, wherein the reduction in QMG total score after treatment is at least 0.9 points between 2 and 5 hours, such as 2 hours after treatment, such as 3 hours after treatment, such as 4 hours after treatment or such as 5 hours after treatment.
100. The composition for use according to any one of the preceding items, wherein the Quantitative Myasthenia Gravis (QMG) total score is reduced compared to placebo at the same time point after treatment.
101. The composition for use according to item 100, wherein the reduction in QMG total score compared to placebo at the same time point after treatment is at least 0.9 points between 2 and 5 hours, such as 2 hours after treatment, such as 3 hours after treatment, such as 4 hours after treatment or such as 5 hours after treatment.
102. The composition for use according to item 98, wherein the QMG total score is determined by comparing the change from baseline in the a decrease in the QMG total score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in a decrease in the QMG total score after a defined period of time of placebo treatment.
103. The composition for use according to item 102, wherein the period of time is 21 days.
104. The composition for use according to any one of items 98 to 103, wherein reduction in QMG total score is at least 1.0 point, such as at least 1.5 points, such as at least 2.0 points, such as at least 3.0 points.
105. The composition for use according to any one of the preceding items, wherein the subject or a group of subjects experience a decrease in the MG activities of daily living profile (MG-ADL) score after treatment with the composition.
106. The composition for use according to item 105, wherein the MG-ADL score is determined by comparing the change from baseline the MG-ADL score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the MG-ADL score after a defined period of time of placebo treatment.
107. The composition for use according to item 106, wherein the period of time is 21 days.
108. The composition for use according to any one of the preceding items, wherein the MG-ADL score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points.
109. The composition for use according to any one of the preceding items, wherein the MG-ADL score has decreased by between 0.3 and 10 points, such as between 0.5 and 6 points, such as between 0.3 and 5 points.
110. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a a decrease in the MG activities of daily living profile score.
111. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a a decrease in the MG activities of daily living profile score.
112. The composition for use according to any one of the preceding items, wherein the subject or a group of subjects experience an increase in muscle strength after treatment with the composition.
113. The composition for use according to item 112, wherein the strength is measured using a handheld dynamometer.
114. The composition for use according to any one of items 112 or 113, wherein the muscle strength is measured as grip strength.
115. The composition for use according to any one of items 112 or 113, wherein the muscle strength is measured as the strength of the thigh (knee flexors), the upper arm (elbow flexor and extension) and/or the shoulder (shoulder abduction).
116. The composition for use according to any one of items 112 to 115, wherein the increase muscle strength is determined by comparing the change from baseline in muscle strength after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in muscle strength after a defined period of time of placebo treatment.
117. The composition for use according to item 116, wherein the period of time is 21 days.
118. The composition for use according to any one of items 112 to 117, the muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
119. The composition for use according to any one of items 112 to 118, wherein the muscle strength has increased by between 10% and 400%, such as between 15% and 200%, such as between 20% and 100%.
120. The composition for use according to any one of items 112 to 119, the muscle strength determined by measuring grip strength using a handheld dynamometer has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
121. The composition for use according to any one of items 112 to 120, wherein the muscle strength determined by measuring grip strength using a handheld dynamometer has increased by between 10% and 400%, such as between 15% and 200%, such as between 20% and 100%.
122. The composition for use according to any one of items 112 to 121, wherein the muscle strength determined by measuring grip strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
123. The composition for use according to any one of items 112 to 122, wherein the muscle strength determined by measuring grip strength using a handheld dynamometer has increased by between 0.25 and 5.0 kg, such as between 0.25 and 4.0 kg, such as between 0.5 and 4.0 kg.
124. The composition for use according to any one of items 112 to 123, wherein the muscle strength determined by measuring knee flexor strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
125. The composition for use according to any one of items 112 to 124, wherein the muscle strength determined by measuring knee flexor strength using a handheld dynamometer has increased by between 0.25 and 5.0 kg, such as between 0.25 and 4.0 kg, such as between 0.5 and 4.0 kg.
126. The composition for use according to any one of items 112 to 125, wherein the muscle strength determined by measuring elbow flexor strength using a handheld dynamometer has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg.
127. The composition for use according to any one of items 112 to 126, wherein the muscle strength determined by measuring elbow flexor strength using a handheld dynamometer has increased by between 0.25 and 5.0 kg, such as between 0.25 and 4.0 kg, such as between 0.5 and 4.0 kg.
128. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in in muscle strength.
129. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an increase in in muscle strength.
130. The composition for use according to any one of the preceding items, wherein the subject or a group of subjects experience a decrease in the Myasthenia Gravis Composite (MGC) scale after treatment with the composition.
131. The composition for use according to item 130, wherein the decrease in the MGC scale is determined by comparing the change from baseline in the MGC scale after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the MGC scale after a defined period of time of placebo treatment.
132. The composition for use according to item 131, wherein the period of time is 21 days.
133. The composition for use according to any one of items 130 to 132, wherein the MGC scale has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points.
134. The composition for use according to any one of items 130 to 133, wherein the MGC scale has decreased by between has increased by between 0.3 and 10 points, such as between 0.5 and 6 points, such as between 0.3 and 5 points.
135. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Myasthenia Gravis Composite scale.
136. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Myasthenia Gravis Composite scale.
137. The composition for use according to any one of the preceding items, wherein the subject or a group of subjects experience a decrease in the Myasthenia Gravis Quality of Life 15 (MG-QOL15) score after treatment with the composition.
138. The composition for use according to item 137, wherein the decrease in the MG-QCL15 score is determined by comparing the change from baseline in the a decrease in the MG-QCL15 score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in a decrease in the MG-QCL15 score after a defined period of time of placebo treatment.
139. The composition for use according to item 138, wherein the period of time is 21 days.
140. The composition for use according to any one of items 137 to 139, wherein the MG-QCL15 score has decreased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points.
141. The composition for use according to any one of items 137 to 140, wherein the MG-QCL15 score has decreased by between has increased by between 0.3 and 10 points, such as between 0.5 and 6 points, such as between 0.3 and 5 points.
142. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Myasthenia Gravis Quality of Life 15 score.
143. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Myasthenia Gravis Quality of Life 15 score.
144. The composition for use according to any one of the preceding items, wherein the subject or a group of subjects experience an increase in health when determined using the EQ-5D scale after treatment with the composition.
145. The composition for use according to item 144, wherein the an increase in the EQ-5D scale is determined by comparing the change from baseline in the EQ-5D scale after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the EQ-5D scale after a defined period of time of placebo treatment.
146. The composition for use according to item 145, wherein the period of time is 21 days.
147. The composition for use according to any one of items 144 to 146, wherein the EQ-5D scale has increased by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points.
148. The composition for use according to any one of items 144 to 147, wherein the EQ-5D scale has increased by between has increased by between 0.3 and 10 points, such as between 0.5 and 6 points, such as between 0.3 and 5 points.
149. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences increase in health state when determined using the EQ-5D scale.
150. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences increase in health state when determined using the EQ-5D scale.
151. The composition for use according to any one of the preceding items, wherein the subject or a group of subjects experience a reduction in jitter after treatment with the composition.
152. The composition for use according to item 151, wherein the jitter is determined using single fibre electromyography.
153. The composition for use according to any one of items 151 or 152, wherein the reduction in jitter is determined by comparing the change from baseline in jitter after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in jitter after a defined period of time of placebo treatment.
154. The composition for use according to item 153, wherein the period of time is 21 days.
155. The composition for use according to any one items 151 to 154, wherein the jitter has been reduced by at least 10%, such as at least 15%, such as at least 20%, such as at least 25%, such as at least 30%, such as at least 50%, such as at least 75%, such as at least 100%, such as at least 150%, such as at least 200%.
156. The composition for use according to any one of items 151 to 154, wherein the jitter has been reduced by between 5% and 95%, such as between 5% and 80%, such as between 10% and 50%.
157. The composition for use according to any one items 151 to 154, wherein the jitter determined using single fibre electromyography has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs.
158. The composition for use according to any one items 151 to 154, wherein the jitter determined using single fibre electromyography has been reduced by between 5 μs and 200 μs, such as between 5 μs and 100 μs, such as between 10 μs and 50 μs.
159. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in jitter when determined using single fibre electromyography.
160. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in jitter when determined using single fibre electromyography.
161. The composition for use according to any one of the preceding items, wherein the subject or a group of subjects experience a reduction in blocking after treatment with the composition.
162. The composition for use according to item 161, wherein the blocking is determined using single fibre electromyography.
163. The composition for use according to any one of items 161 or 162, wherein the reduction in blocking is determined by comparing the change from baseline in blocking after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in blocking after a defined period of time of placebo treatment.
164. The composition for use according to item 163, wherein the period of time is 21 days.
165. The composition for use according to any one of items 161 to 164, wherein the blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%.
166. The composition for use according to any one of items 161 to 165, wherein the blocking has been reduced by between 5% and 95%, such as between 5% and 80%, such as between 10% and 50%.
167. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in blocking when determined using single fibre electromyography.
168. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a reduction in blocking when determined using single fibre electromyography.
169. The composition for use according to any one of the preceding items, wherein the subject or a group of subjects experience a decrease in the Individualised Neuromuscular Quality of Life score after treatment with the composition.
170. The composition for use according to item 169, wherein the decrease in the Individualised Neuromuscular Quality of Life score is determined by comparing the change from baseline in the Individualised Neuromuscular Quality of Life score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the Individualised Neuromuscular Quality of Life score after a defined period of time of placebo treatment.
171. The composition for use according to item 170, wherein the period of time is 21 days.
172. The composition for use according to any one of items 169 to 171, wherein the Individualised Neuromuscular Quality of Life score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
173. The composition for use according to any one of the preceding items, wherein the Individualised Neuromuscular Quality of Life score has decreased by between 0.5 and 30 points, such as between 1 and 20 points, such as between 0.5 and 10 points.
174. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Individualised Neuromuscular Quality of Life score.
175. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Individualised Neuromuscular Quality of Life score.
176. The composition for use according to any one of the preceding items, wherein the subject or a group of subjects experience a decrease in the Fatigue Severity Scale score after treatment with the composition.
177. The composition for use according to item 176, wherein the decrease in the Fatigue Severity Scale score is determined by comparing the change from baseline in the Fatigue Severity Scale score after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline in the Fatigue Severity Scale score after a defined period of time of placebo treatment.
178. The composition for use according to item 177, wherein the period of time is 21 days.
179. The composition for use according to any one of items 176 to 178, wherein the Fatigue Severity Scale score has decreased by 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points.
180. The composition for use according to any one of items 176 to 179, wherein the Fatigue Severity Scale score has decreased by between 1 and 20 points, such as between 0.5 and 10 points.
181. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Fatigue Severity Scale score.
182. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences a decrease in the Fatigue Severity Scale score.
183. The composition for use according to any one of the preceding items, wherein the subject or a group of subjects experience an improvement in pulmonary function after treatment with the composition.
184. The composition for use according to item 183, wherein the an improvement in pulmonary function is determined by comparing the change from baseline of the pulmonary function after a defined period of time treatment with the composition comprising (2S)-2-[4-bromo-2-(1,2-oxazol-3-yl)phenoxy]propanoic acid, or a pharmaceutically acceptable salt, hydrate, polymorph, tautomer, or solvate thereof, with the change from baseline of the pulmonary function after a defined period of time of placebo treatment.
185. The composition for use according to item 184, wherein the period of time is 21 days.
186. The composition for use according to any one of items 183 to 185, wherein the pulmonary function is determined by measuring forced vital capacity (FVC).
187. The composition for use according to item 186, wherein the FVC has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
188. The composition for use according to item 186, wherein the FVC has increased by between 5% and 95%, such as between 5% and 80%, such as between 10% and 50%.
189. The composition for use according to any one of items 183 to 185, wherein the pulmonary function is determined by measuring forced expiratory volume in 1 second (FEV1).
190. The composition for use according to item 189, wherein FEV1 has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
191. The composition for use according to item 189, wherein FEV1 has increased by between 5% and 95%, such as between 5% and 80%, such as between 10% and 50%.
192. The composition for use according to any one of items 183 to 185, wherein the pulmonary function is determined by measuring maximal inspiratory pressure (MIP).
193. The composition for use according to item 192, wherein the MIP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
194. The composition for use according to item 192, wherein the MIP has increased by between 5% and 95%, such as between 5% and 80%, such as between 10% and 50%.
195. The composition for use according to any one of items 183 to 185, wherein the pulmonary function is determined by measuring maximal expiratory pressure (MEP).
196. The composition for use according to item 195, wherein the MEP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%.
197. The composition for use according to item 195, wherein the MEP has increased by between 5% and 95%, such as between 5% and 80%, such as between 10% and 50%.
198. The composition for use according to any one of the preceding items, wherein the composition is for administration at a therapeutic dose of 100 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in pulmonary function.
199. The composition for use according to any one of the preceding items, wherein the composition is for administration at a therapeutic dose of 200 to 600 mg of (2S)-2-[4-bromo-2-(1,2-oxazol-3-yl)phenoxy]propanoic acid; the composition is to be administered two times daily; the composition is in the form of a solid dosage form and is to be administered orally; and the subject experiences an improvement in pulmonary function.
200. A composition comprising (2S)-2-[4-bromo-2-(1,2-oxazol-3-yl)phenoxy]propanoic acid, or a pharmaceutically acceptable salt, hydrate, polymorph, tautomer, or solvate thereof, for use in a method of treatment of myasthenia gravis in a subject, wherein the subject has a level of serum uric acid below 6.5 mg/dL.
201. The composition for use according to item 200, wherein the composition is for administration at a therapeutic dose of 100 to 1500 mg of (2S)-2-[4-bromo-2-(1,2-oxazol-3-yl)phenoxy]propanoic acid.
202. The composition for use according to any one of items 200 to 201, wherein the therapeutic dose is as defined in any one of items 2 to 57.
203. A composition, formulated as a solid dosage form, comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition comprises 50 to 400 mg (2S)-2-[4-bromo-2-(1,2-oxazol-3-yl)phenoxy]propanoic acid.
204. The composition according to item 203, wherein the solid dosage form comprises 100 mg of (2S)-2-[4-bromo-2-(1,2-oxazol-3-yl)phenoxy]propanoic acid, or a pharmaceutically acceptable salt, hydrate, polymorph, tautomer, or solvate thereof.
205. The composition according to item 203, wherein the solid dosage form comprises 150 mg of (2S)-2-[4-bromo-2-(1,2-oxazol-3-yl)phenoxy]propanoic acid, or a pharmaceutically acceptable salt, hydrate, polymorph, tautomer, or solvate thereof.
206. The composition according to item 203, wherein the solid dosage form comprises 200 mg of (2S)-2-[4-bromo-2-(1,2-oxazol-3-yl)phenoxy]propanoic acid, or a pharmaceutically acceptable salt, hydrate, polymorph, tautomer, or solvate thereof.
207. The composition according to item 203, wherein the solid dosage form comprises 250 mg of (2S)-2-[4-bromo-2-(1,2-oxazol-3-yl)phenoxy]propanoic acid, or a pharmaceutically acceptable salt, hydrate, polymorph, tautomer, or solvate thereof.
208. The composition according to item 203, wherein the solid dosage form comprises 300 mg of (2S)-2-[4-bromo-2-(1,2-oxazol-3-yl)phenoxy]propanoic acid, or a pharmaceutically acceptable salt, hydrate, polymorph, tautomer, or solvate thereof.
209. The composition according to item 203, wherein the solid dosage form comprises 350 mg of (2S)-2-[4-bromo-2-(1,2-oxazol-3-yl)phenoxy]propanoic acid, or a pharmaceutically acceptable salt, hydrate, polymorph, tautomer, or solvate thereof.
210. The composition according to item 203, wherein the solid dosage form comprises 400 mg of (2S)-2-[4-bromo-2-(1,2-oxazol-3-yl)phenoxy]propanoic acid, or a pharmaceutically acceptable salt, hydrate, polymorph, tautomer, or solvate thereof.
211. The composition according to any one of items 203 to 209, wherein the composition further comprises at least one pharmaceutically acceptable adjuvant and/or excipient.
212. The composition according to any of items 203 to 211, wherein the composition is administered orally.
213. The composition according to any of items 203 to 212, wherein the solid dosage form is selected from the group consisting of capsule (such as sprinkle capsule and gelatine capsule), tablet (such as uncoated tablet, coated tablet, slow-release tablet) and sprinkle.
214. The composition according to any of items 211 to 213, wherein the pharmaceutically acceptable adjuvant and/or excipient is selected from the group consisting of filler, binder, lubricant and disintegrant.
215. The composition according to any of items 211 to 213, wherein the pharmaceutically acceptable adjuvant and/or excipient is selected from the group consisting of silicified microcrystalline cellulose, microcrystalline cellulose, maltodextrin, magnesium stearate and croscarmellose sodium.
216. The composition according to any of items 203 to 215, wherein the composition comprises 10 to 80 wt %, such as 40 to 65 wt %, such as 50 to 55 wt %, such as about 53 wt % (2S)-2-[4-bromo-2-(1,2-oxazol-3-yl)phenoxy]propanoic acid, or a pharmaceutically acceptable salt, hydrate, polymorph, tautomer, or solvate thereof.
217. The composition according to any of items 203 to 215, wherein the composition comprises 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof.
218. The composition according to any of items 203 to 215, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant; and
   e. 0.25 to 5 wt %, such as 0.3 to 2.5 wt % disintegrant;
with the proviso that the sum of the wt % of the components does not exceed 100 wt %.
219. The composition according to any of items 203 to 215, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate; and
   f. 0.25 to 5 wt %, such as 0.3 to 2.5 wt % Croscarmellose sodium;
with the proviso that the sum of the wt % of the components does not exceed 100 wt %.
220. The composition according to any of items 203 to 215, wherein the composition comprises:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 20 to 80 wt %, such as 25 to 50 wt % filler;
   c. 2 to 20 wt %, such as 3 to 16 wt % binder;
   d. 0.25 to 3 wt %, such as 0.4 to 2.0 wt % lubricant;
   e. 0.25 to 5 wt %, such as 0.3 to 2.5 wt % disintegrant; and
   f. 1 to 10 wt % film coating;
with the proviso that the sum of the wt % of the components does not exceed 100 wt %.
221. The composition according to any of items 203 to 215, wherein the composition comprises or consists of:
   a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
   b. 5 to 60 wt %, such as 20 to 40 wt %, such as 21 to 37 wt % silicified microcrystalline cellulose;
   c. 2 to 60 wt %, such as 5 to 16 wt % microcrystalline cellulose;
   d. 1 to 15 wt %, such as 1.5 to 7 wt %, such as 1.8 to 6.0 wt % maltodextrin;
   e. 0.25 to 3 wt %, such as 0.4 to 2.0 wt % magnesium stearate;
   f. 0.25 to 5 wt %, such as 0.3 to 2.5 wt % Croscarmellose sodium; and
   g. 1 to 10 wt % film coating composition such as Opadry white,
with the proviso that the sum of the wt % of the components does not exceed 100 wt %.
222. A composition, formulated as a solid dosage form, comprising a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof,
wherein the therapeutically effective dose is within the range of 50 mgs to 400 mgs.
223. The composition of item 222, wherein the therapeutically effective dose is 400 mgs.
224. The composition of item 222, wherein the therapeutically effective dose is 350 mgs.
225. The composition of item 222, wherein the therapeutically effective dose is 300
mgs.
226. The composition of item 222, wherein the therapeutically effective dose is 250 mgs.
227. The composition of item 222, wherein the therapeutically effective dose is 200 mgs.
228. The composition of item 222, wherein the therapeutically effective dose is 150 mgs.
229. The composition of item 222, wherein the therapeutically effective dose is 100 mgs.
230. The composition of item 222, wherein the therapeutically effective dose is given once daily.
231. The composition of item 222, wherein the therapeutically effective dose is given twice daily.
232. The composition of item 222, wherein the therapeutically effective dose is given three times daily.
233. The composition according to any one of items 203 to 232, wherein the composition is for oral administration.
234. A method for treatment of myasthenia gravis in a subject in need thereof, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, at a therapeutic dose of 100 to 1500 mg of (2S)-2-[4-bromo-2-(1,2-oxazol-3-yl)phenoxy]propanoic acid to said subject.
235. The method according to item 234, wherein the method results in:
   a. a reduction in Quantitative Myasthenia Gravis total score;
   b. a decrease in the MG activities of daily living profile (MG-ADL) score;
   c. an increase in muscle strength;
   d. a decrease in the Myasthenia Gravis Composite (MGC) scale;
   e. a decrease in the Myasthenia Gravis Quality of Life 15 (MG-QOL15) score;
   f. an increase in health state when determined using the EQ-5D scale;
   g. a reduction in jitter;
   h. a reduction in blocking;
   i. a decrease in the Individualised Neuromuscular Quality of Life score;
   j. a decrease in the Fatigue Severity Scale score; and/or
   k. an improvement in pulmonary function.
236. A method for treating myasthenia gravis in a subject that result in a decrease in the MG activities of daily living profile (MG-ADL) score, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
237. The method according to item 236, wherein the therapeutically effective dose provides a Cₘₐₓ in the range of 2,790 ng/mL to 76,700 ng/mL in the subject.
238. The method according to one of items 236 or 237, wherein the subject experiences a decrease in the MG-ADL score by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.
239. A method for treating myasthenia gravis in a subject that result in an increase in muscle strength, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
240. The method according to item 239, wherein the improvement in muscle strength is determined by measuring grip strength using a handheld dynamometer.
241. The method according to one of items 239 or 240, wherein the therapeutically effective dose provides a Cₘₐₓ in the range of 2,790 ng/mL to 76,700 ng/mL in the subject.
242. The method according to one of items 239 to 241, wherein the muscle strength has increased by at least 5%, such as at least 10%, such as at least 15%, such as at least 20%, such as at least 25%, such as at least 30%, such as at least 50%, such as at least 75%, such as at least 100%, such as at least 150%, such as at least 200% such as between 10% and 400%, such as between 15% and 200%, such as between 20% and 100%.
243. The method according to one of items 239 to 241, wherein the grip strength has increased by at least 0.25 kg, such as at least 0.50 kg, such as at least 0.75 kg, such as at least 1.0 kg, such as at least 1.25 kg, such as at least 1.5 kg, such as at least 1.75 kg, such as at least 2.0 kg, such as at least 2.5 kg, such as at least 3.0 kg, such as between 0.25 and 5.0 kg, such as between 0.25 and 4.0 kg, such as between 0.5 and 4.0 kg.
244. A method for treating myasthenia gravis in a subject that result in a decrease in the Myasthenia Gravis Composite (MGC) scale, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
245. The method according to item 244, wherein the therapeutically effective dose provides a Cₘₐₓ in the range of 2,790 ng/mL to 76,700 ng/mL in the subject.
246. The method according to one of items 244 or 245, wherein the subject experiences a decrease in the MGC scale by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.
247. A method for treating myasthenia gravis in a subject that result in a decrease in the Myasthenia Gravis Quality of Life 15 (MG-QOL15) score, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
248. The method according to item 247, wherein the therapeutically effective dose provides a Cₘₐₓ in the range of 2,790 ng/mL to 76,700 ng/mL in the subject.
249. The method according to one of items 247 or 248, wherein the subject experiences a decrease in the MG-QOL15 score by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.
250. A method for treating myasthenia gravis in a subject that result in an improvement in health state when determined using the EQ-5D scale, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
251. The method according to item 250, wherein the therapeutically effective dose provides a Cₘₐₓ in the range of 2,790 ng/mL to 76,700 ng/mL in the subject.
252. The method according to one of items 250 or 251, wherein the subject experiences an increase in the EQ-5D scale by at least 0.3 points, such as at least 0.5 points, such as at least 0.75 points, such as at least 1 point, such as at least 2 points, such as at least 3 points, such as at least 4 points, such as at least 5 points, such as at least 6 points, such as at least 8 points, such as at least 10 points, such as between 0.3 and 10 points, such as between 0.5 and 6 points, such as between 0.3 and 5 points.
253. A method for treating myasthenia gravis in a subject that result in a decrease in a reduction in jitter, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
254. The method according to item 253, wherein the reduction in jitter is determined using single fibre electromyography.
255. The method according to one of items 253 or 254, wherein the therapeutically effective dose provides a Cₘₐₓ in the range of 2,790 ng/mL to 76,700 ng/mL in the subject.
256. The method according to one of items 253 to 255, wherein the jitter has been reduced by at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%,
257. The method according to one of items 253 to 256, wherein the jitter has been reduced by at least 5 μs, such as at least 10 μs, such as at least 15 μs, such as at least 20 μs, such as at least 25 μs, such as at least 30 μs, such as at least 40 μs, such as at least 50 μs, such as at least 75 μs, such as at least 100 μs, such as between 5 μs and 200 μs, such as between 5 μs and 100 μs, such as between 10 μs and 50 μs.
258. A method for treating myasthenia gravis in a subject that result in a reduction in blocking, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
259. The method according to item 258, wherein the reduction in blocking is determined using single fibre electromyography.
260. The method according to one of items 258 or 259, wherein the therapeutically effective dose provides a Cₘₐₓ in the range of 2,790 ng/mL to 76,700 ng/mL in the subject.
261. The method according to one of items 258 to 260, wherein the blocking has been reduced by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
262. A method for treating myasthenia gravis in a subject that result in a decrease in the Individualised Neuromuscular Quality of Life score, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
263. The method according to item 262, wherein the therapeutically effective dose provides a Cₘₐₓ in the range of 2,790 ng/mL to 76,700 ng/mL in the subject.
264. The method according to one of items 262 or 263, wherein the Individualised Neuromuscular Quality of Life score is decreased by at least 0.5 points, such as at least 1 point, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
265. A method for treating myasthenia gravis in a subject that result in a decrease in the Fatigue Severity Scale score, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
266. The method according to item 265, wherein the therapeutically effective dose provides a Cₘₐₓ in the range of 2,790 ng/mL to 76,700 ng/mL in the subject.
267. The method according to one of items 265 or 266, wherein the Fatigue Severity Scale score is decreased by at least 0.5 points, such as at least 0.75 points, such as at least 1 point, such as at least 1.5 points, such as at least 2 points, such as at least 3 points, such as at least 4 points, such as at least 5 points, such as at least 6 points, such as at least 8 points, such as at least 10 points, such as at least 15 points, such as at least 20 points, such as between 0.5 and 30 points, such as between 1 and 20 points, such as between 0.5 and 10 points.
268. A method for treating myasthenia gravis in a subject that result an improvement in pulmonary function, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
269. The method according to item 268, wherein the improvement in pulmonary function is determined by measuring forced vital capacity (FVC), forced expiratory volume in 1 second (FEV1), maximal inspiratory pressure (MIP), and/or maximal expiratory pressure (MEP).
270. The method according to one of items 268 or 269, wherein the therapeutically effective dose provides a Cₘₐₓ in the range of 2,790 ng/mL to 76,700 ng/mL in the subject.
271. The method according to one of items 268 to 270, wherein the subject experiences an improvement in pulmonary function determined by measuring forced vital capacity (FVC), wherein FVC has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
272. The method according to one of items 268 to 270, wherein the subject experiences an improvement in pulmonary function determined by measuring forced expiratory volume in 1 second (FEV1), wherein FEV1 has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
273. The method according to one of items 268 to 270, wherein the subject experiences an improvement in pulmonary function determined by measuring maximal inspiratory pressure (MIP), wherein MIP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
274. The method according to one of items 268 to 270, wherein the subject experiences an improvement in pulmonary function determined by measuring maximal expiratory pressure (MEP), wherein MEP has increased by at least 5%, such as at least 10%, such as at least 15%, such as at least 20%, such as at least 25%, such as at least 30%, such as at least 50%, such as at least 75%, such as at least 100%, such as at least 150%, such as at least 200%, such as between 5% and 95%, such as between 5% and 80%, such as between 10% and 50%.
275. A method for treating myasthenia gravis in a subject that result in a decrease in the Quantitative Myasthenia Gravis (QMG) total score, comprising administering a therapeutically effective dose of (2S)-2-[4-bromo-2-(1,2-oxazol-3-yl)phenoxy]propanoic acid, or a pharmaceutically acceptable salt, hydrate, polymorph, tautomer, or solvate thereof, to the subject, wherein the therapeutic dose is within the range of 100 mg to 1500 mg.
276. The method according to item 275, wherein the therapeutically effective dose provides a Cₘₐₓ in the range of 2,790 ng/mL to 76,700 ng/mL in the subject.
277. The method according to one of items 275 to 276, wherein the subject experiences a decrease in the QMG total score by at least 0.9 points, such as at least 1.0 point, such as at least 1.5 points, such as at least 2.0 points, such as at least 3.0 points.
278. A method for improving the Quantitative Myasthenia Gravis total score in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.
279. The method according to item 278, wherein the reduction in QMG total score after treatment is at least 0.9 points, such as at least 1.0 point, such as at least 1.5 points, such as at least 2.0 points, such as at least 3.0 points.
280. The method according to item 278, wherein the reduction in QMG total score after treatment is at least 0.9 points, such as at least 1.0 point, such as at least 1.5 points, such as at least 2.0 points, such as at least 3.0 points compared to placebo at the same time point.
281. The method according to item 278, wherein the reduction in QMG total score after treatment is between 2 and 5 hours, such as 2 hours after treatment, such as 3 hours after treatment, such as 4 hours after treatment or such as 5 hours after treatment.
282. A method of improving right hand grip strength in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.
283. The method according to item 282, wherein the improvement in right hand grip strength is at least 2.0 kg, such as at least 2.5 kg.
284. The method according to item 282, wherein the improvement in right hand grip strength is at least 2.0 kg, such as at least 2.5 kg compared to placebo at the same time point.
285. A method of reducing decrement in EMG in repetitive nerve stimulation in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.
286. A method of improving the symptoms of double vision in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.
287. A method of improving the symptoms of ptosis in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.
288. A method of improving the symptoms of dysarthria in a subject suffering from myasthenia gravis, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.
289. A method for enhancing neuromuscular transmission and/or restoration of skeletal muscle function, comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, to the subject.
290. A method for treatment of myasthenia gravis comprising administering (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor to a subject in need thereof.
291. A method of treating myasthenia gravis that results in an improvement of a quantitative myasthenia gravis total score in a patient in need thereof, the method comprising administering a therapeutically effective dose of a compound of (2S)-2-[4-bromo-2-(1,2-oxazol-3-yl)phenoxy]propanoic acid or a pharmaceutically acceptable salt, hydrate, polymorph, tautomer, or solvate thereof,
   wherein the therapeutically effective dose is within the range of 100 mg to 1500 mg and provides a Cₘₐₓ in the range of 5,000 ng/mL to 14,000 ng/mL in the patient,
   wherein after administration of the therapeutically effective dose of the compound, the patient experiences at least a 0.9 point reduction in their quantitative myasthenia gravis score when compared to placebo at the same time point.
292. The method of item 291, wherein the therapeutically effective dose of the compound further provides an AUC_{inf} in the range of 15,000 ng/mL to 500,000 ng/mL in the patient.
293. The method of item 291, wherein the therapeutically effective dose of the compound has a Tₘₐₓ in the patient ranging from 1 to 5 hours.
294. The method of item 291, wherein the therapeutically effective dose of the compound has a half-life in the patient ranging from 3 hours to 7 hours.
295. The method of item 291, wherein the therapeutically effective dose of the compound is administered orally to the patient.
296. The method of item 291, wherein the patient experiences at least a 1.0 point reduction, such as at least 1.5 point reduction, such as at least 2.0 point reduction in their quantitative myasthenia gravis score.
297. The method of item 291, wherein the Myasthenia Gravis Foundation of America (MGFA) clinical classification of the patient is class I, IIa, IIb IIIa, IIIb, IVa, IVb or V prior to treatment.
298. A method of treating myasthenia gravis that results in an improvement of right-hand grip strength in a patient in need thereof, the method comprising administering a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof,
   wherein the therapeutically effective dose is within the range of 100 mg to 1500 mg and provides a Cₘₐₓ in the range of 5,000 ng/mL to 14,000 ng/mL in the patient,
   wherein after administration of the therapeutically effective dose of the compound, the patient experiences at least a 1 kg improvement in their hand grip strength when compared to their hand grip strength before administration.
299. The method of item 298, wherein the at least 1 kg improvement in the patient's hand grip strength occurs at least 3 hours after administration of the therapeutically effective dose of the compound
300. The method of item 298, wherein the therapeutically effective dose of the compound further provides an AUC_{inf} in the range of 15,000 h·ng/mL to 500,000 h·ng/mL in the patient.
301. The method of item 298, wherein the therapeutically effective dose of the compound has a Tₘₐₓ in the patient ranging from 1 to 5 hours.
302. The method of item 298, the therapeutically effective dose of the compound has a half-life in the patient ranging from 3 hours to 7 hours.
303. The method of item 298, wherein the therapeutically effective dose of the compound is administered orally to the patient.
304. The method of item 298, wherein the therapeutically effective dose is within the range of 100 mgs to 600 mgs.
305. The method of item 298, wherein the therapeutically effective dose is within the range of 200 mgs to 600 mgs.
306. The method of item 298, wherein the therapeutically effective dose is 400 mgs.
307. The method of item 298, wherein the therapeutically effective dose is 350 mgs.
308. The method of item 298, wherein the therapeutically effective dose is 300 mgs.
309. The method of item 298, wherein the therapeutically effective dose is 250 mgs.
310. The method of item 298, wherein the therapeutically effective dose is 200 mgs.
311. The method of item 298, wherein the therapeutically effective dose is 150 mgs.
312. The method of item 298, wherein the therapeutically effective dose is 100 mgs.
313. The method of item 298, wherein the therapeutically effective dose is administered once, twice or three times daily.
314. A method for treating a patient suffering from symptoms of myasthenia gravis, the method comprising administering a therapeutically effective dose of (2S)-2-[4-bromo-2-(1,2-oxazol-3-yl)phenoxy]propanoic acid or a pharmaceutically acceptable salt, hydrate, polymorph, tautomer, or solvate thereof, wherein the therapeutically effective dose is within the range of 100 mgs to 1500 mgs.
315. The method of item 314, wherein the therapeutically effective dose is within the range of 100 mgs to 600 mgs.
316. The method of item 314, wherein the therapeutically effective dose is within the range of 200 mgs to 600 mgs.
317. The method of item 314, wherein the therapeutically effective dose is 100 mgs.
318. The method of item 314, wherein the therapeutically effective dose is 150 mgs.
319. The method of item 314, wherein the therapeutically effective dose is 200 mgs.
320. The method of item 314, wherein the therapeutically effective dose is 250 mgs.
321. The method of item 314, wherein the therapeutically effective dose is 300 mgs.
322. The method of item 314, wherein the therapeutically effective dose is 350 mgs.
323. The method of item 314, wherein the therapeutically effective dose is 400 mgs.
324. The method of item 314, wherein the therapeutically effective dose is 500 mgs.
325. The method of item 314, wherein the therapeutically effective dose is 600 mgs.
326. The method of item 314, wherein the therapeutically effective dose is administered once, twice, three or four times daily.
327. The method according to any of items 234 to 314, wherein the composition is administered at a therapeutic dose of 100 to 1500 mg of (2S)-2-[4-bromo-2-(1,2-oxazol-3-yl)phenoxy]propanoic acid.
328. The method according to any of items 234 to 314, wherein the composition is administered at a therapeutic dose of 100 to 600 mg of (2S)-2-[4-bromo-2-(1.2-oxazol-3-yl)phenoxy]propanoic acid and the composition is to be administered one time daily.
329. The method according to any of items 234 to 314, wherein the composition is administered at a therapeutic dose of 200 to 600 mg of (2S)-2-[4-bromo-2-(1,2-oxazol-3-yl)phenoxy]propanoic acid and the composition is to be administered one time daily.
330. The method according to any of items 234 to 314, wherein the composition is administered at a therapeutic dose of 100 to 600 mg of (2S)-2-[4-bromo-2-(1,2-oxazol-3-yl)phenoxy]propanoic acid and the composition is to be administered two times daily.
331. The method according to any of items 234 to 314, wherein the composition is administered at a therapeutic dose of 200 to 600 mg of (2S)-2-[4-bromo-2-(1,2-oxazol-3-yl)phenoxy]propanoic acid and the composition is to be administered two times daily.
332. The method according to any of items 234 to 314, wherein the composition is administered at a therapeutic dose of 100 to 600 mg of (2S)-2-[4-bromo-2-(1,2-oxazol-3-yl)phenoxy]propanoic acid and the composition is to be administered three times daily.
333. The method according to any of items 234 to 314, wherein the composition is administered at a therapeutic dose of 200 to 600 mg of (2S)-2-[4-bromo-2-(1,2-oxazol-3-yl)phenoxy]propanoic acid and the composition is to be administered three times daily.
334. The method according to one of items 234 to 314, wherein the composition is administered at a therapeutic dose of 100 to 600 mg of (2S)-2-[4-bromo-2-(1,2-oxazol-3-yl)phenoxy]propanoic acid and the composition is to be administered four times daily.
335. The method according to one of items 234 to 314, wherein the composition is administered at a therapeutic dose of 200 to 600 mg of (2S)-2-[4-bromo-2-(1,2-oxazol-3-yl)phenoxy]propanoic acid and the composition is to be administered four times daily.
336. The method according to one of items 234 to 314, wherein the composition is administered at a therapeutic dose of 100 mg of (2S)-2-[4-bromo-2-(1,2-oxazol-3-yl)phenoxy]propanoic acid and the composition is to be administered one time daily.
337. The method according to one of items 234 to 314, wherein the composition is administered at a therapeutic dose of 100 mg of (2S)-2-[4-bromo-2-(1,2-oxazol-3-yl)phenoxy]propanoic acid and the composition is to be administered two times daily.
338. The method according to one of items 234 to 314, wherein the composition is administered at a therapeutic dose of 100 mg of (2S)-2-[4-bromo-2-(1,2-oxazol-3-yl)phenoxy]propanoic acid and the composition is to be administered three times daily.
339. The method according to one of items 234 to 314, wherein the composition is administered at a therapeutic dose of 100 mg of (2S)-2-[4-bromo-2-(1,2-oxazol-3-yl)phenoxy]propanoic acid and the composition is to be administered four times daily.
340. The method according to one of items 234 to 314, wherein the composition is administered at a therapeutic dose of 150 mg of (2S)-2-[4-bromo-2-(1,2-oxazol-3-yl)phenoxy]propanoic acid and the composition is to be administered one time daily.
341. The method according to one of items 234 to 314, wherein the composition is administered at a therapeutic dose of 150 mg of (2S)-2-[4-bromo-2-(1,2-oxazol-3-yl)phenoxy]propanoic acid and the composition is to be administered two times daily.
342. The method according to one of items 234 to 314, wherein the composition is administered at a therapeutic dose of 150 mg of (2S)-2-[4-bromo-2-(1,2-oxazol-3-yl)phenoxy]propanoic acid and the composition is to be administered three times daily.
343. The method according to one of items 234 to 314, wherein the composition is administered at a therapeutic dose of 150 mg of (2S)-2-[4-bromo-2-(1,2-oxazol-3-yl)phenoxy]propanoic acid and the composition is to be administered four times daily.
344. The method according to one of items 234 to 314, wherein the composition is administered at a therapeutic dose of 200 mg of (2S)-2-[4-bromo-2-(1,2-oxazol-3-yl)phenoxy]propanoic acid and the composition is to be administered one time daily.
345. The method according to one of items 234 to 314, wherein the composition is administered at a therapeutic dose of 200 mg of (2S)-2-[4-bromo-2-(1,2-oxazol-3-yl)phenoxy]propanoic acid and the composition is to be administered two times daily.
346. The method according to one of items 234 to 314, wherein the composition is administered at a therapeutic dose of 200 mg of (2S)-2-[4-bromo-2-(1,2-oxazol-3-yl)phenoxy]propanoic acid and the composition is to be administered three times daily.
347. The method according to one of items 234 to 314, wherein the composition is administered at a therapeutic dose of 200 mg of (2S)-2-[4-bromo-2-(1,2-oxazol-3-yl)phenoxy]propanoic acid and the composition is to be administered four times daily.
348. The method according to one of items 234 to 314, wherein the composition is administered at a therapeutic dose of 250 mg of (2S)-2-[4-bromo-2-(1,2-oxazol-3-yl)phenoxy]propanoic acid and the composition is to be administered one time daily.
349. The method according to one of items 234 to 314, wherein the composition is administered at a therapeutic dose of 250 mg of (2S)-2-[4-bromo-2-(1,2-oxazol-3-yl)phenoxy]propanoic acid and the composition is to be administered two times daily.
350. The method according to one of items 234 to 314, wherein the composition is administered at a therapeutic dose of 250 mg of (2S)-2-[4-bromo-2-(1,2-oxazol-3-yl)phenoxy]propanoic acid and the composition is to be administered three times daily.
351. The method according to one of items 234 to 314, wherein the composition is administered at a therapeutic dose of 250 mg of (2S)-2-[4-bromo-2-(1,2-oxazol-3-yl)phenoxy]propanoic acid and the composition is to be administered four times daily.
352. The method according to any of items 234 to 314, wherein the composition is administered at a therapeutic dose of about 300 mg of (2S)-2-[4-bromo-2-(1,2-oxazol-3-yl)phenoxy]propanoic acid and the composition is to be administered one time daily.
353. The method according to any of items 234 to 314, wherein the composition is administered at a therapeutic dose of about 300 mg of (2S)-2-[4-bromo-2-(1,2-oxazol-3-yl)phenoxy]propanoic acid and the composition is to be administered two times daily.
354. The method according to any of items 234 to 314, wherein the composition is administered at a therapeutic dose of about 300 mg of (2S)-2-[4-bromo-2-(1,2-oxazol-3-yl)phenoxy]propanoic acid and the composition is to be administered three times daily.
355. The method according to one of items 234 to 314, wherein the composition is administered at a therapeutic dose of 300 mg of (2S)-2-[4-bromo-2-(1,2-oxazol-3-yl)phenoxy]propanoic acid and the composition is to be administered four times daily.
356. The method according to one of items 234 to 314, wherein the composition is administered at a therapeutic dose of 350 mg of (2S)-2-[4-bromo-2-(1,2-oxazol-3-yl)phenoxy]propanoic acid and the composition is to be administered one time daily.
357. The method according to one of items 234 to 314, wherein the composition is administered at a therapeutic dose of 350 mg of (2S)-2-[4-bromo-2-(1,2-oxazol-3-yl)phenoxy]propanoic acid and the composition is to be administered two times daily.
358. The method according to one of items 234 to 314, wherein the composition is administered at a therapeutic dose of 350 mg of (2S)-2-[4-bromo-2-(1,2-oxazol-3-yl)phenoxy]propanoic acid and the composition is to be administered three times daily.
359. The method according to one of items 234 to 314, wherein the composition is administered at a therapeutic dose of 350 mg of (2S)-2-[4-bromo-2-(1,2-oxazol-3-yl)phenoxy]propanoic acid and the composition is to be administered four times daily.
360. The method according to one of items 234 to 314, wherein the composition is administered at a therapeutic dose of 400 mg of (2S)-2-[4-bromo-2-(1,2-oxazol-3-yl)phenoxy]propanoic acid and the composition is to be administered one time daily.
361. The method according to any of items 234 to 314, the composition is administered at a therapeutic dose of about 400 mg of (2S)-2-[4-bromo-2-(1,2-oxazol-3-yl)phenoxy]propanoic acid and the composition is to be administered two times daily.
362. The method according to any of items 234 to 314, wherein the composition is administered at a therapeutic dose of about 400 mg of (2S)-2-[4-bromo-2-(1,2-oxazol-3-yl)phenoxy]propanoic acid and the composition is to be administered three times daily.
363. The method according to any of items 234 to 314, wherein the composition is administered at a therapeutic dose of about 400 mg of (2S)-2-[4-bromo-2-(1,2-oxazol-3-yl)phenoxy]propanoic acid and the composition is to be administered four times daily.
364. The method according to any of items 234 to 314, wherein the composition is administered at a therapeutic dose of about 500 mg of (2S)-2-[4-bromo-2-(1,2-oxazol-3-yl)phenoxy]propanoic acid and the composition is to be administered two times daily.
365. The method according to any of items 234 to 314, wherein the composition is administered at a therapeutic dose of about 500 mg of (2S)-2-[4-bromo-2-(1,2-oxazol-3-yl)phenoxy]propanoic acid and the composition is to be administered three times daily.
366. The method according to any of items 234 to 314, wherein the composition is administered at a therapeutic dose of about 600 mg of (2S)-2-[4-bromo-2-(1,2-oxazol-3-yl)phenoxy]propanoic acid and the composition is to be administered two times daily.
367. The method according to any of items 234 to 314, wherein the composition is administered at a therapeutic dose of about 600 mg of (2S)-2-[4-bromo-2-(1,2-oxazol-3-yl)phenoxy]propanoic acid and the composition is to be administered three times daily.
368. Use of a composition comprising (2S)-2-[4-bromo-2-(1,2-oxazol-3-yl)phenoxy]propanoic acid, or a pharmaceutically acceptable salt, hydrate, polymorph, tautomer, or solvate thereof, in the manufacture of a medicament for treatment of myasthenia gravis in a subject, wherein the composition is for administration at a therapeutic dose of 100 to 1500 mg of (2S)-2-[4-bromo-2-(1,2-oxazol-3-yl)phenoxy]propanoic acid.
369. A kit-of-parts or a composition comprising (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor for use in a method for treatment of myasthenia gravis in a subject.
370. The kit-of-parts or composition for use according to item 369, wherein (2S)-2-[4-bromo-2-(1,2-oxazol-3-yl)phenoxy]propanoic acid, or a pharmaceutically acceptable salt, hydrate, polymorph, tautomer, or solvate thereof, is for administration at a therapeutic dose of 100 to 1500 mg of (2S)-2-[4-bromo-2-(1,2-oxazol-3-yl)phenoxy]propanoic acid.
371. The kit-of-parts or composition for use according to any one of items 369 to 370, wherein the therapeutic dose is as defined in any one of items 2 to 57.
372. The kit-of-parts or composition for use according to any one of items 369 to 371, wherein the (2S)-2-[4-bromo-2-(1,2-oxazol-3-yl)phenoxy]propanoic acid, or a pharmaceutically acceptable salt, hydrate, polymorph, tautomer, or solvate thereof, and the acetylcholinesterase inhibitor are administered simultaneously.
373. The kit-of-parts or composition for use according to any one of items 369 to 371, wherein the (2S)-2-[4-bromo-2-(1,2-oxazol-3-yl)phenoxy]propanoic acid, or a pharmaceutically acceptable salt, hydrate, polymorph, tautomer, or solvate thereof, and the acetylcholinesterase inhibitor are administered sequentially.
374. The kit-of-parts or composition for use according to any one of items 369 to 373, wherein the acetylcholinesterase inhibitor is pyridostigmine.
375. Use of a kit-of-parts or a composition comprising (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor for the manufacture of a medicament for the treatment of myasthenia gravis.
376. The composition for use, the method or the combination for use according to any one of the preceding items, wherein the subject is a human being.
377. The composition for use, the method or the combination for use according to any one of the preceding items, wherein the subject is suffering from myasthenia gravis.
378. The composition for use, the method or the combination for use according to any one of the preceding items, wherein the subject is suffering from ocular myasthenia gravis, early-onset generalised myasthenia gravis, late-onset generalised myasthenia gravis, generalized myasthenia gravis, seropositive myasthenia gravis, seronegative myasthenia gravis, AChR antibody positive myasthenia gravis, or muscle-specific kinase antibody-positive myasthenia gravis (MuSK-MG).
379. The composition for use, the method or the combination for use according to any one of the preceding items, wherein the subject is also administered one of more compounds selected from the list consisting of amifampridine, Eculizumab, Prednisolone, Prednisone, Azathioprine, Soliris, Rituximab, Efgartigimod alfa, Zilucoplan, Rozanolixizumab, Cholecalciferol and immunoglobulin.

### EXAMPLES

### Example 1: EAMG Model

All handling, use, and housing of animals complied with European and Danish Animal Welfare regulations, including euthanasia. To test for efficacy of CIC-1 inhibition in conditions of compromised neuromuscular transmission, experimental autoimmune MG (EAMG) in rats was chosen as a model for neuromuscular transmission disorders being a recognized model for determination of the efficacy of medical treatment of MG (Losen, et al., Exp. Neurol. 2015, 270:18-28). All activities pertaining to handling, disease induction and testing of novel compounds were covered by licences numbered 2018-15-0201-01408 and 2018-15-0201-01420.

To induce EAMG, 250 μL of emulsion of Complete Freund's Adjuvant, consisting of heat-inactivated Mycobacterium tuberculosis H37Ra in non-metabolizable oils, together with Phosphate-Buffered Saline (PBS) and 80 μg of the extracellular α-domain peptide fragment of the human nicotinic acetylcholine receptor (antigen) was injected subcutaneously in seven-week-old, anaesthetized Lewis rats. The disease-inducing antigen was supplied by the Hellenic Pasteur institute3. The emulsion was deposited at the tail base, left hindleg, and right foreleg. After injection, animals were allowed to recover and returned to their home cage maintained in ventilated racks under controlled temperature (20 to 22° C.) and humidity (˜55%) control, under a 12-h light/12-h dark cycle with food and water provided ad libitum. The animals had pathogen free status and the housing and changing system was designed to assure that the pathogen free status was preserved during the study. The EAMG rats were observed, weighed, and scored for MG symptoms weekly until symptoms of MG developed, whereafter animals were observed daily.

The scoring of MG symptoms consisted of a visual inspection of the animal as described elsewhere (Losen et al., Exp. Neurol. 2015, 270:18-28). If there were obvious signs of fatigue, the animal was not examined further. However, if no clear sign of fatigue could be observed, a forced exercise was conducted in which the animal pulled a grid on a device to measure grip force for 30 seconds, at least 10 times. This was done to unmask any muscle fatigue. After the exercise, the animal was observed again and if clinical signs of fatigue had developed, the animal was scored 1, according to the scoring system below:
0; No clinical signs observed.
1; No clinical signs observed before fatiguing exercise, but symptoms of fatigue/weakening present after the exercise.
2; Clinical symptoms observed before fatiguing exercise. Symptoms could be hunched back, resting of head, fatigue, and/or shaking.
3; Paralysis of hindlegs, struggled breathing, hindered movement, lack of ability to grip, and/or distinct fatigue (Humane Endpoint).
4; Moribund (Humane Endpoint).

Disease onset was expected five to seven weeks after immunization. When an animal displayed symptoms equal to EAMG score 1, the weighing and frequency of disease scoring was increased to 3 times per week, and standard feeding was supplemented with softened breeding feed and/or diet-gel to mitigate possible accelerated weight loss. When an animal displayed symptoms equal to EAMG score 2, the animal was weighed and scored once per day, and at least inspected morning and afternoon each day. Animals with small wounds at the site for injection of the immunization emulsion were treated with antibacterial ointment, until the wounds healed. An animal showing clinical signs of moderate pain, moderate distress, or any degree of suffering was handled as appropriate, this being discontinuing the administration or euthanizing the animal.

No animals had to be discontinued treatment with either of the CIC-1 inhibitors. Limits for disease severity requiring that an animal was humanely euthanized followed the European and Danish legislation on animals in experimentation. Specifically, this included animals that displayed disease progression to EAMG score of 3 or had a body weight loss of more than 20% as compared to the individual maximum body weight prior to disease induction. Educated personnel under veterinary supervision handled the animals. Health monitoring of the animal facilities was conducted according to standard operation procedures. Daily records and decisions were made concerning animal welfare.

### Experiments Involving Isolated Nerve-Muscle Preparations Form Healthy Rats and EAMG Rats

To allow for measurements of electrophysiological properties at the cellular level and for measurements of force, it was necessary to isolate nerve-muscle preparations from the rats. To this end, animals were sacrificed, and muscles were dissected out with 1 to 3 cm of intact nerve. The nerve-muscle preparations were then transferred to the organ baths used for the particular experiments (see below) and perfused with Normal Krebs-Ringer (NKR) solution, consisting of (in mM); 122 NaCl, 25 NaHCO₃, 2.8 KCl, 1.2 KH₂PO₄, 1.2 MgSO₄ 1.27 CaCl₂ and 5 D-glucose. This NKR solution was continuously gassed with a mixture of 95% oxygen and 5% CO₂, to maintain a pH of approx. 7.4. In some experiments methyl-sulphate was used to replace Cl⁻ in the solution. All chemicals used were of analytical grade.

### Example 2: Membrane Conductance and Rheobase in Muscle Fibres of Isolated Nerve-Muscle Preparations

The capacity of compounds to inhibit CIC-1 in native tissue could be evaluated by measuring their effect on the resting membrane conductance (Gₘ) in single muscle fibres of freshly dissected intact muscles from adult rats. Gₘ is an electrical measure of the flow of ions across the surface membrane through the ion channels that are open at the resting membrane potential. CIC-1 is known to be responsible for around 80% of Gₘ in both rat and man, and it is the only known surface membrane Cl⁻ ion channel in skeletal muscle (Pedersen et al., J. Gen. Physiol. 2016, 147:291-308). Any changes in Gₘ with a compound can therefore largely be taken to reflect alterations of CIC-1 function. Nevertheless, an effect of a compound on CIC-1 can be isolated by comparing recordings of Gₘ between experiments with and without Cl⁻ in the experimental solution.

Experimentally, soleus or diaphragm muscles from healthy rats and EAMG rats with score 0, 1 or 2 were used. After dissection, the muscles were mounted in an organ bath chamber perfused with NKR solution at a temperature of 30-31° C., pH 7.4-7.5. To allow selection of fibres to measure from, the chamber was placed on the XY-table of a Nikon upright microscope (Eclipse FN1, DFA, Glostrup, Denmark) enabling movement of the chamber and visual inspection of fibres and electrodes. Muscles were allowed to rest for 30 mins in the solution before beginning the experiment.

The resting Gₘ was measured using electrophysiological technique that involves insertion of 3 intracellular microelectrodes into the individual muscle fibre as explained in detail elsewhere (Riisager, A., Aarhus University, 2015). Briefly, the 3 electrodes (E1-E3) were placed into the same muscle fibre such that three different inter-electrode distances could be identified. (E1→E2=X1; E2→E3=X2; E1→E3=X3) with X2 being roughly twice the distance of X1. All electrodes recorded the membrane potential, and two electrodes were used to inject a current protocol: A 50 ms steady state current (I) of −30-50 nA was first injected by E1 while the steady membrane potential responses (ΔV) were measured with E2 and E3 yielding recordings at X1 and X3, respectively.

Subsequently, same duration and size current was injected by E3 while AV were measured with E1 and E2 yielding another recording at X3 and, a recording at X2. Collectively, this resulted in the measurement of ΔV at three different distances on the fibre (X1, X2, X3) with X3 being recorded twice by both E1 and E3. From these recordings, the transfer resistance could be calculated (ΔV/1) at the three distances and Gₘ determined using linear cable theory as previously described.

Once Gₘ had been determined in a fibre, a series of 25 ms positive currents of increasing amplitude from 5 to 70 nA in 5 nA increments were injected through E1. This was done to determine the current required for an AP to be elicited in the impaled fibre. This current is known as the rheobase current and it is a measure of the fibre excitability. It was included to explore the hypothesis that CIC-1 inhibition would increase muscle fibre excitability reflected by a reduction in rheobase current, after addition of a CIC-1 inhibitor compound.

To test the effect of a compound on Gₘ and rheobase current, recordings were first obtained from a range of muscle fibres before adding compound and then the compound was added at variable concentrations. The muscle preparations were incubated for 20-30 min at each compound concentration before initiating measurements and recordings were obtained from approx. 10 fibres per concentration per muscle. The affinity of NMD670 for inhibiting CIC-1 was determined from plots of Gₘ before compound and at the different compound concentrations to which a four parameter Hill function with variable slope was fitted to extract an apparent affinity

To demonstrate the effect of NMD670 on CIC-1 inhibition and excitability in muscle fibres from EAMG animals, Gₘ and rheobase currents were determined in fibres from intact EAMG soleus muscles before and after adding 20 μM of NMD670 to the experimental solution (FIG. 1). The effect of NMD670 on membrane conductance (Gₘ) in rat soleus muscle from healthy (n=20) and EAMG animals (n=12) as the average Gₘ measured in a muscle before (black circles or open squares respectively) and after addition of 20 μM NMD670 (black triangles or open inverted triangles respectively) is shown in FIG. 1. Addition of NMD670 statistically significantly reduced Gₘ in both healthy and EAMG animals.

The effect of NMD670 on skeletal muscle fibre excitability as evaluated from rheobase current in rat soleus muscle from healthy (n=20) and EAMG animals (n=12) before (black circles or open squares respectively) and after addition of 20 μM NMD670 (black triangles or open inverted triangles respectively) is shown in FIG. 2. Addition of NMD670 significantly reduced rheobase current in muscle fibres from both groups of rats.

To confirm that NMD670 exerted its reduction on Gₘ through CIC-1 channel inhibition, experiments with bathing solution free of Cl⁻ were conducted with increasing concentration of NMD670 and under these conditions NMD670 did not reduced Gₘ.

### Example 3: Endplate Potentials

The effect of CIC-1 inhibition on neuromuscular transmission was investigated at the cellular level by recording endplate potentials (EPPs) with intracellular electrodes in isolated nerve-muscle preparations from healthy and EAMG rats (score 0-2). Muscle preparations used in the experiments were diaphragm or levator longus auris (LAL) providing the advantage to dissect out 1-3 cm of intact motor nerve. After dissection, preparations were immediately mounted in a chamber perfused with NKR at a temperature of 30-31° C., pH 7.4-7.5. The preparations were allowed to rest 30 min in the solution before beginning the experiment.

To establish stable, reliable stimulation of the motor nerve, a field stimulation electrode was developed to selectively stimulate the motor nerve. The electrode consisted of two silver wires isolated except at their tip and the electrode was positioned with the nerve in between the two un-isolated ends of the wires. The wires were attached to a glass pipette in a pipette holder that was positioned with a micromanipulator placed on the XY table of the microscope. The electrode allowed continuous stimulation of the nerve while avoiding degradation observed when using a suction electrode (data not shown). To avoid direct muscle fibre stimulation and to minimize stimulation artefacts in the recordings, the electrode was placed as far from the muscle itself as possible. Trigger pulses for nerve stimulations were delivered by an external constant current stimulator (DS3 Isolated Constant Current Stimulator; Digitimer, U.S.) controlled by the recording software (Signal version 6.4, Cambridge Electronics Design Ltd, Cambridge, UK). Before initiation of the experimental protocol, the external stimulator was adjusted to deliver the minimum current size and duration required to evoke an EPP in the muscle.

This was done to avoid nerve damage and, again, to minimize the risk of direct stimulation of the muscle through the bath instead of through the nerve.

To record EPPs evoked by nerve stimulation, a glass microelectrode was inserted into individual muscle fibres in close vicinity to the neuromuscular junctions. The relatively thin layers of muscle fibres and transparent characteristics of diaphragm and LAL made it possible to locate endplates by eye through the microscope. To further verify the position of the microelectrode at the endplate, recordings were only included if observations of miniature endplate potentials were made when the electrode was first inserted. Action potential excitation in response to nerve stimulation was avoided using 0.5-1 μM of the voltage gated Na⁺-channel blocker μ-conotoxin GIIIB (Alamone Labs, IL).

The stimulation protocol for evoking EPPs consisted of 2 different trains of trigger pulses, first at 12 Hz for 30 pulses, and then a second train at 30 Hz for 30 pulses. There was 10 s of rest between the two trains. This resulted in recordings of 2 EPP trains from each fibre which EPP amplitudes were analyzed. For each muscle, a set of control measurements was first obtained from approx. 20 fibres before compound, and then from approx. 20 fibres starting after 20-30 mins of incubation at 20 μM NMD670.

To account for slight variation in resting membrane potentials between fibres, the EPP amplitudes were corrected using the following expression:

$$EP {P}_{AMP , Corr} = EP {P}_{A M P} \frac{{V}_{m , 0} - {E}_{R e ν}}{{V}_{m} - {E}_{R e ν}}$$

Where EPPₐₘₚ and EPP_{amp,corr} refer to EPP amplitudes before and after correction, respectively. V_{m,0} is the membrane potential that all fibres were corrected to (set to −80 mV) and Vₘ is the membrane potential actually recorded. E_{Rev} is the reversal potential for the acetylcholine receptor and is set to −15 mV (del Castillo et al, J. Physiol. 1954, 124:560-573).

FIG. 3 shows that EPP were markedly reduced in muscle fibres from EAMG rats versus healthy animals, but that EPP in muscle fibres from EAMG rats was restored after addition of NMD670. FIG. 4 shows the average endplate potential (EPP) amplitude in 68 muscle fibres before addition of a CIC-1 inhibitor (black diamond) and 82 fibres in the presence of 20 μM NMD670 (open circle) during 12 Hz stimulation for 30 stimulations. With CIC-1 inhibition by NMD670, the EPP increased by more than 35% throughout a 12 Hz train of stimulation.

### Example 4: Intracellular Action Potential Measurement

The effect of CIC-1 inhibition on the capacity of neuromuscular junctions to excite muscle fibre APs in response to nerve stimulation in EAMG muscle fibres was next explored using both diaphragm and LAL muscle preparations. As when measuring EPPs an intracellular electrode was inserted in muscle fibres near their neuromuscular junction. To avoid muscular contraction disturbing the measurements, 100 μM of the myosin II inhibitor blebbistatin (Toronto Research Chemicals, CDN) was added to the bath solution before starting the experiment, and recordings were obtained from EAMG muscles before and after addition of 20 μM of NMD670. The nerve-stimulation protocol was similar to that used in the EPP protocol consisting of two trains of trigger pulses at 12 Hz and 30 Hz for 30 pulses with 10 seconds between the two trains. In EAMG muscles it was common to observe that nerve-stimulation did not trigger an AP, but after adding NMD670, AP excitation failures were less common. This was quantified by analyzing the probability that a given stimulus in the 30 Hz pulse train was able to trigger an AP before and after NMD670. Recordings were only included if the first nerve-stimulation triggered an AP in the muscle fibre.

As shown in FIG. 5, sustained action potential firing was compromised in nerve-muscle preparations from untreated EAMG animals (FIG. 5, middle trace) compared to healthy animals (FIG. 5, left trace). After addition of 20 μM NMD670, the success of repeated action potential excitations was improved markedly (FIG. 5, right trace and FIG. 6). Taken together, the restored EPP amplitude (FIG. 4) and improved action potential firing (FIG. 6) confirmed that CIC-1 inhibition enhances neuromuscular transmission in an EAMG model.

### Example 5: Force Measurement in Isolated Intact Nerve-Muscle Preparations

To determine whether enhanced neuromuscular transmission with CIC-1 inhibition could restore force in whole muscles from EAMG animals, experiments were conducted with soleus, extensor digitorum longus, and diaphragm muscles from EAMG rats. All muscles were mounted on force transducers (FORT 250, WPI instruments DE) in pre-warmed chambers (30° C.) containing 20 mL NKR each. The acquisition program Signal (version 6.4, Cambridge Electronics Design Ltd, Cambridge, UK) was used to control stimulation, delivered by an isolated stimulator (isostim 01D NPI electronics, DE) and for recording data via an analogue-digital converter (Micro 1401 Cambridge Electronics Design Ltd, Cambridge, UK). After equilibration in chambers, muscles were stretched to their optimal length and stimulated at a range of frequencies. For the duration of the experiment muscles were stimulated both directly and via the nerve at 12 V. This was accomplished by changing the stimulation pulse duration between 0.02 ms for nerve stimulation and 0.2 ms for direct stimulation of the muscle fibres. Muscles were stimulated every 10 min at 60 Hz for 1 second, either directly or via the nerve. To test for effect of NMD670 on force generation, different concentrations of NMD670 were added directly to the experimental chamber of the individual muscles.

FIG. 7 shows that force from isolated soleus muscle could be restored in EAMG rats after addition of 20 μM of NMD670 (right trace compared to middle trace). Further, in all muscle types tested, addition of NMD670 (between 20 and 40 μM) restored force generation (FIG. 8 and table 2) to levels that were close to the observations in muscles from healthy animals.

TABLE 2
Restoration of force in muscles from EAMG rats

| | Relative force area after addition of |
|---|---|
| Muscle type (number) | NMD670 (Initial area = 100) |
| Soleus (14) | 199 ± 17.1 |
| EDL (14) | 235.1 ± 25.0 |
| Diaphragm (12) | 120.2 ± 5.04 |

The data demonstrates that NMD670 can restore force in isolated muscle preparations from EAMG rats.

### Example 6: Force and EMG in Sedated EAMG Animals

An experimental setup was designed that enabled co-temporal measurements of electromyographic recordings of compound muscle action potentials (CMAP, EMG) and muscle force from triceps surae muscle in sedated and mechanically ventilated EAMG rats in response to nerve stimulation. Anaesthesia was introduced with a 1:1 mix of Fentanyl (Hypnorm) and Midazolam (Dormicum 5 mg/mL) (Hameln Pharma Plus gmbh, DE) at dose volume 1 mL/kg subcutaneously. Then, rats were intubated and mechanically ventilated to ensure adequate pulmonary gas exchange (Hallowell MicroVent 1 Rodent Anesthesia Ventilator, Dre Veterinary, KY, USA), and anaesthesia was maintained by mixing isoflurane (2-3%) in the ventilation gas. A tube was inserted through the oesophagus to the ventricle to allow PO dosing. The jugular vein was cannulated for acquisition of blood samples. The animals' core body temperature was continuously monitored and maintained at 37° C. by the heating pad upon which the animal was positioned.

When the animal was under stable anaesthesia, preparations for measuring EMG and force from a hind leg were made: The distal part of the triceps surae muscle was surgically exposed and a string was tied firmly to the Achilles' tendon after which the tendon was cut distally to the string. The string was connected to a force transducer (FORT 1000, World Precision Instruments, FL, USA). Two stimulation electrodes (Monopolar EMG Needle Electrode 25 mm×27 g, Chalgren, London, UK) were then inserted in the vicinity of the sciatic nerve to elicit nerve-stimulated contractile responses of the triceps surae muscle upon electrical stimulation of the nerve. The force generation elicited by stimulation was in turn recorded and quantified by the force transducer. Lastly, two EMG electrodes (subdermal needle electrode, Cadwell Kennewick, WA, USA) were placed subcutaneously with the active recording electrode placed distal to the knee joint over the proximal portion of the triceps surae muscle and the reference electrode was placed over the metatarsal region of the foot. The acquisition program (Signal version 6.4, Cambridge Electronics Design Ltd, Cambridge, UK) was used to control stimulation, delivered by an isolated stimulator (isostim 01D NPI electronics, DE) and to record the data via an analogue-digital converter (Micro 1401 Cambridge Electronics Design Ltd, Cambridge, UK).

After set-up, the sciatic nerve was stimulated with 10 pulses of 12 Hz (12-15 V) every 30 s. After 9 stimulations (5 min) the 12 Hz stimulation was substituted by 30 Hz stimulation for 1 second, and 9 stimulations later again finally substituted by 80 Hz stimulation for 1 second. This cycle was repeated until the end of the experiment. Muscle force data from 80 Hz stimulation was quantified as the Area Under the Curve (AUC) of the active force (g*s). For CMAP analysis the amplitude of the 4^{th} peak in trains of 12 Hz stimulation, and amplitudes of the 10^{th} peak in 30 and 80 Hz stimulation were used.

After set-up, the sciatic nerve was stimulated with 10 pulses at 12 Hz (12-15 V) every 30 s. After 9 stimulations (5 min) the 12 Hz stimulation was substituted by a single 30 Hz stimulation for 1 second, and 9 12 Hz stimulation trains later finally substituted by a single 80 Hz stimulation for 1 second. This cycle of 12, 30, and 80 Hz was repeated until the end of the experiment. Force elicited by 80 Hz stimulation was quantified as the Area Under the Curve (AUC) of the active force (g*s). For CMAP analysis, the amplitude of the 4^{th} peak in trains of 12 Hz stimulation, and amplitudes of the 10^{th} peak in 30 and 80 Hz stimulation were used.

To determine the pharmacokinetic (PK) and pharmacodynamic (PD) relationship for NMD670, different EAMG rats received single per oral doses of NMD670 ranging from 2 to 120 mg/kg, and plasma samples were collected into K₃EDTA coated 0.5 mL tubes (Sarstedt, DE) via jugular vein catheter at 10, 20, 30, 60 and for some animals at 120 minutes after compound administration. PK/PD relationship was determined by plotting recovery of EMG and force during 80 Hz stimulation against exposure of NMD670 in plasma.

Similar to isolated preparations, both EMG amplitude and nerve-stimulated muscle force were greatly depressed (FIG. 9, middle traces) in the EAMG compared with healthy animals (FIG. 9, left traces). Administration of NMD670 in EAMG animals caused rapid and dose-dependent restoration of both EMG and force (FIG. 9, right traces).

FIG. 10 depicts average muscle force (mean±SEM) from myasthenia gravis (EAMG) rats before (white) and after (grey) receiving NMD670 per oral (from 2 to 120 mg/kg), relative to muscle force from a healthy age matched rat. The figure shows that administration of NMD670 in EAMG rats caused rapid and dose-dependent restoration of muscle force.

### Example 7: Combined Mestinon and CIC-1 Inhibitor Administration in EAMG Rats

In EAMG animals with severe MG symptoms the effects of CIC-1 inhibitor and Mestinon (pyridostigmine) alone or in combination was assessed. Within 2-4 days after a rat had reached an EAMG score of 2, rats were allocated to treatment groups stratified using grip strength performance but also balanced on body weight such that grip strength relative to bodyweight ratio became comparable between groups. The study was blinded to both the experimenter and during subsequent analysis. Animals were subjected to grip strength test before compound administration, as described above and then dosed per oral with either 0.375 mg/kg pyridostigmine bromide (Mestinon) (CAS-no 101-26-8), 20 mg/kg NMD670 or a combination of both pyridostigmine and NMD670. Grip strength was tested 45 minutes post dosing. The average bodyweight across the groups before dosing was 190±9 grams, and the average grip strength before dosing was 1165±102 grams. The change in grip strengths was calculated for individual animals relative to the results obtained prior to administration of test article and then averaged.

As shown in FIG. 11 and Table 3, grip strength significantly increased after administration of NMD670, or a combination of NMD670 and Mestinon. Vehicle treatment did not affect grip strength.

TABLE 3
Increase in pulled force in grip strength test

| | Increase in Pulled | Significance Compared to |
|---|---|---|
| Group | Force (%) | Vehicle group* |
| Vehicle (n = 35) | 0.6 ± 1.7% | N/A |
| 0.375 mg/kg Mestinon (n =10) | 5.3 ± 3.8% | 0.76 |
| 20 mg/kg NMD670 (n = 17) | 14.5 ± 5.1% | 0.015 |
| 0.375 mg/kg Mestinon and | 24.2 ± 11.8% | 0.01^{#} |
| 20 mg/kg NMD670 (n = 5) | | |

*P value in one-way ANOVA
^{#}P value in one-way ANOVA compared to 0.375 mg/kg Mestinon was 0.12.

### Example 8: In Vivo 14-Day Dosing of NMD670 in EAMG Animals

Sixteen (16) rats with an EAMG clinical score of 2 were allocated to the study and stratified to one of the two treatment groups (vehicle vs NMD670, 8 rats per group). Stratification was based on rotarod endurance and grip strength performance with the aim to obtain similar average starting points for the two groups. Grip strength and Rotarod performance were measured prior to treatment (day 0) and on days 1, 4, 7, 9, 11 and 14 during treatment. The treatment was administered by per oral gavage. Dosing was twice daily with 20 mg/kg NMD670 or vehicle (sterile water) for 14 days unless the rat was subjected to premature termination due to reaching humane endpoints. The dose solutions for the treatment groups were blinded. Thus, experimenters and subsequent analysis were blinded as to which group received CIC-1 inhibitor and which group received vehicle. Full analysis of data and statistics were concluded before unblinding the data. Plasma samples were obtained on days 0, 1 and 14, with muscle samples obtained on day 14 at termination.

### Grip Strength

Grip strength was measured on a BIOSEB BIO-GT3 Grip strength meter (force transducer, S/N 180271). The rat was handled to allow all four paws to grab the mesh on the grip meter and the rat was then pulled 5 times by the base of the tail in the opposite direction of its body orientation, i.e. towards the operator and away from the grip meter. Care was taken to ensure that the force recorded reflected the grip strength of the rat and not the force of the pulls conducted by the operator. There was minimal pause between the 5 repeated pulls with recordings being separated only by the time of handling the rat (few seconds). Measurements were considered invalid if the rat let go with one or more limbs before the pull was initiated. Average values of the 5 pulls per test occasion were used for further data processing.

TABLE 4
Grip strength relative to bodyweight during 14-day chronic
dosing in EAMG rats receiving vehicle or NMD670.

| | | | P value from |
|---|---|---|---|
| Day in study | Vehicle Group | NMD670 group | Two-way ANOVA |
| 0 | 4.7 ± 0.29 | 4.8 ± 0.17 | P > 0.9999 |
| 1 | 4.3 ± 0.24 | 5.5 ± 0.46 | P = 0.1225 |
| 4 | 4.1 ± 0.29 | 5.4 ± .038 | P = 0.0629 |
| 7 | 4.3 ± 0.38 | 4.9 ± 0.26 | P = 0.7266 |
| 9 | 3.9 ± 0.30 | 5.0 ± 0.27 | P = 0.2100 |
| 11 | 3.9 ± 0.33 | 5.4 ± 0.41 | P = 0.0206 |
| 14 | 3.9 ± 0.33 | 5.1 ± 0.47 | P = 0.1010 |

As shown in FIG. 12 and table 4, rats that received NMD670 had increased grip strength over vehicle group throughout the period of treatment.

### Rotarod Test

Rotarod test was conducted on a Touchscreen Rotarod from Panlab (Model LE8305, Harward Apparatus). For testing the running performance, the rat was placed in one of 4 running lanes on the rotating rod that rats were challenged to run on. Prior to the first test on the rotarod on Day 0, the rat was allowed a 1-minute run at 4 rotations per minute (RPM), to familiarize with the apparatus. The rat was then allowed at least 5 minutes of rest before the test was conducted. To test the running performance, the rat was challenged with a protocol consisting of constantly accelerating speed from 4 to 40 RPM over 420 seconds. Test occasions consisted of 3 runs with at least 5 and no more than 15 minutes of rest between each run. Performance was quantified by the duration that the rat was able to remain on the rotating rod. Average values of the 3 runs per test occasion were used for further data processing.

As shown in FIG. 13 and table 5, rats that received NMD670 had higher endurance on rotarod over vehicle group throughout the period of treatment.

TABLE 5
Rotarod performance (latency to fall in seconds) during 14-
day chronic dosing in EAMG rats receiving vehicle or NMD670.

| | | | P value from Two- |
|---|---|---|---|
| Day in study | Vehicle | NMD670 | way ANOVA |
| 0 | 20.9 ± 6.3 | 28.5 ± 7.3 | P = 0.9478 |
| 1 | 37.9 ± 15.2 | 38.4 ± 8.7 | P > 0.9999 |
| 4 | 22.8 ± 7.6 | 37.8 ± 10.1 | P = 0.4613 |
| 7 | 18.0 ± 5.9 | 29.1 ± 8.8 | P = 0.7852 |
| 9 | 15.0 ± 6.0 | 27.5 ± 9.1 | P = 0.6753 |
| 11 | 10.3 ± 3.6 | 35.5 ± 12.6 | P = 0.0335 |
| 14 | 8.2 ± 2.6 | 19.0 ± 5.7 | P = 0.8122 |

### Bodyweight

Treated animals also showed an attenuation in loss of body weight (versus vehicle) during the study (FIG. 14). In both groups, early terminations were required when body weight declined below 80% of maximum body weight prior to disease induction. In the NMD670 treated group, 6 of the 8 treated rats completed the study while only 3 out of 8 vehicle treated rats completed the study. These findings thus support the notion that prolonged CIC-1 inhibition improves muscle function chronically in EAMG rats and this improves overall health status.

### Bioanalysis and LC-MS/MS

Concentrations of NMD670 were determined in both plasma and muscles from EAMG rats. For experiments involving anaesthetized rats, the blood samples were drawn using jugular vein catheter while for experiments with rats that were not anesthetized the blood samples were drawn by sublingual bleeding. Plasma samples were collected in K3EDTA coated 0.5 mL tubes (Sarstedt, DE). Muscle samples were obtained by surgically removing approx. 250 mg muscle tissue from the triceps surae, after the animal had been sacrificed, then immediately snap frozen in liquid nitrogen. Plasma concentrations of CIC-1 inhibitor NMD670 were determined by protein precipitation and liquid chromatography with mass spectrometric detection (LC-MS). NMD670 was used to prepare a 1 mg/mL solution in DMSO, adjusted for salt, which was then diluted to generate calibration spiking solutions (12.5, 25.0, 125.0, 250, 1250, 2500, 5000, 25000 and 50000 ng/mL) in DMSO from the primary stock solution. The resultant blank tissues were used for matrix calibration standards, which were prepared on ice on the same day as analysis was performed at 25.0, 50.0, 250, 500, 2500, 5000, 10000, 50000 and 100000 ng/mL by spiking blank plasma and 2:1 with NMD670 spiking solution.

To evaluate NMD670 concentrations in the muscle samples, muscles from animals exposed to NMD670 and blank tissues (muscle not exposed to compound or vehicle) were homogenized with three parts PBS per gram of tissue for a final processing dilution factor of fourfold. The resultant blank tissues were used for matrix calibration standards, which were prepared on ice on the same day as analysis was performed at 25.0, 50.0, 250, 500, 2500, 5000 ng/mL by spiking blank muscle tissue homogenate matrices at 1:2 with NMD670 spiking solution.

Subsequently, aliquots of the above prepared plasma or muscle samples, calibration standards and blanks were taken, and protein was precipitated by addition of 0.25% phosphoric acid in 100% methanol containing chloramphenicol at 75 ng/mL (1:40 or 1:10). Water was added at 9-fold and 1.5-fold the volume for plasma and tissue homogenate, respectively, to aid chromatography. The resultant matrix samples, matrix calibration standards and matrix blanks were thoroughly mixed, and protein precipitated at −20° C. overnight. The samples were centrifuged for 20 min at 2,500 g at 4° C. and 120 μL of the supernatant was transferred into a clean 96-well plate. All samples were processed independently in discrete batches containing appropriate matrix calibration standards. The analysis for each discrete batch was performed on an LC-MS system: Thermo Scientific™ Q Exactive™ Focus Orbitrap with a HESI-II electrospray source and UHPLC system using a Phenomenex Luna Omega C18 50 mm×2.1 mm analytical column (EMD Millipore) with a 1.6-μm pore size at 60° C. An injection volume of 4 μL was used for all samples and standards with a flow rate of 0.8 mL/min. The mobile phases consisted of the following: mobile phase A, 0.1% formic acid in MilliQ water; mobile phase B, 0.1% formic acid (v/v) in acetonitrile.

Mass spectrometry data were generated with negative electrospray ionization (ESI−) in full scan (150-10000 Da, 35000 resolution). Ions used for quantification were 309.97204 and 311.97006 for NMD670 and 321.00505 for Chloramphenicol (internal standard). Subsequent least-squares linear regression was performed on matrix calibration standards, and the matrix sample concentrations were interpolated from the appropriate matrix curve. All dilution factors were accounted for in final sample data with concentration of NMD670 expressed in ng/mL dividing by the molecular mass of compound.

### Example 9: Tablet Formulation

The immediate release tablets were formulated using standard excipients as described in table 6.

A standard high shear granulation process was developed. Microcrystalline cellulose was used as filler and binder excipient and maltodextrin was also applied as binder. The granulate contained 50-90% drug substance.

The granulate was dried and sieved and mixed with excipients to form a free-flowing blend. Silicified microcrystalline cellulose was added as a filler, Croscarmellose Sodium as a disintegrant and magnesium stearate as a lubricant. Silicified microcrystalline cellulose was found superior to microcrystalline cellulose as filler to achieve low weight variation. A single punch Diaf tablet press was used to manufacture the core tablets. The core tablets were film-coated with a standard white film-coating pre-mix (Opadry 03F180011 white) consisting of Hypromellose, polyethylene glycol 8000 and titanium dioxide. The term “Opadry White” refers to a composition coating prepared using Opadry® White obtained from Colorcon Pa, USA, in the form of the product sold as product code 03F180011 in 2022.

TABLE 6
Tablet Formulation

| | Product Strength | | | | |
|---|---|---|---|---|---|
| | Quantity Per Tablet (mg) | | | | |
| Substance | 50 mg | 100 mg | 200 mg | 300 mg | Function |

Active ingredient

| | | | | | |
|---|---|---|---|---|---|
| (2S)-2-[4-bromo-2-(1,2- | 50.0 | 100.0 | 200.0 | 300.0 | Drug substance |
| oxazol-3- | | | | | |
| yl)phenoxy]propanoic | | | | | |
| acid | | | | | |

Excipients of tablet core

| | | | | | |
|---|---|---|---|---|---|
| Silicified microcrystalline | 2.5-30 | 5-60 | 10-120 | 15-180 | Filler |
| cellulose | | | | | |
| Microcrystalline cellulose | 1-30 | 2-60 | 4-120 | 6-180 | Filler/Binder |
| Maltodextrin | 0.5-7 | 1-14 | 2-28 | 3-42 | Binder |
| Magnesium Stearate | 0.12-1.5 | 0.25-3 | 0.5-6 | 0.75-9 | Lubricant |
| Croscarmellose Sodium | 0.12-2.5 | 0.25-5 | 0.5-10 | 0.75-15 | Disintegrant |

Excipient of the coating

| | | | | | |
|---|---|---|---|---|---|
| Opadry 03F180011 white | 0.5-5 | 1-10 | 2-20 | 3-30 | Film-coating |
| | | | | | pre-mix |
| Total | 94.5 | 189 | 378 | 567 | |

15-30 mg/30-60 mg/90-180 mg Purified water (Ph. Eur), respectively was used and evaporated during processing of the NMD670 50 mg tablet, NMD670 100 mg tablet and NMD670 300 mg tablet, respectively.

### Example 10: Tablet Dissolution

Tablet dissolution was determined as follows.

TABLE 7
Chromatographic conditions

| Apparatus | HPLC system with UV/DAD detector | | |
|---|---|---|---|
| Column | XBridge BEH phenyl 2.5 μm 3.0 × 150 mm | | |
| Column temperature | 40° C. | | |
| Wavelength | 220 nm UV (DAD) detector | | |
| Flow | 0.6 mL/minute | | |
| Injection volume | 3 μL | | |
| Auto sampler | Ambient | | |
| temperature | | | |
| Mobile phase A (MFA): | Milli Q water/Acetonitrile/TFA, 80/20/0.05% | | |
| Mobile phase B (MFB): | Milli Q water/Acetonitrile/TFA, 5/95/0.05% | | |
| Mobile phase | Gradient | | |
| Gradient | Time | % MFA | % MFB |
| | 0.00 | 90 | 10 |
| | 1.00 | 90 | 10 |
| | 11.00 | 10 | 90 |
| | 12.00 | 10 | 90 |
| | 12.10 | 90 | 10 |
| | 16.00 | 90 | 10 |

| | |
|---|---|
| Runtime | 16.00 min |

TABLE 8
Dissolution System

| Apparatus | USP2 (Paddles) |
|---|---|
| Medium | Dissolution Media pH 6.8 |
| Media Volume | 900 ml |
| Paddle Speed | 75 RPM |
| Temperature | 37° C. |
| Sample Extraction Timepoints | 0, 15, 30, 45, 60, 90, 120 minutes |
| Filter | 10 μm |
| Dissolution profile | No replacement of dissolution |
| | media after withdrawal of sample |
| Sampling Volume | 1.5 mL |
| Number of Vessels | 6 |

Dissolution Media pH 6.8 was prepared by dissolving 27.3 g Na₂HPO₄·2H₂O and 4.9 g Citric Acid in 1 L Milli-Q water. The pH was measured and adjusted if necessary to pH 6.8.

900 mL of dissolution media pH 6.8 was accurately transferred into each dissolution vessel. The apparatus was assembled, and the dissolution media was heated to 37° C.±0.5° C. The T=0 point was extracted from each vessel prior to dropping the tablets into the dissolution vessels. One tablet was gently dropped into each dissolution vessel, and rotation of the paddle was immediately started. Samples were taken that the sample extraction timepoints and analysed using the HPLC method.

Not less than 80% of NMD670 is released from the tablets after a maximum of 60 minutes when performing release testing of the tablets.

### Example 11: Bioanalysis of Human Plasma Samples

### Sample Collection Method

Collect blood (3 mL recommended) onto wet ice (K₂EDTA tubes)
Immediately cool blood on crushed wet ice for maximum of 30 minutes
Harvest plasma as quickly as possible using refrigerated centrifuge by centrifuging at 2000 g for 10 minutes at 4° C.
Keep plasma on wet ice and immediately stabilise with 1:1 (v/v) addition of Water: Orthophosphoric Acid (100:2 v/v) prepared using ≥85% orthophosphoric acid and deionised water (e.g. 750 μL plasma+750 μL stabiliser).
Mix sample thoroughly.
Keep the stabilised plasma on wet ice during subsequent steps.
Divide the stabilised plasma into 5×250 μL aliquots in plastic cryotubes.
Store aliquots at nominal −70° C./−80° C. promptly.

### Sample Preparation Procedure

1. Thaw frozen calibration standards, QC samples, blank matrix and study samples on wet ice.
2. Vortex-mix all samples thoroughly.
3. On wet ice aliquot 150 μL of calibration standards, QC samples, study samples, and blanks into a 2 mL 96-well plate. For the reagent blank add the same volume of Water.
4. On wet ice add 25 μL of Methanol:Water (50:50) to wells containing blanks and the same volume of ISTD intermediate solution to all other wells.
5. On wet ice add 125 μL of Water to each well. Cap plate and vortex-mix (˜5 minutes, 1000 rpm).
6. Centrifuge plate (˜3000 g, 3 minutes, room temperature).
7. Condition SPE plate (Waters Oasis HLB pElution) with 250 μL Methanol.
8. Condition SPE plate with 250 μL of Water.
9. Load 250 μL of the sample onto the SPE plate (aspirate from the rim of the cone to avoid any debris collected at the bottom of the well). Pass through using minimum pressure.
10. Wash SPE plate with 250 μL of Water.
11. Wash SPE plate with 250 μL of Methanol:Water (5:95). Briefly increase pressure for one minute to ensure sorbent is partially dried.
12. Elute from SPE plate into a 1.2 mL 96-well plate using 30 μL of Methanol. Stand for one minute then pass through using absolute minimum pressure.
13. Elute again with another 30 μL of Methanol, collecting into the same plate. Briefly increase pressure for one minute to complete the elution.
14. Add 125 μL of Ammonium Formate 10 mM (aq):Formic Acid (100:0.3) to each well. Cap plate and vortex mix (˜1 minute, ≥1200 rpm).
15. Store plate refrigerated prior to analysis.

TABLE 9
HPLC Conditions

| | |
|---|---|
| HPLC instrumentation | Waters, Acquity Classic, UPLC Auxiliary |
| | pump, Switching valve |
| Analytical column | Astec CHIROBIOTIC T 250 × 4.6 mm, |
| | 5 μm (Supelco catalogue number |
| | 12024AST) |
| In-line filter | KrudKatcher ULTRA HPLC In-Line Filter |
| | 2 μm, Phenomenex (p/n AF0-8497) |
| Column temperature setting | 25° C. |
| Autosampler temperature | 5° C. |
| Mobile Phase A | Ammonium Formate 10 mM (aq):Formic |
| | Acid (100:0.3) |
| Mobile Phase B | Methanol |
| Back pressure (typical) | 3000 psi |
| Injection volume | 6-15 μL |

TABLE 10
Gradient Conditions

| Time (minutes) | A (%) | B (%) | Flow rate (mL/min) |
|---|---|---|---|
| Initial | 67.5 | 32.5 | 1.05 |
| 8.00 | 27.5 | 72.5 | 1.05 |
| 8.05 | 1.0 | 99.0 | 1.05 |
| 8.30 | 1.0 | 99.0 | 1.05 |
| 9.50 | 1.0 | 99.0 | 2.00 |
| 10.50 | 1.0 | 99.0 | 2.00 |
| 10.55 | 67.5 | 32.5 | 2.00 |
| 12.50 | 67.5 | 32.5 | 1.05 |

TABLE 11
Mass Spectrometer Parameters

| Mass spectrometer | Sciex API 5000 | |
|---|---|---|
| Ionization | ESI- | |
| Resolution | Q1: Unit | |
| | Q3: Unit | |
| IonSpray voltage | −4500 V | |
| Temperature | 550° C. | |
| Collision gas (CAD) | Nitrogen | Setting: 9 |
| Curtain gas (CUR) | Nitrogen | Setting: 30 |
| Nebulizing gas (GS1) | Nitrogen | Setting: 55 |
| Auxiliary gas (GS2) | Nitrogen | Setting: 65 |

| | |
|---|---|
| Entrance potential (EP) | −10 V |
| Pause time | 5 ms |
| Acquisition time | Delay time: 210 seconds delay |
| | Acquisition time after delay: 4.80 minutes |
| | Total time: 8.30 minutes |
| Cycle time | 13.3 min (injection start to next injection start) |

TABLE 12
Compound Detection Parameters

| | | | | | | Approximate |
|---|---|---|---|---|---|---|
| Com- | | Dwell | | | | RT and |
| pound | Transition | Time | DP | CE | CXP | window |
| Name | Monitored | (ms) | (V) | (V) | (V) | (min) |
| NMD670 | 310.0 → 237.9 | 60 | −80 | −19 | −12 | 5.80 min |
| ISTD1* | 315.0 → 242.9 | 60 | −80 | −19 | −12 | (4.06-7.54) |

*ITSD1 is NMD670-d₃.

### Example 12: Phase I Clinical Trial

Part A1 tested single doses of NMD670 in a double-blind, randomised, placebo-controlled, partial crossover and dose-escalating design in healthy male subjects (see the WHO International Clinicals Trials Registry Platform study reference NL8692). A total of nine dose levels were investigated in three cohorts of subjects. Each cohort consisted of nine subjects, each subject had three study sessions. Each subject received escalating doses of NMD670 on two occasions and placebo on one occasion, the order will be randomized in a cross-over fashion. Each dose level was randomized in a 6:3 ratio (active vs. placebo). See Table 13 for an overview of the randomization scheme.

TABLE 13
Randomisation Scheme

| Sequence | Period 1 | Period 2 | Period 3 |
|---|---|---|---|
| 1 | Placebo | Dose 2 | Dose 3 |
| 2 | Dose 1 | Placebo | Dose 3 |
| 3 | Dose 1 | Dose 2 | Placebo |

Dose escalation was stopped after an adverse event of myotonia of moderate intensity observed in one subject administered 1600 mg of NMD670 at dose level 7 (spontaneously and fully resolved within hours). Due to this temporary halt and partial unblinding of the study during dose level 7, a new randomization was necessary. After unblinding three subjects in dose level 7, the original randomization for dose 8 and 9 was changed in order to keep the study blinded. As subjects of cohort 3 only had 2 occasions left, the randomisation of the original design (3-way cross-over) was not possible without compromising the ratio of active and placebo treated subjects, and therefore the within-subject comparison which is important for the evaluation of PD markers. Therefore, for the remaining 2 doses, a full cross-over design investigating one previously tested dose level was used. The 9 subjects in cohort 3 were randomized to receive the study drug in level 8 and placebo in level 9, or vice versa. To determine the effect of food on the exposure of single oral doses of NMD670, dose level 5 was administered in both the fasted and fed condition. Subjects who received dose level 5 returned for a fourth visit in which they received dose level 5 (or matching placebo) in the fed condition, in the same randomization as the chosen dose level in the fasted condition.

Part A2 of the study investigated the safety, tolerability and pharmacokinetics of NMD670 in 8 healthy female subjects of non-childbearing potential, in a randomized, double-blind, placebo-controlled single dose administration of NMD670. Subjects received 800 mg NMD670. Subjects were randomized in a 6:2 ratio (active vs. placebo).

### Main Inclusion Criteria Healthy Volunteers (Part A)

1. Signed informed consent prior to any study-mandated procedure
2. Part A1: Healthy male subjects, 18 to 45 years of age, inclusive at screening.
3. Part A2: Healthy female subjects of non-childbearing potential, 18-65 years of age, inclusive at screening.
5. Body mass index (BMI) between 18 and 30 kg/m2, inclusive at screening, and with a minimum weight of 50 kg.
6. All males must practice effective contraception during the study and be willing and able to continue contraception for at least 90 days after their last dose of study treatment.
7. Has the ability to communicate well with the Investigator in the Dutch language and willing to comply with the study restrictions.

### Main Exclusion Criteria Healthy Volunteers (Part A)

1. Evidence of any active or chronic disease or condition that could interfere with or for which the treatment of might interfere with, the conduct of the study, or that would pose an unacceptable risk to the subject in the opinion of the investigator (following a detailed medical history, physical examination, vital signs (systolic and diastolic blood pressure, pulse rate, body temperature) and 12-lead electrocardiogram (ECG). Minor deviations from the normal range may be accepted, if judged by the Investigator to have no clinical relevance.
2. Clinically significant abnormalities, as judged by the investigator, in laboratory test results (including hepatic and renal panels, complete blood count, chemistry panel and urinalysis). In the case of uncertain or questionable results, tests performed during screening may be repeated before randomization to confirm eligibility or judged to be clinically irrelevant for healthy subjects.
3. Positive Hepatitis B surface antigen (HBsAg), Hepatitis C antibody (HCV Ab), or human immunodeficiency virus antibody (HIV Ab) at screening.
4. Systolic blood pressure (SBP) greater than 140 or less than 90 mm Hg, and diastolic blood pressure (DBP) greater than 90 or less than 50 mm Hg at screening.
5. Abnormal findings in the resting ECG at screening defined as:
   a. QTcF>450 or <300 msec for men and QTcF>470 or <300 msec for women
   b. Notable resting bradycardia (HR<45 bpm) or tachycardia (HR>100 bpm)
   c. Personal or family history of congenital long QT syndrome or sudden death;
   d. ECG with QRS and/or T wave judged to be unfavourable for a consistently accurate QT measurement (e.g., neuromuscular artefact that cannot be readily eliminated, arrhythmias, indistinct QRS onset, low amplitude T wave, merged T- and U-waves, prominent U waves);
   e. Evidence of atrial fibrillation, atrial flutter, complete branch block, Wolf-Parkinson-White Syndrome, or cardiac pacemaker
6. Use of any medications (prescription or over-the-counter [OTC]), within 14 days of study drug administration, or less than 5 half-lives (whichever is longer). Exceptions are paracetamol (up to 4 g/day) and ibuprofen (up to 1 g/day). Other exceptions will only be made if the rationale is clearly documented by the investigator.
7. Use of any vitamin, mineral, herbal, and dietary supplements within 7 days of study drug administration, or less than 5 half-lives (whichever is longer). Exceptions will only be made if the rationale is clearly documented by the investigator.
8. Participation in an investigational drug or device study (last dosing of previous study was within 90 days prior to first dosing of this study).
9. History of abuse of addictive substances (alcohol, illegal substances) or current use of more than 21 units alcohol per week, drug abuse, or regular user of sedatives, hypnotics, tranquillisers, or any other addictive agent.
10. Positive test for drugs of abuse at screening or pre-dose. Retesting is allowed at the discretion of the Investigator.
11. Alcohol will not be allowed from at least 24 hours before screening or pre-dose.
12. Smoker of more than 10 cigarettes per day prior to screening or who use tobacco products equivalent to more than 10 cigarettes per day and unable to abstain from smoking whilst in the unit.
13. Subjects will not be allowed to have excessive caffeine consumption, defined as >800 mg per day.
14. Any confirmed significant allergic reactions (urticaria or anaphylaxis) against any drug, or multiple drug allergies (non-active hay fever is acceptable).
15. Loss or donation of blood over 500 mL within three months (males) or four months (females) prior to screening or intention to donate blood or blood products during the study.
16. Any known factor, condition, or disease that might interfere with treatment compliance, study conduct or interpretation of the results such as drug or alcohol dependence or psychiatric disease.
17. History of trauma to the lower extremities or other conditions (most importantly neurological or muscle diseases) that, in the opinion of the investigator, could affect the electrophysiological measurements.
18. Excessive exercise within 7 days before study drug administration.
19. Clinically significant abnormalities in coagulation.

### Concomitant Medications for Healthy Volunteers (Part A)

No prescription medications and OTC medications were permitted within 14 days prior to study drug administrations, or less than 5 half-lives (whichever is longer), and during the course of the study.

No vitamin, mineral, herbal, and dietary supplements will be permitted within 7 days prior to study drug administrations, or less than 5 half-lives (whichever is longer), and during the course of the study.

Exceptions were paracetamol (up to 4 g/day) and ibuprofen (up to 1 g/day). Other exceptions were only be made if the rationale was clearly documented by the investigator. No exceptions were made for drugs that could influence the MVRC outcomes, such as sodium channel blockers, dantrolene, or anti-epileptic drugs. Use of substrates of CYP2C9, and CYP2C19 were prohibited. For CYP2C19 substrates, a) substrates exhibiting >5-fold increase in substrate AUC with CYP2C19 inhibitors: s-mephenytoin, omeprazole; or b) substrates exhibiting ≥2-fold but <5-fold increase in substrate AUC: diazepam, lansoprazole, rabeprazole, voriconazole were prohibited. For CYP2C9 substrates, a) substrates exhibiting ≥5-fold increase in substrate AUC with CYP2C9 Inhibitors: celecoxib; or b) substrates exhibiting ≥2-fold but <5-fold increase in substrate AUC: glimepiride, phenytoin, tolbutamide, warfarin were prohibited.

Use of any vaccine (initial or follow-up): there was a minimum period of 1 week between vaccination and screening; and 1 week between vaccination and dosing; until end of study.

### Tolerability/Safety Endpoints

The following endpoints were determined at time points indicated in the Schedule of Assessments.
Serious adverse events (SAEs) and adverse events (AEs) were collected throughout the study at every study visit.
Concomitant medication
Clinical laboratory tests
   Haematology
   Chemistry
   Urinalysis
   Coagulation
Vital signs
   Pulse Rate (bpm)
   Systolic blood pressure (mmHg)
   Diastolic blood pressure (mmHg)
   Respiratory rate
ECG
   Heart Rate (HR) (bpm), PR, QRS, QT, QTcF
24-hour Holter recording
Hand grip dynamometry
   Grip release profile; time profile from 100% maximum voluntary contraction (MVC) to 5% of the 100% MVC

### Results of Phase 1 (Part A)

The demographic characteristics of the 35 subjects enrolled in Part A of the Phase 1 study are presented in Table 14.

TABLE 14
Demographic Characteristics of 35 Subjects

| | | | |
|---|---|---|---|
| Age | Mean (SD) | 34.7 | (14.0) |
| Weight (kg) | Mean (SD) | 76.2 | (10.2) |
| Height (cm) | Mean (SD) | 178.5 | (9.8) |
| BMI | Mean (SD) | 24.0 | (3.1) |

| | | |
|---|---|---|
| Sex | Female (n) | 8 |
| | Male (n) | 27 |
| Race | Asian (n) | 3 |
| | Black (n) | 1 |
| | Mixed (n) | 2 |
| | Other (n) | 1 |
| | White (n) | 28 |

(SD)—standard deviation;
n—number of subjects

### Adverse Events

No serious or severe adverse effects were reported. There were no meaningful relationships between increases in NMD670 dose and the incidence of participants with AEs (FIG. 15). A total of 70 AEs were reported, of which 47 (67%) were at least possibly drug-related. Most common AEs reported (in >1 subject) are listed in FIG. 15. There were no relationships between increases in dose and the incidence of these individual AEs following administration of a single dose of NMD670, except for transient myotonia which was reported at the highest dose levels tested (1200 mg and 1600 mg). Most AEs were mild, except for one AE of myotonia (1600 mg), and tooth extraction (unrelated, 50 mg), which were moderate in intensity. There were no subject discontinuations.

### Pharmacokinetic Results

The pharmacokinetic results from the volunteers is given in table 15 below.

TABLE 15
Pharmacokinetic Results

| | AUC_{0-infinity} | Cₘₐₓ | T_{1/2} |
|---|---|---|---|
| | (Mean ± SD, | (Mean ± SD, | (mean ± SD, |
| Dose of NMD670 | h · ng/mL) | ng/mL) | hr) |
| 50 mg | — | 1900 ± 580 | — |
| 100 mg | 18500 ± 1770 | 3980 ± 1190 | 5.07 ± 0.63 |
| 200 mg | 36100 ± 5030 | 7780 ± 1130 | 4.6 ± 0.67 |
| 400 mg | 87700 ± 21400 | 20100 ± 7340 | 4.68 ± 0.49 |
| 800 mg fasted | 191000 ± 31500 | 36700 ± 15400 | 5.39 ± 0.82 |
| (male) | | | |
| 800 mg fed (male) | 180000 ± 27900 | 37300 ± 7420 | 5.02 ± 1.04 |
| 800 mg (female) | 256000 ± 56200 | 44400 ± 7480 | 5.55 ± 0.55 |
| 1200 mg | 358000 ± 176000 | 55000 ± 21700 | 6.11 ± 1.28 |
| 1600 mg | 826000 | 123000 | 4.48 |

*AUC₀₋ₗₐₛₜ (Mean ± SD, h · ng/mL).
“—” indicates the information is not available.

### Example 13: Phase IIA Clinical Trial

Part C of a phase I/IIA clinical trial was a double-blind, placebo-controlled, three-way cross-over comparison of two single oral doses of NMD670 in men and women with stable symptomatic myasthenia gravis (see the WHO International Clinicals Trials Registry Platform study reference NL8692). 12 patients were enrolled and completed the study. For each subject, the study consisted of three study periods during which subjects received either a single dose of NMD670 400 mg, NMD670 1200 mg, or placebo with a 7-day washout period between visits. On these study visits, subjects were asked to refrain from their regular dose of pyridostigmine (if applicable) from 14 h before dosing until after the last pharmacodynamic measurement on Day 1 (see Table 13: Visit and Assessment Schedule). The cross-over design allowed intra-individual comparison of drug effects versus placebo (see FIG. 16). A balanced design was applied to control for first-order carry-over effects.

### Main Inclusion Criteria Myasthenia Gravis Patients (Part C)

1. Signed informed consent prior to any study-mandated procedure
2. Male and female subjects, 18 and above years of age, inclusive at screening.
3. Diagnosis of myasthenia gravis, Myasthenia Gravis Foundation of America (MGFA) class I, II, III or IVa, based on characteristic muscle weakness and a positive AChR or muscle-specific tyrosine kinase (MuSK) antibody test (Jaretzki et al., Neurology, 2000, 55(1):16-23). Subjects with MGFA 0 using pyridostigmine (Mestinon) may be included, if muscle weakness is present when refraining from pyridostigmine (as assessed by a medical doctor based on an interview of the patient at screening).
4. Patients using steroids should be using a stable dose of steroids for at least 1 month before dosing, and the dose of steroids should be expected to remain stable for the duration of the study.
5. Body mass index (BMI) between 18 and 34 kg/m2, inclusive at screening, and with a minimum weight of 50 kg.
6. All women of child bearing potential and all males must practice effective contraception during the study and be willing and able to continue contraception for at least 90 days after their last dose of study treatment.
7. Has the ability to communicate well with the Investigator in the Dutch language and willing to comply with the study restrictions.
8. Must be able to cease the use of pyridostigmine as per study requirements, if applicable.

### Main Exclusion Criteria Myasthenia Gravis Patients (Part C)

1. Evidence of any active or chronic disease or condition apart from myasthenia gravis, that could interfere with, or for which the treatment of might interfere with, the conduct of the study, or that would pose an unacceptable risk to the subject in the opinion of the investigator (following a detailed medical history, physical examination, vital signs (systolic and diastolic blood pressure, pulse rate, body temperature) and 12-lead electrocardiogram (ECG)). Deviations from the normal range may be accepted, if judged by the Investigator to have no clinical relevance.
2. Clinically significant abnormalities, as judged by the investigator, in laboratory test results (including hepatic and renal panels, complete blood count, chemistry panel and urinalysis). In the case of uncertain or questionable results, tests performed during screening may be repeated before randomization to confirm eligibility or judged to be clinically irrelevant.
3. Positive Hepatitis B surface antigen (HBsAg), Hepatitis C antibody (HCV Ab), or human immunodeficiency virus antibody (HIV Ab) at screening.
4. Clinically significant abnormal findings in the resting ECG at screening, personal or family history of congenital long QT syndrome or sudden death, evidence of atrial fibrillation, atrial flutter, complete branch block, Wolf-Parkinson-White Syndrome, or cardiac pacemaker
5. Use of any medications that could influence the safety and conductance of the study (see prohibited concomitant medication) within 14 days of study drug administration, or less than 5 half-lives (whichever is longer).
6. Use of any vitamin, mineral, herbal, and dietary supplements within 7 days of study drug administration, or less than 5 half-lives (whichever is longer). Exceptions will only be made if the rationale is clearly documented by the investigator.
7. Participation in an investigational drug study (last dosing of previous study was within 90 days prior to first dosing of this study).
8. History of abuse of addictive substances (alcohol, illegal substances) in the past 5 years; or current use of more than 21 units alcohol per week, drug abuse, or regular user of sedatives, hypnotics, tranquillisers, or any other addictive agent
9. Positive test for drugs of abuse at screening or pre-dose (unless the test is positive for prescribed drugs). Retesting is allowed at the discretion of the Investigator.
10. Alcohol will not be allowed from at least 24 hours pre-dose.
11. Any confirmed severe allergic reactions against any drug, or multiple drug allergies (non-active hay fever is acceptable).
12. Loss or donation of blood over 500 mL within three months (males) or four months (females) prior to screening or intention to donate blood or blood products during the study.
13. Any known factor, condition, or disease that might interfere with treatment compliance, study conduct or interpretation of the results such as drug or alcohol dependence or relevant psychiatric disease (as judged by the Investigator).
14. History of trauma to the lower extremities or other conditions (most importantly neurological or muscle diseases) that, in the opinion of the investigator, could affect the electrophysiological measurements.
15. Clinically significant abnormalities in coagulation.
16. If a woman, pregnant, or breast-feeding, or planning to become pregnant during the study.
17. Gout or clinically relevant elevations in uric acid.

All medications (prescription and over-the-counter [OTC]) taken within 30 days of study screening were recorded. Treatment with pyridostigmine was allowed. Patients using steroids were using a stable dose of steroids for at least 1 month before dosing, and the dose of steroids were expected to remain stable for two months following dosing. Other current and recent (within 1 month prior to the screening) treatments were allowed, if judged by the investigator to have no clinical relevance. Drugs that could influence the MVRC outcomes were prohibited, such as, sodium channel blockers, dantrolene, or certain anti-epileptic drugs. Use of anticoagulants (vitamin K antagonists or DOACs) were not allowed. Use of substrates of CYP2C9, and CYP2C19 were prohibited. For CYP2C19 substrates, a) substrates exhibiting ≥5-fold increase in substrate AUC with CYP2C19 inhibitors: s-mephenytoin, omeprazole; or b) substrates exhibiting ≥2-fold but <5-fold increase in substrate AUC: diazepam, lansoprazole, rabeprazole, voriconazole were prohibited. For CYP2C9 substrates, a) substrates exhibiting ≥5-fold increase in substrate AUC with CYP2C9 Inhibitors: celecoxib; or b) substrates exhibiting ≥2-fold but <5-fold increase in substrate AUC: glimepiride, phenytoin, tolbutamide, warfarin were prohibited.

Use of any vaccine (initial or follow-up): there was a minimum period of 1 week between vaccination and screening; and 1 week between vaccination and dosing; until end of study.

### Treatment Duration

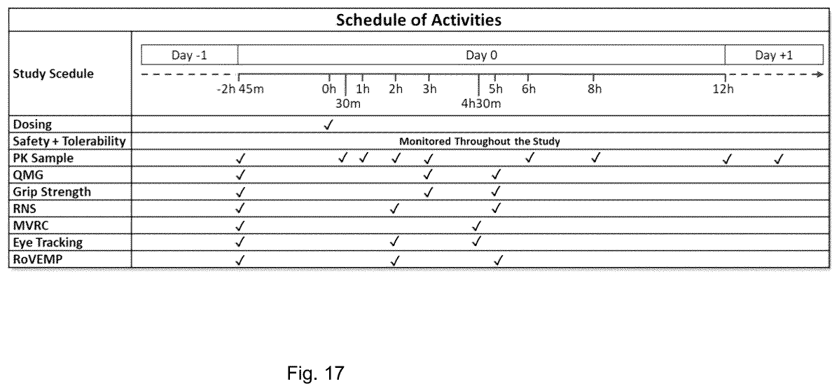

Part C was a double-blinded, placebo-controlled, three-way cross-over comparison of two single oral doses of NMD670 in men and women with stable symptomatic myasthenia gravis. 12 patients were enrolled. For each subject, the study consisted of three study periods during which subjects received either a single dose of NMD670 or placebo. On these study visits, subjects were asked to refrain from their regular dose of pyridostigmine (if applicable) for 14 hours before dosing. Single doses were administered in randomized order (400 mg free acid, 1200 mg free acid, or placebo p.o.), with a 7-day washout period between visits (FIG. 16). NMD670/placebo was administered with 240 ml of still water. The visit and assessment schedule is shown in FIG. 17.

### Quantitative Myasthenia Gravis Test

The Quantitative Myasthenia Gravis (QMG) total score (Barohn et al., Ann N YAcad Sci 1998, 841:769-72) is a validated clinical measure of sentinel muscle groups developed by the Myasthenia Gravis Foundation of America and the current clinical gold standard recommended for all prospective studies in MG. The QMG test is a physician-rated test using 13 assessments (see table 1), including facial strength, swallowing, respiratory, grip strength, and duration of time that limbs can be maintained in outstretched positions. Each of the 13 items is scored from 0 (none) to 3 (severe).

The total score can range from 0 to 39.

### Repetitive Nerve Stimulation (RNS)

RNS uses trains of 10 supramaximal electrical stimuli at 3 and 5 Hz using a Medelec Synergy 11.0. The optimal stimulation site on the skin is identified using inframaximal stimuli, after which the limit of supramaximal intensity is established. The working intensity is about 130% of that threshold. RNS is performed on the facial nerve/nasalis muscle combination. Techniques have been described previously (Niks et al., Muscle & Nerve, 2003, 28(2):236-238; Ruys-Van Oeyen et al., Muscle & Nerve, 2002, 26(2):279-282; Schumm et al., Muscle & Nerve, 1984, 7(2):147-151). The demographic characteristics of the patients in the Phase Ila clinical trial is shown in Table 16 and the results of the Quantitative Myasthenia Gravis test are given in table 17.

TABLE 16
Demographic Characteristics

| | | | |
|---|---|---|---|
| Age | Mean (SD) | 58 | (10.8) |
| BMI | Mean (SD) | 28 | (3.6) |
| Sex | Female (%) | 7 | (58%) |
| | Male (%) | 5 | (42%) |
| QMG total score | Mean (SD) | 9.0 | (3.6) |

| | | |
|---|---|---|
| at baseline | | |
| MGFA Clinical | I. Any ocular weakness | 1 |
| Classification | IIa. Mild, mostly limb/Axial | 9 |
| | IIb. Mild, mostly bulbar/respiratory | 1 |
| | IIIb. Moderate, mostly bulbar/ | 1 |
| | respiratory | |

TABLE 17
Results of the Quantitative Myasthenia Gravis test

| | 400 mg versus placebo | | | 1200 mg versus placebo | | |
|---|---|---|---|---|---|---|
| | Overall | 3-hour | 5-hour | Overall | 3-hour | 5-hour |
| CFB over placebo | −1.5 | −1.2 | −1.7 | −1 | −0.9 | −1.1 |
| LSM (p-value) | (p = 0.03) | (p = 0.12) | (p = 0.02) | (p = 0.14) | (p = 0.25) | (p = 0.14) |
| CFB over placebo | NA | −1.6 | −2.1 | NA | −1.2 | −1.5 |
| Arithmetic mean | | | | | | |

The NMD670 treatment arms have a Least Square Mean (LSM) change from baseline (CFB) improvement over placebo of 1.5 points (p=0.03). There was a change of 1.3 points at the 3-hour timepoint (p=0.12) and 1.7 points at the 5-hour timepoint (p=0.02) for the 400 mg treatment group. The Arithmetic Mean change for this group was 2.1 points at the 5-hour timepoint. The 1200 mg group had a Least Mean Square change improvement from baseline over placebo of 1.0 points (non-statistically significant) (0.9 points at the 3-hour timepoint and 1.1 points at the 5-hour timepoint).

A responder analysis was conducted to corroborate the clinical relevance of the QMG total score observations. Responders were defined as number (and proportion) of patients that showed an improvement equal or larger than 2 points from baseline compared to placebo (a clinically relevant improvement in patients with mild to moderate disease (Katzberg et al., Muscle Nerve, 2014, 49(5):661-5). In the 400 mg group at the 3-hour measurements, 6 of 12 patients achieved a 2-point or larger improvement in QMG total score, a 50% responder rate. The 400 mg group at the 5-hour and the 1200 mg group at 3- and 5-hour measurements showed that 5 of 12 patients achieved a 2-point or larger improvement in QMG total score, a 42% responder rate.

At the individual item level of the QMG study, symptoms including double vision, ptosis and dysarthria were improved for both the 400 mg and 1200 mg doses of NMD670 compared to placebo (non-statistically significant). Hand grip strength was also improved (see below).

The data demonstrates that administration of (2S)-2-[4-bromo-2-(1,2-oxazol-3-yl)phenoxy]propanoic acid (NMD670) can result in a reduction of the QMG total score in patients with myasthenia gravis and results in an improvement in the number and proportion of responders.

TABLE 18
Results of Right-hand grip strength test (an item of the QMG total score)

| | 400 mg versus placebo | | | 1200 mg versus placebo | | |
|---|---|---|---|---|---|---|
| | Overall | 3-hour | 5-hour | Overall | 3-hour | 5-hour |
| CFB over placebo | 1.4 kg | 2.4 kg | 0.4 kg | 2.8 kg | 2.9 kg | 2.8 kg |
| LSM (p-value) | (p = 0.19) | (p = 0.05) | (p = 0.76) | (p = 0.01); | (p = 0.02) | (p = 0.03) |
| CFB over placebo | NA | 2.4 kg | 0.4 kg | NA | 2.9 kg | 2.8 kg |
| Arithmetic mean | | | | | | |

Hand grip was evaluated with a Jamar hand grip dynamometer. An analysis of hand grip strength, an item of QMG score and therefore not an independent variable for the total score, was also performed (table 18). The change from baseline over placebo in hand grip strength was numerically positive for both the left and right hand at both doses and reached statistically significance for the right hand. The 1200 mg group had the largest Least Mean Square change improvement from baseline over placebo of 2.9 kg (p=0.02) at 3 hours and 2.8 kg (p=0.03) at 5 hours for right hand grip strength (table 16). The 400 mg group had a Least Mean Square change improvement from baseline over placebo of 2.4 kg at 3 hours and 0.3 kg at 5 hours (both non-statistically significant) for right hand grip strength.

A responder analysis was also conducted on the hand grip strength assessments. Responders were defined as number (and proportion) of patients that showed improvement equal to or larger than 10% over placebo. Responder rates between 25 and up to 67% were detected across dose levels and timepoints for right hand grip strength. Responder rates between 8 and up to 50% were detected across dose levels and time points for left hand grip strength.

The data demonstrates that administration of (2S)-2-[4-bromo-2-(1,2-oxazol-3-yl)phenoxy]propanoic acid (NMD670) can result in an increase in right-hand grip strength in patients with myasthenia gravis.

### Results of Repetitive Nerve Stimulation

Compound Muscle Action Potentials (CMAPs) were recorded from the nasalis muscle in response to nerve stimulation. Decrement refers to decline in CMAP amplitude during repeated stimulation and indicates neuromuscular transmission failure. No significant treatment effects and no trends of dose-dependent effects were found on CMAP decrement, across parameters investigated as continuous measurements. A responder analysis was also conducted to investigate the clinical relevance of the observed RNS changes in patients who presented with RNS decrement at baseline (more than 5% decrease in CMAP amplitude from the 1st to the 5th stimuli at 5 Hz stimulation). In the Placebo treatment arm 3 of 12 patients had decrement at baseline and from those 3 subjects, none recovered (less than 5% decrement) at the 3- and 5-hour measurements (0% responders). In contrast, in the 400 mg arm, 6 of 12 patients had decrement at baseline and from those 6 subjects, 5 recovered at the 3- and 5-hour measurements (83% responders). Similarly, in the 1200 mg arm, 3 of 12 patients had decrement at baseline and from those 3 subjects, 1 recovered at the 3- and 5-hour measurements (33% responders).

The data demonstrates that administration of (2S)-2-[4-bromo-2-(1,2-oxazol-3-yl)phenoxy]propanoic acid (NMD670) can result in a reduction of decrement in patients with myasthenia gravis.

### Pharmacokinetic Results

The pharmacokinetic results from the patients is given in table 19 below.

TABLE 19
Pharmacokinetic Results

| | | |
|---|---|---|
| Dose of NMD670 (mg) | 400 | 1200 |
| Cₘₐₓ (Mean ± SD, ng/mL) | 21,700 ± 5,970 | 76,700 ± 30,400 |
| AUC_{0-infinity} (Mean ± SD, | 93,700 ± 29,600 | 378,000 ± 113,000 |
| h · ng/mL) | | |
| Tₘₐₓ (median, hr) | 2.0 | 2.0 |
| T_{1/2} (median, hr) | 5.43 | 5.06 |

### Example 14: Phase 2b Clinical Trial

### 4. Study Design

### 4.1: Overall Design

This example describes a Phase 2b, randomised, double-blind, placebo-controlled study to evaluate the efficacy, safety, and tolerability of 3 dose levels of NMD670 in adult patients with AChR/MuSK-Ab+ MG.

The study will enrol male and female patients diagnosed with MG MGFA class II-IV and a QMG score of 11 or more and a Myasthenia Gravis Activities of Daily Living (MG-ADL) score of 6 or more at screening. Stratification for severity (patients with QMG_17) will be conducted.

The study duration will be up to 8 weeks for each participant including the following:
Screening (up to 4 weeks): visit 1
Baseline (day −1): visit 2
1-week treatment visit (day 7): visit 3
Telephone contact (day 14): visit 4 (remote)
End-of-treatment visit day 21: visit 5
Follow-up (7 days after end of treatment): visit 6

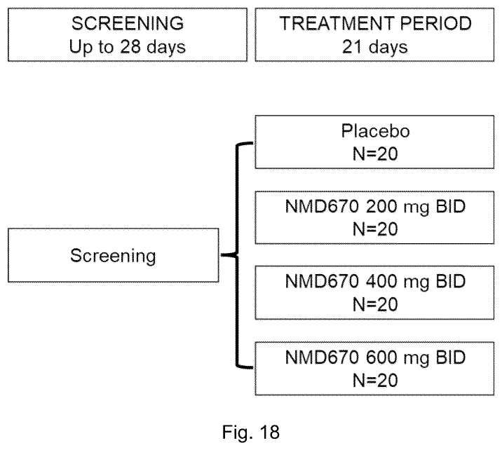

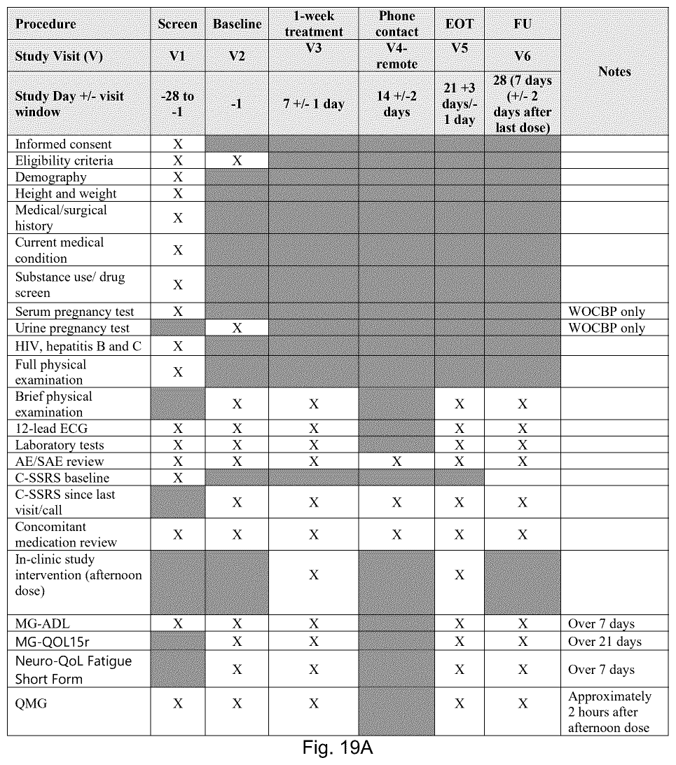

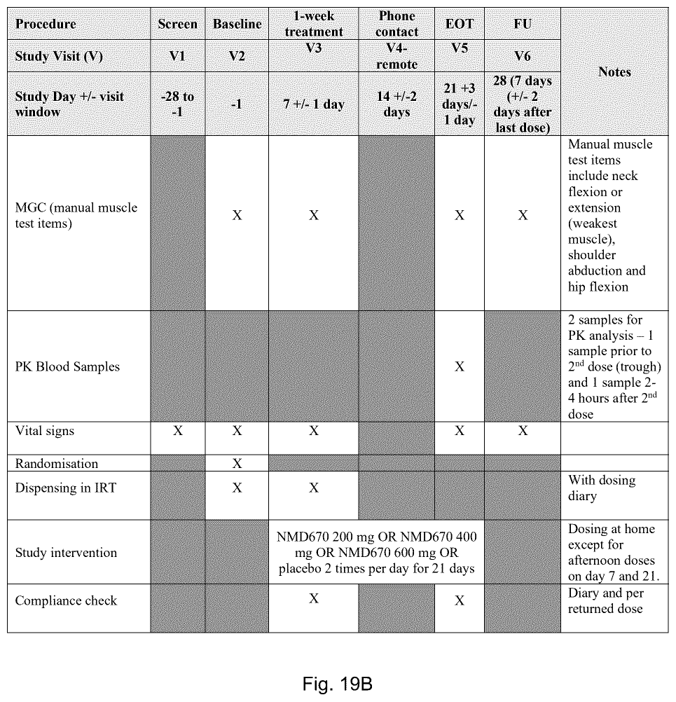

The study diagram is displayed in FIG. 18 and the schedule of activities (SoA) is provided in FIGS. 19A-19B.

After obtaining written informed consent from the participants, the Investigator will collect information and perform tests required to evaluate the eligibility of the participant, as listed in the SoA (FIGS. 19A-19B). Screening procedures may be conducted over several days within the 28-day screening period.

After screening, approximately 80 eligible participants will be randomised in a 1:1:1:1 ratio to receive one of the following treatments:
Placebo BID
NMD670 200 mg BID
NMD670 400 mg BID
NMD670 600 mg BID

Both the participant and Investigator will be blinded to treatment sequence assignment and will not know which treatment is given during each treatment period.

Participants will attend a baseline visit on the day before starting treatment and be dispensed with study treatment to be taken at home with a dosing diary. On the day after the baseline visit, they will start taking their treatment at home. They will attend a clinic visit on day 7 and be contacted by phone on day 14 and return to the site on the last day of treatment. During the visits to the clinic on day 7 and the last day of treatment (day 21), participants will take their morning dose at home before coming to the site and the second dose will be administered at the site during the visit. At End-of-treatment visit (day 21) two blood samples will be collected for PK analysis. One sample prior to the 2nd dose (trough) and one sample taken 2-4 hours after the 2nd dose around the expected Cmax.

After the end of treatment, participants will return to the site for a safety follow-up visit 6 days after the last dose of study treatment.

During the study, a SRC will review emerging patient data from the study, to ensure the safety of participants and the continued validity and scientific merit of the study.

TABLE 20
Objectives and Endpoints

| Objectives | Endpoints |
|---|---|
| Primary | |
| To assess dose response of | Change from baseline to day 21 in QMG |
| NMD670 on clinical efficacy | total score for NMD670 vs placebo |
| as rated by clinicians | |
| Key Secondary | |
| To assess clinical efficacy | Change from baseline to day 21 in QMG |
| of NMD670 on individual | total score for NMD670 vs placebo |
| dose levels vs. placebo, | Change from baseline to day 21 in MG- |
| e.g., high dose level | ADL total score for NMD670 vs placebo |
| vs placebo | Change from baseline to day 21 in MGC |
| | total score for NMD670 vs placebo |
| | Change from baseline to day 21 in MG- |
| | QOL15r for NMD670 vs placebo |
| | Change from baseline to day 21 in |
| | Neuro-QoL Fatigue Short Form |
| Secondary | |
| To assess dose response | Change from baseline to day 21 in MG- |
| of NMD670 on clinical | ADL total score for NMD670 vs placebo |
| efficacy as rated by | Change from baseline to day 21 in MGC |
| clinician and patients | total score for NMD670 vs placebo |
| | Change from baseline to day 21 in MG- |
| | QOL15r for NMD670 vs placebo |
| | Change from baseline to day 21 in |
| | Neuro-QoL Fatigue Short Form |
| To assess responder | Proportion of patients with clinically |
| rates on clinical efficacy | relevant changes from baseline on QMG |
| as rated by clinician | total score for NMD670 vs placebo |
| and patients | Proportion of patients with clinically |
| | relevant changes from baseline on MG- |
| | ADL total score for NMD670 vs placebo |
| | Proportion of patients with clinically |
| | relevant changes from baseline on MGC |
| | total score for NMD670 vs placebo |
| | Proportion of patients with clinically |
| | relevant changes from baseline on MG- |
| | QOL15r total score for NMD670 vs |
| | placebo |
| To assess safety and | AEs, physical examinations, clinical |
| tolerability of NMD670 | laboratory parameters, vital signs, ECG, |
| over 21 days compared | C-SSRS |
| with placebo | |
| Exploratory | |
| To explore drug exposure | Plasma levels of NMD670. |
| of NMD670 after 21-days | |
| To assess dose response of | Change from baseline to day 21 in QMG |
| NMD670 on clinical efficacy | total score for NMD670 vs placebo |
| and individual dose levels | Change from baseline to day 21 in MG- |
| vs. placebo in subgroups | ADL total score for NMD670 vs placebo |
| interest, e.g., patients | Change from baseline to day 21 in MGC |
| with antibodies against | total score for NMD670 vs placebo |
| MuSK, and AChR | Change from baseline to day 21 in MG- |
| | QOL15r total score for NMD670 vs |
| | placebo |
| | Change from baseline to day 21 in |
| | Neuro-QoL Fatigue Short Form |
| To assess the time to | Change from baseline to day 7, and 21 in |
| clinical efficacy of | QMG, MG-ADL, MGC and total score for |
| NMD 670 | NMD670 vs placebo |

Abbreviations:
AE = adverse event;
C-SSRS = Columbia-Suicide Severity Rating Scale;
ECG = electrocardiogram;
MG-ADL = Myasthenia Gravis Activities of Daily Living;
MGC = Myasthenia Gravis Composite;
MG-QOL15r = Myasthenia Gravis Quality of Life 15 Scale Item revised;
QMG = Quantitative Myasthenia Gravis Score

### Estimand:

The primary clinical question of interest for the objective of clinical efficacy as rated by clinician is:
Is there a non-flat exposure-response pattern in change from baseline to Day 21 of QMG total score in adult patients with Myasthenia Gravis treated with placebo versus NMD670?

Specifically, the estimand has the following attributes:
Population:
   The target population of interest are male and female patients aged ≥18 years, diagnosed with MG defined as MGFA class II-IV, a QMG score of 11 or more and an MG-ADL score of 6 or more at screening. These are all defined by the inclusion and exclusion criteria
Patient-Level Endpoint:
   The change from baseline to day 21 in QMG score will be derived for each patient. The baseline value will be the QMG score closest (but before) first dose
Intercurrent Events
   Anticipated Intercurrent Events include treatment discontinuation and treatment noncompliance. The treatment policy strategy will be used with respect to these types of events; thus, all available data will be used
Population Level Summary
   Point estimates (and confidence intervals) of the parameters from the dose-response model will be used to characterise the dose-response relationship. Placebo will be considered as dose 0 in this model
   The primary efficacy endpoint will be analysed in all randomly assigned patients, by means of the treatment assigned for analysis (Full Analysis Set)

### 4.2: Scientific Rationale for Study Design

The proposed primary efficacy endpoint for this study is change from baseline to day 21 in QMG score to be assessed in the intention-to-treat population.

The QMG total score was selected as the primary variable of the study to assess the effect of NMD670 after chronic dosing. The QMG score for disease severity is a validated clinician rated clinical measure of sentinel muscle groups developed by the MGFA and the gold standard recommended for prospective studies in MG by the MGFA task force (Jaretzki et al., Ann Thorac Surg. 2000 July; 70(1):327-34; Bedlack et al, Neurology. 2005 Jun. 14; 64(11):1968-70).

The total QMG score ranges from 0 to 39, where higher scores indicate greater disease severity. The QMG is designed to evaluate symptoms of weakness and fatigue geared towards MG of the following domains/muscle groups: ocular (2 items), facial (1 item), bulbar (2 items), gross motor (6 items), axial (1 item), and respiratory (1 item). Inter-rater reliability is high and construct validity and correlation with other (patient-rated and clinician-rated) MG outcome measures is good (Barnett et al, Neuromuscul Disord. 2013 May; 23(5):413-7; Barnett et al, J Clin Neuromuscul Dis. 2012 June; 13(4):201-5; Wolfe et al, Muscle Nerve. 2008 November; 38(5):1429-1433; Howard et al Muscle Nerve. 2017 August; 56(2):328-330; Vissing et al Muscle Nerve. 2018 August; 58(2):E21-E22; de Meel et al J Neuromuscul Dis. 2020; 7(3):297-300; McPherson et al Muscle Nerve. 2020 August; 62(2):261-266).

Based on the mechanism of action of enhancement of muscle strength and function through improved transmission of signals from nerve to muscle for NMD670, the QMG score was an exploratory outcome in the phase 1 study of NMD670 in 12 patients with MG (see Example 13). In this study, administration of single doses of NMD670 in patients with MG was associated with clinically significant improvements in the QMG total Score and with up to 50% of the patients meeting responder criterion, thus, confirming the sensitivity of the QMG score to detect improvement in symptoms with NMD670 and supporting the proposal of QMG total score as primary outcome measure for the proposed phase 2 dose range finding study.

Secondary efficacy outcomes are change from baseline to day 21 in Myasthenia Gravis Activities of Daily Living response (MG-ADL), Myasthenia Gravis Composite (MGC) score, Myasthenia Gravis Quality of Life15-item scale revised (MG-QoL15r) score, and Neuro-QoL Fatigue Short Form. The MG-ADL is an 8-item patient-reported outcome measure that assesses MG specific symptoms and their impact on daily activities. The MG-QOL15r is a 15-item patient-reported outcome measure assessing physical, psychological, and social domains commonly impacted by MG. The MGC assesses disease specific symptoms and examination findings derived from the MG-ADL (patient reported), QMG and Manual Muscle Test (MMT) (clinician determined). It consists of 10 weighted items. The Neuro-QoL Fatigue Short Form is a patient reported outcome that assesses fatiguability on 8 items on a 5-point scale. Thus, the study will provide information for selection of appropriate outcome measures for subsequent pivotal studies of NMD670 in MG.

### 4.3: Justification for Dose

NMD670 will be administered at 200, 400 or 600 mg BID to give total daily doses of 400, 800 or 1200 mg, respectively. These dose levels have been selected to explore the therapeutic dose range of twice a day administered NMD670 in MG patients based on the highest single dose level tested of 1200 mg in patients with MG in the phase 1 FIH clinical study.

Pharmacokinetic parameters for NMD670 at the dose level of 1200 mg once a day (QD) were found to be similar between healthy subjects and patients with MG, and the PK parameters were shown to be approximately linear and predictable. NMD670 is mostly eliminated in 24 hours (half-life of ˜5.0 hrs.) with no meaningful accumulation between single and multiple dosing.

The majority of AEs in the phase 1 FIH study were mild in intensity and no AEs were severe. No serious adverse reactions were reported in the phase 1 study. Myotonia of moderate severity was reported in 1 healthy subject receiving a single dose of 1600 mg. This adverse reaction (AR) resolved spontaneously, and no further subjects were dosed at 1600 mg. At 1200 mg QD, the presence of myotonic symptoms was observed in 2 healthy subjects and was mild in intensity and resolved spontaneously within a few hours. Myotonia is considered to be exaggerated on-target CIC-1 pharmacology and based on this clinical finding the maximum tolerated dose of NMD670 was determined to be 1200 mg QD in healthy subjects. Notably, myotonia was not observed in patients with MG. The maximum tolerated dose of NMD670 for this patient population could, therefore, be higher.

Taking into consideration there were no treatment-related adverse effects at the 1200 mg doses in MG patients in the phase 1 FIH clinical study, administration to a dosage of 600 mg bid for a duration of 21 days is considered safe.

Furthermore, the dosage of 200 mg, 400 mg and 600 mg BID are predicted to maintain pharmacologically active concentrations for approximately 13 hours, if administered 6 hours apart, providing a potentially therapeutic effect for the entire waking day.

### 4.4: End-of-Study Definition

The end of the study is defined as the date of the last visit of the last participant in the study globally.

A participant is considered to have completed the study if the participant has completed all periods of the study including the follow-up (visit 6).

### 5: Study Population

Prospective approval of protocol deviations to recruitment and enrolment criteria, also known as protocol waivers or exemptions, is not permitted.

The study population will consist of adult participants with MG with antibodies against AChR or MuSK. Participants must be able to provide written consent and meet all the inclusion criteria and none of the exclusion criteria. Patients >75 years are not eligible to participate to avoid exposing patients of advanced age to an experimental drug substance, as they are known to have larger physiological variations on pharmacokinetic and pharmacodynamic variables, higher rate of comorbidities and polypharmacy, all of which could confound the interpretation of the study results. It is important to note that future studies will include this patient population.

### 5.1: Inclusion Criteria

Participants are eligible to be included in the study only if all of the following criteria apply:

### Age and Gender

1. Participant must be a male or female being 18 to 75 years (both included), at the time of signing the informed consent.

### Type of Participant and Disease Characteristics

2. Diagnosis of MG, MGFA class II, III or IV.
3. Documented positive AChR or MuSK antibody test.
4. Patients with QMG total score of 11 or more and MG-ADL score of 6 or more at screening.
5. Patients on treatment with steroids are required to be on a stable dose for 4 weeks prior to screening and the dose should be expected to remain stable for the duration of the study.
6. Patients on treatment with azathioprine, ciclosporin, methotrexate, mycophenolate mofetil, and tacrolimus (or any other off-label treatment for MG) are required to have been treated for at least 6 months prior to screening and to be on a stable dose for a least 2 months prior to screening and the dose should be expected to remain stable for the duration of the study.
7. Patients on ACh-esterase inhibitors are required to be on a stable dose for 2 months prior to screening and the dose should be expected to remain stable for the duration of the study. In addition, patients should withhold Ach-esterase inhibitor treatment on the testing days; at the baseline, 1-week treatment visit (day 7), and the end of treatment visit (day 21).
8. Participant must be able to swallow tablets.

### Weight

9. Body mass index (BMI) between 18 and 34 kg/m2, inclusive, at screening, and with a minimum weight of 40 kg.

### Contraceptive/Barrier Requirements

10. Contraceptive use by men and women must be consistent with local regulations regarding the methods of contraception for those participating in clinical studies

### Informed Consent

11. Participant is capable of and has given signed informed consent.

### 5.2: Exclusion Criteria

Participants are excluded from the study if any of the following criteria apply:

### Medical Conditions

1. Known medical or psychological condition(s) or risk factor that, in the opinion of the Investigator, might interfere with the patient's full participation in the study, pose any additional risk for the patient, or confound the assessment of the patient or outcome of the study.
2. Active or untreated thymoma, a history of thymic carcinoma or thymic malignancy (unless deemed cured by adequate treatment with no evidence of recurrence for 5 years or more before screening), a history of thymectomy in the 6 months before screening.
3. Participant with a clinical diagnosis of gout, or with serum uric acid >6.5 mg/dL at screening.
4. Study participant has 12-lead ECG with findings considered to be clinically significant upon medical review. The clinical significance of the findings needs to be assessed by the Investigator to determine eligibility, and any questions regarding eligibility to participate in the study will have to be addressed with the Medical Monitor.
5. Participant with any of the following abnormalities in QT interval corrected for heart rate using Fridericia's correction (QTcF) at screening:
   a. QTcF>450 msec for male participants without bundle branch block.
   b. QTcF>470 msec for female participants without bundle branch block.
   c. QTcF>480 msec in participants with bundle branch block.
6. Participant with any of the following:
   a. Abnormal liver function test defined as total bilirubin>1.5×upper limit of normal (ULN) (except for participants with known Gilbert's syndrome).
   b. Current or chronic history of liver disease including (but is not limited to) hepatitis virus infections, drug- or alcohol-related liver disease, non alcoholic steatohepatitis, autoimmune hepatitis, hemochromatosis, Wilson's disease, a 1 antitrypsin deficiency, primary biliary cholangitis, primary sclerosing cholangitis, or any other liver disease considered clinically significant by the Investigator.
   c. Known hepatic or biliary abnormalities (with the exception of Gilbert's syndrome or asymptomatic gallstones).
7. Participant with laboratory test abnormalities at screening considered clinically significant by the Investigator.
8. Participant with breast cancer within the past 10 years or lymphoma, leukaemia, or any malignancy within the past 5 years. An exception of this 5-year requirement is basal cell or squamous epithelial carcinomas of the skin that have been resected with no evidence of metastatic disease.
9. Participants with ongoing significant psychiatric disorder (e.g., uncontrolled depression or anxiety).
10. Participants with positive drug screen (cocaine, heroin, opiates) at screening or marijuana drug abuse [as determined by positive drug screen and judged by the investigator]) at screening.
11. Participants with positive human immunodeficiency virus (HIV) antibody test.
12. Participants with positive serology for hepatitis B (unless due to vaccination, or resolved natural infection, or passive immunization, as confirmed with the medical monitor).
13. Participants with positive hepatitis C antibody.
14. Participants with a clinically significant history of allergic conditions (including drug allergies and anaphylactic reactions) or hypersensitivity to any component of the investigational medicinal product (IMP).

### Prior/Concomitant Therapy

15. Use of intravenous or subcutaneous immunoglobulin or plasma exchange 4 weeks prior to screening.
16. Use of rituximab 6 months prior to screening.
17. Use of FcRn-antagonists or C5 inhibitors. For Efgartigimod, Rozanolixizumab, Eculizumab and Zilucoplan, the patients should have received the last dose at least 3 months prior to screening. For Ravulizumab the patients should have received the last dose at least 9 months prior to screening.
18. Participants who have received any prohibited medication within 30 days (or 5 half-lives of the medication, whichever is longer), or are likely to require treatment with prohibited medications during the study.
Prohibited medications are listed in Section 6.9 and include drugs affecting neuromuscular transmission (such as anticholinergic drugs), drugs showing relevant effect on CIC-1 channel, and drugs with potential drug-drug interactions with NMD670.

### Prior/Concurrent Clinical Study Experience

19. Participants received treatment with investigational medical product (IMP) within 30 days (or 5 half-lives of the medication, whichever is longer) prior to day 1.

### Other Exclusion Criteria

20. Participants with history of poor compliance with relevant MG therapy.
21. Female patients who plan to become pregnant or are currently pregnant or breastfeeding.

### 5.3: Lifestyle Considerations

1. Participants should take their treatment with a regular glass of water.
2. During the treatment period, participants should be careful to drink sufficiently and remain properly hydrated. On average it might mean 2 liters of water a day for an adult person.
3. Participants should avoid changing their daily activities during the study. 4. Participants should avoid out of the ordinary, strenuous, or excessive physical activity (e.g., any form of heavy lifting or fitness training) for 7 days prior to each site visit, while maintaining usual ambulatory activities. Participants will be asked to maintain minimal physical activities on the day preceding and the morning of each site visit.
5. Alcohol abuse is not allowed for the duration of the study and patients should abstain from alcohol for 24 hours prior to each site visit.

### 5.4: Screen Failures

A screen failure occurs when a participant who has consented to participate in the clinical study is not subsequently assigned to study intervention. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, reason for screen failure (e.g., eligibility requirements failed), and documentation of any medical occurrences that qualify as SAEs).

Individuals who do not meet the criteria for participation in this study (screen failure) may be rescreened at the discretion of the investigator. Individuals may be rescreened no more than 2 times. The individual is eligible for enrolment if the results of repeated screening assessments meet eligibility criteria. Rescreened participants should sign a new informed consent form (ICF) and be assigned a new participant number for every screening/rescreening event.

### 5.4.1: Screening and Enrolment Log and Participant Identification Numbers

The participant's enrolment will be recorded in the Screening and Enrolment Log. Upon enrolment, each participant will receive a unique participant identification number. Participant numbers must not be re-used for different participants.

### 6: Study Intervention(s) and Concomitant Therapy

Study interventions are all pre-specified, investigational, and non-IMP(s), medical devices, and other interventions (e.g., surgical and behavioural) intended to be administered to the study participants during the study conduct.

### 6.1: Study Intervention(s) Administered

TABLE 21
Study Intervention(s) Administered

| Intervention | NMD670 | Placebo |
|---|---|---|
| Dosage | tablet | tablet |
| Formulation | | |
| Unit Dose | 200-mg tablets | matching tablets with |
| Strength(s) | | no active treatment |
| Dosage | 200 mg BID | 3 tablets BID |
| Level(s) | 2 × 3 tablets (1 tablet | 2 × 3 tablets (3 |
| | NMD670 and 2 tablets placebo) | tablets placebo) |
| | 400 mg BID | |
| | 2 × 3 tablets (2 tablets | |
| | NMD670 and 1 tablet placebo) | |
| | 600 mg BID | |
| | 2 × 3 tablets (3 tablets | |
| | NMD670) | |
| Route of | oral | oral |
| Administration | | |
| Use | experimental | placebo-comparator |
| IMP or NIMP | IMP | IMP |

| | |
|---|---|
| Sourcing | Provided centrally by the Sponsor |
| Packaging and | The IMPs (NMD670 and placebo) will be packaged and |
| Labelling | labelled as required per country requirement. |

Abbreviations: BID = twice a day; IMP = investigational medicinal product.

### 6.1.1: Administration of Study Intervention

Placebo or NM0670 tablets should be swallowed as whole tablets together with a regular glass of water.

One day before the treatment period, participants will attend a baseline visit (visit 2) at the study site and be dispensed with tablets to be taken at home. The next day, participants will start their treatment at home for 21 days at approximately the same time every day in the morning and 6 hours later in the afternoon (within 5 to 8 hours after the morning dose). Participants who miss a morning and/or an afternoon dose should not attempt to add or recover the missed dose neither at a later time nor the next day.

On day 7 (visit 3) and on the end of treatment day 21 (visit 5), participants will attend a visit to the study site. They will be asked to take the morning dose at home and the second dose will be administered at the site at approximately noon. If participants forget to take the morning dose at home before the visit, it will be administered at the site.

During visit 2 (baseline) participants will be dispensed a sufficient number of tablets covering the first treatment week. At visit 3 (day 7) participants should be asked to bring remaining tablets and packing boxes to the site. Participants will then be dispensed a sufficient number of tablets covering the remaining treatment period. At visit 5 (end of treatment) participants should be asked to bring remaining unused tablets and packing boxes to the site.

### 6.2: Preparation, Handling, Storage, and Accountability

The Investigator or designee must maintain a log to confirm appropriate conditions (e.g., temperature) have been maintained during transit for all study intervention received, and any discrepancies are reported and resolved before use of the study intervention.

All supplies of study intervention must be stored in accordance with the manufacturer's instructions in a temperature-controlled environment between 15 and 25° C. The study intervention will be stored in a securely locked area, accessible to authorised persons only, until needed for dosing.

Only participants enrolled in the study may receive study intervention, and only authorised site staff may supply, prepare, or administer study intervention. All study interventions must be stored in a secure, environmentally controlled, and monitored (manual or automated) area in accordance with the labelled storage conditions with access limited to the Investigator and authorised site staff.

The Investigator or authorised site staff is responsible for study intervention accountability, reconciliation, and record maintenance (e.g., receipt, reconciliation, and final disposition records).

### 6.3: Assignment to Study Intervention

All participants will be centrally randomised using an interactive response technology (IRT). Each participant will be assigned a unique number that encodes the participant's assignment to 1 of the 4 treatment arms of the study according to the randomisation schedule.

Study intervention will be administered/dispensed at the study visits as summarised in the SoA (FIGS. 19A-19B).

Returned study tablets should not be re-dispensed to the participants.

### 6.4: Blinding/Masking

This is a double-blind study in which participants, Investigators, site staff, Sponsor, monitors, and members of the Safety Review Committee (SRC) are blinded to study intervention. The IRT will be programmed with blind-breaking instructions. In case of an emergency, the Investigator has the sole responsibility for determining if unblinding of a participant's study intervention assignment is warranted. Participant safety must always be the first consideration in making such a determination. If the Investigator decides that unblinding is warranted, the Investigator may, at the Investigator's discretion, contact the Sponsor to discuss the situation prior to unblinding a participant's study intervention assignment unless this could delay emergency treatment for the participant. If a participant's study intervention assignment is unblinded, the Sponsor must be notified within 24 hours of this occurrence. The date and reason for the unblinding must be recorded.

The Sponsor (or designee) may unblind the intervention assignment for any participant with an SAE. If the SAE requires that an expedited regulatory report be sent to one or more regulatory agencies, a copy of the report, identifying the participant's intervention assignment, may be sent to Investigators in accordance with local regulations and/or Sponsor policy.

### 6.5: Study Intervention Compliance

When participants self-administer study intervention(s) at home, they will be instructed to record the time of each morning and afternoon dosing in a diary every day and return unused tablets to the study site. Compliance with study intervention will be assessed by the Investigator when participants return to the site on the last day of the treatment period, by reviewing the diary entries and counting returned tablets. Compliance will be documented in the source documents and relevant form. Deviation(s) from the prescribed dosage regimen should be recorded.

When participants are dosed at the site, they will receive study intervention directly from the Investigator or designee, under medical supervision. The date and time of each dose administered in the study site will be recorded in the source documents. A record of the quantity of tablets dispensed to and administered by each participant must be maintained and reconciled with study intervention and compliance records. Intervention start and stop dates, including dates for intervention delays and/or dose reductions will also be recorded.

### 6.6: Dose Modification

No dose modifications are planned during the study.

6.7: Continued Access to Study Intervention after the End of the Study

After completing the study, participants will stop receiving study intervention.

### 6.8: Treatment of Overdose

For this study, any dose of NMD670 greater than 600 mg BID (i.e., 1200 mg per day) will be considered an overdose.

In the event of an overdose, the Investigator should:
Evaluate if an urgent medical consultation is needed
Evaluate if an unscheduled study visit is needed
In all cases, closely monitor the participant for any AE/SAE and laboratory abnormalities as medically appropriate until, at least, the next scheduled follow-up visit.
Document the quantity of the tablets taken as well as the duration of the overdose.
Evaluate the participant to determine, in consultation with the medical monitor, if possible, whether study intervention should be interrupted

### 6.9: Prior and Concomitant Therapy

Any medication or vaccine (including over-the-counter or prescription medicines, recreational drugs, vitamins, and/or herbal supplements) that the participant is receiving at the time of enrolment or receives during the study must be recorded along with:
Reason for use
Dates of administration including start and end dates
Dosage information including dose and frequency

The medical monitor should be contacted if there are any questions regarding concomitant or prior therapy.

### Prior and Concomitant Medications Review

The Investigator or qualified designee will review prior medication use, and record prior medications taken by the participant within 30 days prior to screening as well as all relevant MG therapies since diagnosis.

The Investigator or qualified designee will record medication, if any, taken by the participant during the study through the last visit. Concomitant medications will be recorded for 14 days after the last dose of study intervention (or longer if related to an SAE).

Patients on treatment of steroids are required to be on a stable dose for 4 weeks prior to screening and the dose should be expected to remain stable for the duration of the study.

Patients on treatment of azathioprine, ciclosporin, methotrexate, mycophenolate mofetil, and tacrolimus (or any other off-label treatment for MG) are required to have been treated for at least 6 months prior to screening and to be on a stable dose for a least 2 months prior to screening and the dose should be expected to remain stable for the duration of the study.

Patients on ACh-esterase inhibitors are required to be on a stable dose for 2 months prior to screening and the dose should be expected to remain stable for the duration of the study. In addition, patients should withhold ACh-esterase inhibitor treatment on the day of testing at the baseline, 1-week treatment visit (day 7), and the end of treatment visit (day 21).

Use of intravenous or subcutaneous immunoglobulin or plasma exchange up to 4 weeks prior to screening are not allowed.

Use of rituximab up to 6 months prior to screening is not allowed.

Use of FcRn-antagonists or C5 inhibitors are not allowed. For Efgartigimod, Rozanolixizumab, Eculizumab and Zilucoplan, the patients should have received the last dose at least 3 months prior to screening. For Ravulizumab the patients should have received the last dose at least 9 months prior to screening.

The following medications are prohibited during the study:
Anticholinergic drugs (e.g., atropine, benztropine, orphenadrine, scopolamine)
Muscle relaxants (e.g., carisoprodol, cyclobenzaprine, diazepam, metaxalone) or benzodizepines.
NOTE: Short-acting benzodiazepines used for night-time treatment of insomnia are permitted if on a stable dose for 2 months prior to screening and expected to remain stable for the duration of the study
Drugs lowering serum uric acid:
   Xanthine oxidase inhibitors (eg. allopurinol, febuxostat, topiroxostat)
   Uricosuric agents (eg. benzbromarone, probenecid, arhalofenate)
Potent or moderately potent inhibitors of OATP1B1, BCRP, OAT1, OAT3, MATE2K, or OAT2 (e.g., probenecid, diclofenac, pyrimethamine)
Sensitive or moderately sensitive substrates of OAT3, BCRP, and URAT1 (e.g., topetecan, rosuvastatin, oseltamivir, olmesartan)

Coronavirus disease 2019 (COVID-19) vaccines should be avoided from 1 week prior to screening to 1 week after the last dose.

Other current and recent (within 1 month prior to the screening) treatments will be allowed, if judged by the Investigator to be of no relevance for the study.

### 7: Discontinuation of Study Intervention and Participant Discontinuation (Including Stopping Rules)

Discontinuation of specific sites or of the study as a whole are handled as part of the appendix on Governance.

Participants who are discontinued prematurely before completing Visit 5 (end of treatment [EOT]) may be replaced.

### 7.1: Discontinuation of Study Intervention

Participants may be discontinued from study intervention in the following situations:
Participant decision. The participant is at any time free to discontinue treatment, without prejudice to further treatment.
An AE that, in the opinion of the Investigator and/or Sponsor, warrants discontinuation from further dosing. E.g., in the event of severe myotonia (i.e., severe stiffness exists, that impairs every duty and activity) the Investigator might consult with the Sponsor to evaluate permanent treatment discontinuation.
The participant experiences suicidal ideation and behaviour (SIB) that warrants discontinuation from further dosing after risk assessment, see Section 8.3.7.
Safety reasons as judged by the Investigator and/or Sponsor where continued treatment may put the participant at undue risk.

Participants will be discontinued from study intervention in the following situations:
The participant becomes pregnant during the course of the study, see Section 8.4.5.
The participant has the following liver chemistry findings:
   Alanine aminotransferase (ALT) or aspartate aminotransferase (AST)>8×ULN
   ALT or AST>5×ULN for more than 2 weeks
   AST or ALT>3×ULN and total bilirubin>2×ULN.
   AST or ALT>3×ULN and international normalised ratio (INR)>1.5.
   AST or ALT>3×ULN with the appearance of fatigue, nausea, vomiting, right upper quadrant pain or tenderness, fever, rash, and/or eosinophilia (>5%).
The participant has the following QTcF findings:
Participant without bundle branch block:
   QTcF>500 msec or change from baseline of QTcF>60 msec
Participant with bundle branch block:
   QTcF>500 msec if baseline value<450 msec
   QTcF>530 msec if baseline value≥450 to 480 msec

If study intervention is permanently discontinued, the participant should, if at all possible, remain in the study to be evaluated for follow-up assessments. See the SoA (FIGS. 19A-19B) for data to be collected at the time of follow-up and for any further evaluations that need to be completed.

7.2: Participant Discontinuation/Withdrawal from the Study

A participant may withdraw from the study at any time at the participant's own request or for any reason (or without providing any reason). A participant may be withdrawn at any time at the discretion of the Investigator for safety, or compliance reasons. The participant will be permanently discontinued from the study intervention and the study at that time.

If the participant withdraws consent for disclosure of future information, the Sponsor may retain and continue to use any data collected before such a withdrawal of consent. If a participant withdraws from the study, the participant may request destruction of any samples taken and not tested, and the Investigator must document this in the site study records.

At the time of discontinuing from the study, if possible, an early discontinuation visit should be conducted, as shown in the SoA (FIGS. 19A-19B). Refer to the SoA for data to be collected at the time of study discontinuation and follow-up and for any further evaluations that need to be completed.

Participation may be terminated prior to completing the study and the reason recorded as follows:
Adverse event
Protocol violation
Loss to follow-up
Participant withdrew consent at own request
Other

### 7.3: Loss to Follow-Up

A participant will be considered lost to follow-up if the participant repeatedly fails to return for scheduled visits and is unable to be contacted by the study site.

The following actions must be taken if a participant fails to return to the study site for a required study visit:
The site must attempt to contact the participant and reschedule the missed visit as soon as possible (and within the visit window, where one is defined), counsel the participant on the importance of maintaining the assigned visit schedule and ascertain whether the participant wishes to and/or should continue in the study.
In cases in which the participant is deemed lost to follow-up, the Investigator or designee must make every effort to regain contact with the participant. Contact attempts should be documented in the participant's medical record/case report form (CRF).

### 8: Study Assessments and Procedures

Study procedures and their order are summarised in the SoA (FIGS. 19A-19B). Protocol waivers or exemptions are not allowed.

Adherence to the study design requirements, including those specified in the SoA, is essential and required for study conduct.

All screening evaluations must be completed and reviewed to confirm that potential participants meet all eligibility criteria. The Investigator will maintain a screening log to record details of all participants screened and to confirm eligibility or record reasons for screening failure, as applicable.

Procedures conducted as part of the participant's routine clinical management (e.g., blood count) and obtained before signing of the ICF may be used for screening or baseline purposes provided the procedures met the protocol-specified criteria and were performed within the timeframe defined in the SoA.

In the event of a significant study-continuity issue (e.g., caused by a pandemic), alternate strategies for participant visits, assessments, medication distribution and monitoring may be implemented by the Sponsor or the Investigator, as per local health authority/ethics requirements.

Safety/laboratory/analyte results that could unblind the study will not be reported to investigative sites or other blinded personnel until the study has been unblinded.

Over the duration of the study, the expected maximum amount of blood collected from each participant, including any extra assessments that may be required, will be much lower than 250 mL over an 8-week period.

Repeat or unscheduled samples may be taken for safety reasons or for technical issues with the samples.

### Participant Identification Card

All participants will be given a participant identification card identifying them as participants in a research study. The card will contain study site contact information (including direct telephone numbers) to be used in the event of an emergency. The Investigator or qualified designee will provide the participant with a participant identification card immediately after the participant provides written informed consent. At the time of intervention allocation/randomisation, site personnel will add the intervention/randomisation number to the participant identification card.

The participant identification card also contains contact information for the emergency unblinding call centre so that a healthcare provider can obtain information about study intervention in emergency situations where the Investigator is not available.

### Calibration of Equipment

The Investigator (or qualified designee) is responsible for ensuring that any device or instrument used for a clinical evaluation/test during the study that provides information about eligibility criteria and/or safety or efficacy parameters is suitably calibrated and/or maintained to ensure that the data obtained are reliable and/or reproducible. Documentation of equipment calibration must be retained as source documentation at the study site.

### 8.1: Administrative and Baseline Procedures

### Medical History

A medical history will be obtained by the Investigator or qualified designee. The medical history will collect all active conditions and any condition diagnosed that the Investigator considers to be clinically relevant. Details regarding the disease for which the participant has enrolled in this study will be recorded separately and not listed as medical history.

### Substance Use/Drug Screen

The use of drugs (of potential abuse) can influence the measurements and the ability to perform on the functional outcome measures of the study. Therefore, a drug test, including cocaine, heroin, opiates, and marijuana will be performed at screening. Participants with positive drug screen (cocaine, heroin, opiates) or marijuana drug abuse (as determined by positive drug screen and judged by the investigator) will not be allowed in the study (as defined in exclusion criterion 10).

### 8.2: Efficacy Assessments

The efficacy assessments planned at each visit are provided in the SoA (FIGS. 19A-19B). To the extent possible the efficacy assessments should be performed in the order outlined in the SoA. On day 7 and day 21, the primary efficacy assessments should be performed 2 hours after administering the afternoon dose.

Clinical assessments will be performed by a trained evaluator at the site (the Investigator or a physical therapist). For each test, each participant should be assessed by the same evaluator for all sessions. The use of assistive devices such as ankle foot orthoses may or may not be permitted during assessments but should be the same for all sessions.

Before and after each clinical assessment, the assessors should ensure that participants are sufficiently rested, including at least 10 minutes of rest after each clinical assessment.

### 8.2.1: Primary Efficacy Assessment

The QMG score for disease severity is a validated clinician rated clinical measure of sentinel muscle groups developed by the MGFA and the gold standard recommended for prospective studies in MG by the MGFA task force. (Jaretzki et al Ann Thorac Surg. 2000 July; 70(1):327-34; Bedlack et al Neurology. 2005 Jun. 14; 64(11):1968-70). The QMG scoring for disease severity will be performed as scheduled in the SoA (FIGS. 19A-19B).

Further details regarding the methodology will be provided in the Assessment Manual. Calculation of derived variables will be defined in the statistical analysis plan (SAP).

### 8.2.2: Other Clinician-Rated Efficacy Outcomes

### 8.2.2.1: Myasthenia Gravis Composite (MGC)

The MGC assesses disease specific symptoms and examination findings derived from the MG-ADL (patient reported), QMG and Manual Muscle Test (MMT) (clinician determined). It consists of 10 weighted items. The MG-ADL and QMG items will be derived from the separate MG-ADL and QMG tests. Manual muscle test items include neck flexion or extension (weakest muscle), shoulder abduction, and hip flexion.

### 8.2.3: Patient-Reported Outcomes

Participants will be asked to complete 3 questionnaires: MG-ADL, MG-QOL15r and Neuro-QoL Fatigue Short Form.

Participants will be given the questionnaires as paper forms to complete. They will be allowed to use a proxy if they have physical limitations preventing them from filling out the questionnaires.

8.2.3.1: Myasthenia Gravis Activities of Daily Living response (MG-ADL)

MG-ADL, an 8-item patient-reported outcome measure that assesses MG specific symptoms and their impact on daily activities, will be performed as scheduled in the SoA (FIGS. 19A-19B).

Further details regarding the methodology will be provided in the Assessment Manual.

### 8.2.3.2: Myasthenia Gravis Quality of Life15-Item Scale Revised (MG-QoL15r)

The MG-QOL15r is a 15-item patient-reported outcome measure assessing physical, psychological and social domains commonly impacted by MG.

### 8.2.3.3: Neuro-QoL Fatigue Short Form

The Neuro-QoL Fatigue Short Form is a patient reported outcome that assesses fatiguability on 8 items on a 5-point scale.

### 8.3: Safety Assessments

The safety assessments planned at each visit are provided in the SoA (FIGS. 19A-19B).

### 8.3.1: Height and Weight

Height and weight will be measured and recorded.

### 8.3.2: Physical Examinations

A complete physical examination will include, at a minimum, assessments of the neurological, cardiovascular, respiratory, gastrointestinal, musculoskeletal, and skin systems.
A brief physical examination will include, at a minimum, assessments of the neurological and musculoskeletal systems.

Investigators should pay special attention to clinical signs related to previous serious illnesses.

### 8.3.3: Vital Signs

Vital signs will be measured in a supine position after 5 minutes rest and will include tympanic temperature, systolic and diastolic blood pressure, and pulse and respiratory rate.

Three readings of blood pressure and pulse will be taken at least 1 minute apart.

For orthostatic vital signs measurements, participants will be supine for at least 5 minutes before obtaining a single measurement of blood pressure and pulse. Participants will then stand for at least 2 minutes before obtaining a single measurement of blood pressure and pulse.

### 8.3.4: Electrocardiograms (ECGs)

Triplicate 12-lead ECG(s) will be obtained as outlined in the SoA (FIGS. 19A-19B) using an ECG machine that automatically calculates the heart rate and measures PR, QRS, QT, RR, and QTcF intervals.

For triplicate ECG recordings, 3 individual ECG tracings should be obtained as closely as possible in succession, but no more than 2 minutes apart. The average of the triplicate measures should be used for assessing ECG parameters.

For participants showing an increase of QTcF by >60 msec (regardless of the baseline value) or abnormalities in QTcF (as defined in exclusion criterion 5), persisting for >5 minutes, further evaluation is recommended, including repeated ECGs. A cardiologist may be consulted. Refer to Section 7.1 for QTcF withdrawal criteria.

The central ECG review vendor, as the responsible company for the centralised ECG assessments, will provide the study sites with standardised ECG equipment and supplies, specific training and written instructions. Following an acquisition of a quality ECG tracing, the Investigator or designee will electronically transfer the data to the central ECG review vendor.

Investigators will assess the eligibility of the participant according to the ECG report of Visit 1 from the central reader. Eligibility regarding exclusion criterion 5 will be determined by the central reader. Any abnormal finding in the ECG tracing will also be evaluated by the Investigator or by a hospital cardiologist during the visit and will be specifically documented and registered in the CRF. Throughout the study, clinically relevant new findings or worsening of a pre-existing finding in the ECGs (parameters or abnormal findings in the tracing) must be considered an AE and must be recorded in the AE CRF form.

### 8.3.5: Clinical Safety Laboratory Tests

Refer to Appendix 2 for the list of clinical laboratory tests to be performed and the SoA (FIGS. 19A-19B) for the timing and frequency.

The Investigator must review the laboratory results, document this review, and record any clinically significant changes occurring during the study as an AE. The laboratory results must be retained with source documents.

Abnormal laboratory findings associated with the underlying disease are not considered clinically significant unless judged by the Investigator to be more severe than expected for the participant's condition.

All laboratory tests with values considered clinically abnormal during participation in the study or within 14 days after the last dose of study intervention should be repeated until the values return to normal or baseline or are no longer considered clinically significant by the Investigator or Medical Monitor.
If clinically significant values do not return to normal/baseline within a period of time judged reasonable by the Investigator, the aetiology should be identified, where possible, and the Sponsor notified.
All protocol-required laboratory tests, as defined in Appendix 2, must be conducted in accordance with the Laboratory Manual and the SoA (FIGS. 19A-19B)
If laboratory values from laboratory tests not specified in the protocol and performed at the institution's local laboratory result in the need for a change in participant management or are considered clinically relevant by the Investigator (e.g., are considered to be an SAE or an AE or require dose modification), then the results must be recorded.

In the Phase 1 clinical study, a dose-dependent decrease in serum uric acid was observed, which was considered non-clinically significant. Due to the dose-dependency of the decrease in serum uric acid, reporting of the serum uric acid values during trial conduct would be unblinding. Therefore, all serum uric acid values will be reported at the end of the trial. Since the decrease in serum uric acid is generally considered non-clinically significant, it is not reported as AEs during the study. In order to ensure patient safety throughout the trial the medical monitor will review uric acid values in a blinded fashion.

### 8.3.6: Pregnancy Testing

Refer to Inclusion Criteria 10 for pregnancy testing entry criteria.
Serum or urine pregnancy testing should be conducted as specified in the SoA (FIGS. 19A-19B).
Additional serum or urine pregnancy tests may be performed, as determined necessary by the Investigator or required by local regulation, to establish the absence of pregnancy at any time during participation in the study.

### 8.3.7: Suicidal Ideation and Behaviour Risk Monitoring

SIB assessments should be conducted whether or not a particular product is known or suspected to be associated with treatment-emergent suicidal ideation and behavior. Participants being treated with NMD670 should be monitored appropriately and observed closely for SIB or any other unusual changes in behaviour, especially at the beginning and end of the course of intervention.

Participants will be monitored for SIB throughout the study, using C-SSRS, as specified in the SoA (FIGS. 19A-19B).

Participants who experience signs of SIB should undergo a risk assessment. All factors contributing to SIB should be evaluated and consideration should be given to discontinuation of the study intervention.

### 8.4: Adverse Events (AEs), Serious Adverse Events (SAEs), and Other Safety Reporting

The definitions of AEs and SAEs can be found in Appendix 3.

The Investigator and any qualified designees are responsible for detecting, documenting, and reporting events that meet the definition of an AE or SAE and remain responsible for following up all AEs (see Section 7). This includes events reported by the participant (or, when appropriate, by a caregiver, surrogate, or the participant's legally authorised representative).

The method of recording, evaluating, and assessing causality of AEs and SAEs and the procedures for completing and transmitting SAE reports are provided in Appendix 3.

### 8.4.1: Time Period and Frequency for Collecting AE and SAE Information

All AEs and SAEs observed by the investigator or participant, must be reported by the investigator and evaluated. All events meeting the definition of an AE must be collected and reported. This includes events from the signing of ICF until the follow-up visit, as specified in the SoA (FIGS. 19A-19B).

All SAEs will be recorded and reported to the Sponsor or designee within 24 hours of awareness, as indicated in Appendix 3. The Investigator will submit any updated SAE data to the Sponsor within 24 hours of it being available.

Investigators are not obliged to actively seek information on AEs or SAEs after the conclusion of study participation. However, if the Investigator learns of any SAE, including a death, at any time after a participant has been discharged from the study, and the Investigator considers the event to be reasonably related to the study intervention or study participation, the Investigator must promptly notify the Sponsor.

### 8.4.2: Method of Detecting AEs and SAEs

Care will be taken not to introduce bias when detecting AEs and/or SAEs. Open-ended and non leading verbal questioning of the participant is the preferred method to inguire about AE occurrence.

### 8.4.3: Follow-Up of AEs and SAEs

After the initial AE/SAE report, the Investigator is required to proactively follow each participant at subsequent visits/contacts. All SAEs will be followed until resolution, stabilisation, until the event is otherwise explained, or the participant is lost to follow-up (as defined in Section 7.3). Further information on follow-up procedures is given in Appendix 3.

### 8.4.4: Regulatory Reporting Requirements for SAE

Prompt notification (within 24 hours, see Appendix 3) by the Investigator to the Sponsor (or designee) of an SAE is essential so that legal obligations and ethical responsibilities towards the safety of participants and the safety of a study intervention under clinical investigation are met.

The Sponsor has a legal responsibility to notify both the local regulatory authority and other regulatory agencies about the safety of a study intervention under clinical investigation. The Sponsor will comply with country-specific regulatory requirements relating to safety reporting to the regulatory authority, IRB (institutional review board)/IEC (independent ethics committee), and Investigators.

An Investigator who receives an Investigator Safety Report describing an SAE or other specific safety information (e.g., summary or listing of SAEs) from the Sponsor will review and then file it along with the Investigator's Brochure and will notify the IRB/IEC, if appropriate according to local requirements.

All SAEs that occur during the study, and all SAEs occurring up to the follow-up visit 6 days after receiving the last dose of study intervention, whether considered to be associated with the study intervention or not, must be reported within 24 hours to the Sponsor (or designee).

Investigator safety reports must be prepared for suspected unexpected serious adverse reactions (SUSARs) according to local regulatory requirements and Sponsor policy and forwarded to Investigators as necessary.

Regarding SAEs after the conclusion of study participation, refer to Section 8.4.1.

### 8.4.5: Pregnancy

Details of all pregnancies in female participants and partners of male participants will be collected after the start of study intervention and until 14 days after the last dose of study intervention.

If a pregnancy is reported, the Investigator will record pregnancy information on the appropriate form and submit it to the Sponsor (or designee) within 24 hours of learning of the female participants or partner of male participant (after obtaining the necessary signed informed consent from the partner) pregnancy, as detailed in Section 10.3.4.

While pregnancy itself is not considered to be an AE or SAE, any pregnancy complication or elective termination of a pregnancy for medical reasons will be reported as an AE or SAE.

Abnormal pregnancy outcomes (e.g., maternal serious complications, therapeutic abortion, spontaneous abortion, foetal death, stillbirth, congenital anomalies, ectopic pregnancy) are considered to be SAEs and will be reported as such.

The participant/pregnant partner will be followed to determine the outcome of the pregnancy. The Investigator will collect follow-up information on the participant/pregnant partner and the neonate and the information will be forwarded to the Sponsor.

Any post-study pregnancy-related SAE considered reasonably related to the study intervention by the Investigator will be reported to the Sponsor as described in Section 8.4.4. While the Investigator is not obligated to actively seek this information in former study participants/pregnant partners, he or she may learn of an SAE through spontaneous reporting.

Any female participant who becomes pregnant while participating in the study will discontinue study intervention.

### 8.5: Pharmacokinetics

A single whole blood sample of approximately 3 mL will be collected for measurement of plasma concentrations of NMD670 and its metabolite (NMD1190) will be taken at the timepoints specified in the SoA (FIGS. 19A-19B). The timing of sampling may be altered during the course of the study based on newly available data (e.g., to obtain data closer to the time of peak plasma concentrations) to ensure appropriate monitoring. Instructions for the collection and handling of biological samples will be provided by the Sponsor. The actual date and time (24 hour clock time) of each sample collection will be recorded.

Blood sample collection, processing and shipping details will be outlined in a separate Laboratory Manual. In brief, blood will be processed and plasma analysed using validated assays.

Intervention concentration information that would unblind the study will not be reported to investigative sites or blinded personnel until the study has been unblinded.

### 8.6: Genetics

Genetics are not evaluated in this study.

### 8.7: Biomarkers

Biomarkers are not evaluated in this study.

### 8.8: Immunogenicity Assessments

Immunogenicity is not assessed during the study.

### 8.9: Storage and Future Use of Biological Samples

NMD Pharma A/S will be responsible for ensuring all biological sample collection and storage is secure and with adequate measures to protect confidentiality, per all applicable local regulations.

All biological samples will be destroyed after analyses; no future use of biological samples is planned for this study.

### 8.10: Medical Resource Utilisation and Health Economics

Medical resource utilisation and health economics parameters are not evaluated in this study.

### 9: Statistical Considerations

To overcome the shortcomings of traditional dose selection methods, the dose response will be investigated using MCP-Mod 9. In 2014, EMA issued a Qualification Opinion that MCP-Mod is an efficient statistical methodology for model-based design and analysis of Phase 2 dose finding studies under model uncertainty. In 2016, FDA granted the MCP-Mod fit-for-purpose designation as an adequate and appropriate method for guiding dose selection for phase 3 testing.
The MCP-Mod method will include treatment group, baseline scores, and stratification based on autoantibody status (MuSK versus AChR) and disease severity (patients with QMG_17).

The SAP will be finalised prior to unblinding and database lock. It will include a more technical and detailed description of the statistical analyses described in this section. This section is a summary of the planned statistical analyses of the most important endpoints, including primary and secondary endpoints.

The primary efficacy endpoint will be analysed in all randomly assigned patients, by means of the treatment assigned for analysis (intention-to-treat principle). Safety outcomes will be analysed in all randomly assigned patients who received at least one dose of treatment according to treatment actually received (Safety Set).

MCPMod will be used to analyse the dose-response relationship and determine the dose-response shape of NMD670 by testing if there is a non-flat dose-response relationship between the different doses of NMD670 and placebo. The model will include treatment group, baseline scores, and stratification based on autoantibody status (MuSK versus AchR) and disease severity (moderate versus severe).

### 9.1: Statistical Hypotheses

The primary clinical question of interest for the objective of clinical efficacy as rated by patients is:
Is there a non-flat exposure-response pattern in change from baseline to day 21 of QMG total score in adult patients with moderate to severe Myasthenia Gravis treated with placebo versus NMD670?

The null hypothesis is that there is a flat exposure-response pattern across placebo and increasing doses of NMD670. The alternative hypothesis is that there is a non-flat exposure-response pattern indicating a benefit of NMD670 compared with placebo.

### 9.1.1: Multiplicity Adjustment

The test of dose-response using MCP-Mod with a single model, namely, an Emax model is a single hypothesis test thus requiring no adjustment for multiplicity. If the test of dose-response is statistically significant then hypothesis testing will continue by comparing individual dose levels of NMD670 with placebo without adjustment for multiplicity for these Secondary Endpoints.

### 9.2: Analysis Sets

TABLE 22
Participant Analysis Sets

| Participant Analysis | |
|---|---|
| Set | Description |
| Full analysis set | All randomised participants who received at |
| (FAS) | least one dose of study intervention. |
| Efficacy analysis | Participants in the FAS without major protocol |
| set (EAS) | violations that may affect the efficacy analysis. |
| PK analysis set | All randomised participants with at least one |
| | quantifiable PK concentration for NMD670 or |
| | its metabolite NMD1190. |

Abbreviations:
EAS = efficacy analysis set;
FAS = full analysis set;
PK = pharmacokinetic(s)

The FAS will be used to analyse endpoints related to the efficacy and safety. The EAS will be used for sensitivity analysis of endpoints and assessments related to efficacy. The PK data will be summarised using the PK analysis set.

For the efficacy analyses, participants will be included in the analyses according to the planned study intervention; whereas for safety analyses, participants will be included in the analyses according to the study intervention they actually received in the treatment period.

Additional analysis sets may be defined to support specific analyses and will be documented in the SAP prior to unblinding.

### 9.3: Statistical Analyses

### 9.3.1: General Considerations

Listings will be generated for all endpoints.

Summary tables and analysis for safety efficacy, PK, and PD data will be presented by treatment. The main comparison of interest will always be NMD670 vs placebo.

Unless stated otherwise, mean, standard deviation, median, first (Q1) and third (Q3) quartiles, minimum and maximum will be presented when summarising continuous variables. Counts and frequencies will be used for categorical variables.

For safety, efficacy, and PD measurements, baseline will be the last pre-dose value.

Statistical hypotheses will be tested at a two-sided significance level of 5%. All confidence intervals will have a 95% level of confidence unless stated otherwise.

An overview of study disposition will be provided, including participants randomised and treated with NMD670 and placebo at each site. Reasons for withdrawals will be summarised by primary reason.

### 9.3.2: Primary Endpoint Analysis

### 9.3.2.1: Definition of Estimand

The primary clinical question of interest for the objective of clinical efficacy as rated by the clinician is:
Is there a non-flat exposure-response pattern in change from baseline to day 21 of QMG total score in adult patients with Myasthenia Gravis treated with placebo versus NMD670?

Specifically, the estimand has the following attributes:
Population:
   The target population of interest are male and female patients aged 18 to 75 years (both included), diagnosed with MG defined as MGFA class II-IV, a QMG score of 11 or more and an MG-ADL score of 6 or more at screening. These are all defined by the inclusion and exclusion criteria
Patient-Level Endpoint:
   The change from baseline to day 21 in QMG score will be derived for each patient. The baseline value will be the QMG score closest (but before) first dose
Intercurrent Events
   Anticipated Intercurrent Events include treatment discontinuation and treatment noncompliance. The treatment policy strategy will be used with respect to these types of events; thus, all available data will be used
Population Level Summary
   Point estimates (and confidence intervals) of the parameters from the dose-response model will be used to characterise the dose-response relationship. Placebo will be considered as dose 0 in this model
   The primary efficacy endpoint will be analysed in all randomly assigned patients, by means of the treatment assigned for analysis (Full Analysis Set).

### 9.3.2.2: Analytical Method

### Main Analytic Approach

A 4-parameter Emax model is proposed as the only candidate model to fit within the MCPMod framework on the basis that other models (e.g., linear, log-linear, quadratic, 3-parameter Emax) are unlikely to be consistent with the expected dose-response and thus avoiding a penalty with alpha adjustment. The Emax model will be fit using the DoseFinding R package (or equivalent). Covariates will be included in the multiple comparisons test using the addCovars option in the DoseFinding R package (or equivalent). Point estimates and 95% confidence intervals of model parameters will be reported.

Missing data for the primary endpoint will be imputed using a multiple imputation approach (the details of which will be included in the SAP which will be finalised before unblinding).

### 9.3.3: Secondary Endpoints Analysis

Pairwise comparisons (without adjustment for multiplicity) of individual dose levels versus placebo, (e.g., high dose level versus placebo) will be performed for all key secondary and secondary endpoints.

### 9.3.4: Safety Analyses

Safety outcomes will be analysed in all randomly assigned patients who received at least one dose of treatment according to treatment actually received (Safety Set). Safety endpoints will be evaluated using descriptive statistics.

Further details will be provided in the SAP which will be finalised prior to unblinding.

### 9.3.5: Other Analyses

Pharmacokinetic parameters and/or plasma concentrations will be listed.

Other exploratory endpoints will be summarised descriptively.

Exploratory analyses will be detailed in the SAP.

### 9.4: Interim Analyses

No interim analysis is planned.

### 9.5: Sample Size Determination

The sample size calculation is based on change from baseline to day 21 in the Quantitative Myasthenia Gravis Score. Based on an aggregate literature review of the treatment effect of other products used in the management of MG. The placebo subjects mean and standard deviation at day 21 are −1.7 and 6.2 respectively.

For the purpose of establishing a dose-response based on change from baseline to day 21 in the Quantitative Myasthenia Gravis Score, it is assumed that a 4-parameter Emax model will adequately describe the dose-response curves:
ED50=500 mg (total daily dose)
Gamma (Hill)=1.2823 (consistent with PK models)
E0=−1.7 (mean Quantitative Myasthenia Gravis Score change from baseline on Placebo)
Emax (at asymptote)=−6.9 (i.e., anticipated mean difference from placebo=−5.2) Assuming an allocation of patients on a 1:1:1:1 ratio, a two-sided alpha of 5%, and a standard deviation of 6.2 (based on historical data) (Howard et al Lancet Neurol. 2017 December; 16(12):976-986; Neurol Ther. 2023 October; 12(5):1435-1438; Vu et al NEJM Evid. 2022 1(5):1-12; Howard et al JAMA Neurol. 2020 May 1; 77(5):582-592; Bril et al Neurology. 2021 Feb. 9; 96(6):e853-e865), it is estimated that a total of 80 participants (20:20:20:20 per group, respectively) will have 80% power to establish a statistically significant dose-response.

Approximately 110 participants will be screened (assuming a screen failure rate of ˜25% based on historical data) to achieve approximately 80 randomised participants (Howard et al Lancet Neurol. 2021 July; 20(7):526-536; Howard et al Lancet Neurol. 2017 December; 16(12):976-986; Vu et al NEJM Evid. 2022 1(5):1-12; Bril et al Neurology. 2021 Feb. 9; 96(6):e853-e865).

### 10.2: Appendix 2

The tests detailed in Table 23 will be performed by the central laboratory (exception is local urine pregnancy test as outlined in footnote 3 to Table 23).

Local laboratory results are only required in the event that the central laboratory results are not available in time for either confirmation of eligibility, study intervention administration and/or response evaluation. If a local sample is required, it is important that the sample for central analysis is obtained at the same time. Additionally, if the local laboratory results are used to make either a study intervention decision or response evaluation, the results must be recorded.

Protocol-specific requirements for inclusion or exclusion of participants are detailed in Section 5 of the protocol.

Additional tests may be performed at any time during the study as determined necessary by the Investigator or required by local regulations.

Investigators must document their review of each laboratory safety report.

TABLE 23
Protocol-required Safety Laboratory Tests

| Laboratory | | |
|---|---|---|
| Tests | Parameters | |
| Haematology | Platelet count | WBC count with |
| | RBC count | differential: |
| | Haemoglobin | Neutrophils |
| | Haematocrit | Lymphocytes |
| | RBC indices: | Monocytes |
| | MCV | Eosinophils |
| | MCH | Basophils |
| | MCHC | |
| Haemostasis | INR | aPTT |
| | Prothrombin time | Fibrinogen |
| Clinical | BUN | AST |
| chemistry ⁰ | Potassium | Total and direct |
| | Creatinine | bilirubin |
| | Sodium | ALT |
| | Glucose (after at | GGT |
| | least 4-hour fasting) | Total protein |
| | Calcium | Albumin |
| | Inorganic phosphate | Triglycerides |
| | Creatine kinase | LDH |
| | Chloride | Alkaline phosphatase ⁰ |
| | Bicarbonate | Uric acid |

| | |
|---|---|
| Routine | Specific gravity |
| urinalysis | Uric acid, Creatinine and Uric Acid to |
| | Creatinine ratio pH, glucose, protein, blood, |
| | ketones, bilirubin, urobilinogen, nitrite, |
| | leukocyte esterase by dipstick |
| | Microscopic examination (if blood or protein |
| | is abnormal) |
| Pregnancy | Highly sensitive serum or urine hCG pregnancy |
| testing | test (as needed for women of childbearing |
| | potential) at timepoints detailed in the |
| | SoA (FIGS. 19A-19B).⁰ |
| Other | Follicle-stimulating hormone and oestradiol |
| screening | (as needed in women of non-childbearing |
| tests | potential only) |
| | Urine drug screen (to include cocaine, heroin, |
| | opiates, and marijuana) ⁰ |
| | Serology (HIV antibody, HBsAg, and hepatitis C antibody |

NOTES:
Details of liver chemistry follow-up are given in Section Error! Reference source not found . . .
If alkaline phosphatase is elevated, consider fractionating.
After screening, local urine testing will be standard for the protocol, unless serum testing is required by local regulation or IRB/IEC, or in order to confirm positive or inconclusive urine test.
Refer to Exclusion Criterion Error! Reference source not found. (Section Error! Reference source not found.) for details.
Abbreviations: ALT = alanine aminotransferase; aPTT = activated partial thromboplastin time; AST = aspartate aminotransferase; BUN = blood urea nitrogen; GGT = gamma-glutamyl transferase; HBsAg = hepatitis B surface antigen; hCG = human chorionic gonadotropin; HIV = human immunodeficiency virus; IEC = independent ethics committee; INR = international normalised ration; IRB = institutional review board; LDH = lactate dehydrogenase; MCH = mean corpuscular haemoglobin; MCHC = mean corpuscular haemoglobin concentration; MCV = mean corpuscular volume; RBC = red blood cell; ULN = upper limit of normal; WBC = white blood cell.

### 10.3: Appendix 3: AEs and SAEs: Definitions and Procedures for Recording, Evaluating, Follow-Up, and Reporting

### 10.3.1: Definition of AE

### AE Definition

An AE is any untoward medical occurrence in a patient or clinical study participant, temporally associated with the use of a study intervention, whether or not considered related to the study intervention.

NOTE: An AE can therefore be any unfavourable and unintended sign (including an abnormal laboratory finding), symptom, or disease (new or exacerbated) temporally associated with the use of a study intervention, whether or not considered related to the study intervention.

### Events Meeting the AE Definition

Any abnormal laboratory test results (haematology, clinical chemistry, or urinalysis) or other safety assessments (e.g., ECG, radiological scans, vital signs measurements), including those that worsen from baseline, considered clinically significant in the medical and scientific judgement of the Investigator (e.g., not related to progression of underlying disease, or more severe than expected for the participant's condition).

Exacerbation of a chronic or intermittent pre-existing condition including either an increase in frequency and/or intensity of the condition.

New condition detected or diagnosed after study intervention administration even though it may have been present before the start of the study.

Signs, symptoms, or the clinical sequelae of a suspected intervention-intervention interaction.

Signs, symptoms, or the clinical sequelae of a suspected overdose of either study intervention or a concomitant medication. Overdose per se will not be reported as an AE/SAE unless it is an intentional overdose taken with possible suicidal/self-harming intent. Such overdoses should be reported regardless of sequelae.

Lack of efficacy or failure of expected pharmacological action per se will not be reported as an AE or SAE. Such instances will be captured in the efficacy assessments. However, the signs, symptoms, and/or clinical sequelae resulting from lack of efficacy will be reported as AE or SAE if they fulfil the definition of an AE or SAE.

### Events NOT Meeting the AE Definition

Any abnormal laboratory findings or other abnormal safety assessments that are associated with the underlying disease, unless judged by the Investigator to be more severe than expected for the participant's condition.

The disease/disorder being studied or expected progression, signs, or symptoms of the disease/disorder being studied, unless more severe than expected for the participant's condition.

Medical or surgical procedure (e.g., endoscopy, appendectomy): the condition that leads to the procedure is the AE.

Situations in which an untoward medical occurrence did not occur (social and/or convenience admission to a hospital).

Anticipated day-to-day fluctuations of pre-existing disease(s) or condition(s) present or detected at the start of the study that do not worsen.

### 10.3.2: Definition of SAE

An SAE is an AE that:
a. Results in death
It should be noted that death per se is not an AE, rather an outcome. The Investigator should always try to report an AE that was considered as immediate cause of death.
b. Is life-threatening
The term life-threatening in the definition of serious refers to an event in which the participant was at risk of death at the time of the event. It does not refer to an event, which hypothetically might have caused death, if it were more severe.
c. Requires inpatient hospitalisation or prolongation of existing hospitalisation
In general, hospitalisation signifies that the participant has been admitted (usually involving at least an overnight stay) at the hospital or emergency ward for observation and/or intervention that would not have been appropriate in the physician's office or outpatient setting. Complications that occur during hospitalisation are AE. If a complication prolongs hospitalisation or fulfils any other serious criteria, the event is serious. When in doubt as to whether hospitalisation occurred or was necessary, the AE should be considered serious.
Hospitalisation for elective intervention of a pre-existing condition that did not worsen from baseline is not considered an AE.
d. Results in persistent or significant disability/incapacity
The term disability means a substantial disruption of a person's ability to conduct normal life functions.
This definition is not intended to include experiences of relatively minor medical significance such as uncomplicated headache, nausea, vomiting, diarrhoea, influenza, and accidental trauma (e.g., sprained ankle) that may interfere with or prevent everyday life functions but do not constitute a substantial disruption.
e. Is a congenital anomaly/birth defect
f. Other situations
Medical or scientific judgement should be exercised by the Investigator in deciding whether SAE reporting is appropriate in other situations such as significant medical events that may jeopardise the participant or may require medical or surgical intervention to prevent one of the other outcomes listed in the above definition. These events should usually be considered serious.
Examples of such events are intensive treatment in an emergency room or at home for allergic bronchospasm; blood dyscrasias or convulsions that do not result in hospitalisation; or development of drug dependency or drug abuse.

### 10.3.3: Recording and Follow-Up of AE and SAE

### AE and SAE Recording

AEs should be collected and recorded for each participant from the date the ICF is signed until the end of their participation in the study, i.e., the participant has discontinued or completed the study.

When an AE/SAE occurs, it is the responsibility of the Investigator to review all documentation (e.g., hospital progress notes, laboratory, and diagnostics reports) related to the event.

The Investigator will then record all relevant AE/SAE information.

AEs may be volunteered spontaneously by the participant, or discovered by the study staff during physical examinations or by asking an open, non-leading question such as ‘How have you been feeling since you were last asked?’ All AEs and any required remedial action will be recorded. The nature of AE, date (and time, if known) of AE onset, date (and time, if known) of AE outcome to date, severity and action taken of the AE will be documented together with the Investigator's assessment of the seriousness of the AE and causal relationship to study drug and/or study procedure.

All AEs should be recorded individually in the participant's own words (verbatim) unless, in the opinion of the Investigator, the AEs constitute components of a recognized condition, disease or syndrome. In the latter case, the condition, disease, or syndrome should be named rather than each individual symptom. The AEs will subsequently be coded using the Medical Dictionary for Regulatory Activities (MedDRA).

It is not acceptable for the Investigator to send photocopies of the participant's medical records to the Sponsor (or designee) in lieu of completion of the required form.

There may be instances when copies of medical records for certain cases are requested by the Sponsor (or designee). In this case, all participant identifiers, with the exception of the participant number, will be blinded on the copies of the medical records before submission to the Sponsor (or designee).

The Investigator will attempt to establish a diagnosis of the event based on signs, symptoms, and/or other clinical information. In such cases, the diagnosis (not the individual signs/symptoms) will be documented as the AE/SAE.

### Assessment of Intensity

The Investigator will make an assessment of intensity for each AE and SAE reported during the study and assign a grade according to National Cancer Institute Common Terminology Criteria for AEs [NCI-CTCAE]). The CTCAE displays Grades 1 through 5 with unique clinical descriptions of severity for each AE based on this general guideline:
Grade 1: Mild; asymptomatic or mild symptoms; clinical or diagnostic observations only; intervention not indicated.
Grade 2: Moderate; minimal, local or non invasive intervention indicated; limiting age appropriate instrumental activities of daily living (e.g., preparing meals, shopping for groceries or clothes, using the telephone, managing money).
Grade 3: Severe or medically significant but not immediately life-threatening; hospitalisation or prolongation of hospitalisation indicated; disabling; limiting self care activities of daily living (e.g., bathing, dressing and undressing, feeding self, using the toilet, taking medications, and not bedridden).
Grade 4: Life-threatening consequences; urgent intervention indicated.
Grade 5: Death related to AE (not appropriate for some AEs).
Not all grades are appropriate for all AEs. Therefore, some AEs are listed with fewer than 5 options for grade selection.

An event is defined as ‘serious’ when it meets at least one of the predefined outcomes as described in the definition of an SAE, independently of NCI CTCAE grading.

When changes in the intensity of an AE occur more frequently than once a day, the maximum intensity for the event should be noted for that day. Any change in severity of signs and symptoms over a number of days will be captured by recording a new AE, with the amended severity grade, and the date (and time, if known) of the change.

### Assessment of Causality

The Investigator is obligated to assess the relationship between study intervention and each occurrence of each AE/SAE. The Investigator will use clinical judgement to determine the relationship.

A reasonable possibility of a relationship conveys that there are facts, evidence, and/or arguments to suggest a causal relationship, rather than a relationship cannot be ruled out.

Alternative causes, such as underlying disease(s), concomitant therapy, and other risk factors, as well as the temporal relationship of the event to study intervention administration will be considered and investigated.

The Investigator will also consult the Investigator's Brochure in their assessment.

For each AE/SAE, the Investigator must document in the medical notes that he/she has reviewed the AE/SAE and has provided an assessment of causality.

There may be situations in which an SAE has occurred, and the Investigator has minimal information to include in the initial report in the electronic data collection tool. However, it is very important that the Investigator always make an assessment of causality for every event before the initial transmission of the SAE data to the electronic data collection tool.

The Investigator may change their opinion of causality in light of follow-up information and send an SAE follow-up report with the updated causality assessment.

The following “binary” decision choice will be used by the Investigator to describe the initial causality assessment:
Related: Reasonable possibility of a relatedness
Not related: No reasonable possibility of relatedness.

The causality assessment is one of the criteria used when determining regulatory reporting requirements.

### Follow-Up of AE and SAE

The Investigator is obligated to perform or arrange for the conduct of supplemental measurements and/or evaluations as medically indicated or as requested by the Sponsor (or designee) to elucidate the nature and/or causality of the AE or SAE as fully as possible. This may include additional laboratory tests or investigations, histopathological examinations, or consultation with other health care professionals.

If a participant dies during participation in the study or during a recognised follow-up period, the Investigator will provide the Sponsor (or designee) with a copy of any post-mortem findings including histopathology.

New or updated information will be recorded in the originally completed form.

The Investigator will submit any updated SAE data to the Sponsor within 24 hours of receipt of the information.

### Example 15: Phase 2b Clinical Trial

This example describes a Phase 2b, randomised, double-blind, placebo-controlled study to evaluate the efficacy, safety, and tolerability of 3 dose levels of NMD670 in adult patients with AChR/MuSK-Ab+MG. The study protocol would be similar to that in Example 14, but the participants will be randomised in a 1:1:1:1 ratio to receive one of the following treatments:
Placebo BID
NMD670 100 mg BID
NMD670 200 mg BID
NMD670 400 mg BID

### Example 16: Phase 2b Clinical Trial

This example describes a Phase 2b, randomised, double-blind, placebo-controlled study to evaluate the efficacy, safety, and tolerability of 3 dose levels of NMD670 in adult patients with AChR/MuSK-Ab+MG. The study protocol would be similar to that in Example 14, but the participants will be randomised in a 1:1:1:1 ratio to receive one of the following treatments:
Placebo BID
NMD670 200 mg BID
NMD670 300 mg BID
NMD670 400 mg BID

### Example 17: Phase 2b Clinical Trial

This example describes a Phase 2b, randomised, double-blind, placebo-controlled study to evaluate the efficacy, safety, and tolerability of 3 dose levels of NMD670 in adult patients with AChR/MuSK-Ab+MG. The study protocol would be similar to that in Example 14, but the participants will be randomised in a 1:1:1 ratio to receive one of the following treatments:
Placebo BID
NMD670 200 mg BID
NMD670 400 mg BID

### Example 18: Phase 2b Clinical Trial

This example describes a Phase 2b, randomised, double-blind, placebo-controlled study to evaluate the efficacy, safety, and tolerability of 3 dose levels of NMD670 in adult patients with AChR/MuSK-Ab+MG. The study protocol would be similar to that in Example 14, but the participants will be randomised in a 1:1:1:1 ratio to receive one of the following treatments:
Placebo BID
NMD670 150 mg BID
NMD670 250 mg BID
NMD670 400 mg BID

### REFERENCES

Borges, L. S. and Richman, D. P., Muscle-Specific Kinase, Myasthenia Gravis, Front. Immunol. 2020, 11:707.
Pedersen, T. H., Macdonald, W. A., Broch-Lips, M., Halldorsdottir, O. and Bekgaard Nielsen, O., Chloride channel inhibition improves neuromuscular function under conditions mimicking neuromuscular disorders, Acta Physiol. 2021, 233(2):1-14.
Barnett, C., Herbelin, L., Dimachkie, M. M. and Barohn, R. J., Measuring Clinical Treatment Response in Myasthenia Gravis, Neurol. Clin. 2018, 36(2):339-353.
Barohn, R. J., McIntire, D., Herbelin, L., Wolfe, G. I., Nations, S. and Byran, W. W., Reliability testing of the quantitative myasthenia gravis score, Ann. N. Y. Acad. Sci., 1998, 841:769-72.
Losen, M., Martinez-Martinez, P., Molenaar, P. C., Lazaridis, K., Tzartos, S., Brenner, T., Duan, R.-S., Luo, J., Lindstrom, J. And Kusner, L., Standardization of the experimental autoimmune myasthenia gravis (EAMG) model by immunization of rats with Torpedo californica acetylcholine receptors—Recommendations for methods and experimental designs. Exp. Neurol. 2015, 270, 18-28.
Riisager A., Duehmke R., Nielsen O. B., Huang C. L. and Pedersen T. H., Determination of cable parameters in skeletal muscle fibres during repetitive firing of action potentials. J Physiol. 2014, 15; 592(20):4417-29.
Elmqvist, D. and Quastel, D. M. J., A quantitative study of end-plate potentials in isolated human muscle. J. Physiol. 1964, 178:505-529.
Osserman, K. E., Kornfeld, P., Cohen, E., Genkins, G., Mendelow, H., Goldberg, H., Windsley, H. and Kaplan, L. I., Studies in myasthenia gravis; review of two hundred eighty-two cases at the Mount Sinai Hospital, New York City, AMA Arch Intern Med., 1958, 102(1):72-81.
Jaretzki, A., Barohn, R. J., Ernstoff, R. M., Kaminski, H. J., Keesey, J. C., Penn, A. S. and Sanders D. B., Myasthenia gravis: recommendations for clinical research standards. Task Force of the Medical Scientific Advisory Board of the Myasthenia Gravis Foundation of America, Neurology, 2000, 55(1):16-23.
Boldingh, M. I., Dekker, L., Maniaol, A. H., Brunborg, C., Lipka, A. F., Niks, E. H., Verschuuren, J. J. G. M. and Tallaksen, C. M. E., An up-date on health-related quality of life in myasthenia gravis-results from population based cohorts, Health Qual. Life Outcomes, 2015, 13:115.
Al-Moallem, M. A., Alkali, N. H., Hakami, M. A. and Zaidan, R. M., Myasthenia gravis: presentation and outcome in 104 patients managed in a single institution, Ann. Saudi Med., 2008; 28(5):341-345.
Besinger, U. A., Toyka, K. V., Homberg, M., Heininger, K., Hohlfeld, R. And Fateh-Moghadam, A., Myasthenia gravis: long-term correlation of binding and bungarotoxin blocking antibodies against acetylcholine receptors with changes in disease severity, Neurology, 1983, 33(10):1316-21.
Tindall, R. S., Rollins, J. A., Phillips, J. T., Greenlee, R. G., Wells, L. and Belendiuk, G., Preliminary results of a double-blind, randomized, placebo-controlled trial of cyclosporine in myasthenia gravis, N. Engl. J. Med., 1987, 316(12):719-24.
Pedersen, T. H., Riisager, A., de Paoli, F. V., Chen, T.-Y. and Nielsen, O. B., Role of physiological CIC-1 Cl⁻ ion channel regulation for the excitability and function of working skeletal muscle, J. Gen. Physiol. 2016, 147:291-308.
Riisager, A. Activity-induced regulation of CIC-1 channels in skeletal muscle and its importance for fibre excitability. (Aarhus University, 2015).
del Castillo, J. and Katz, B., Quantal components of the end-plate potential, J. Physiol. 1954, 124:560-573.
Niks, E. H., Badrising, U. A., Verschuuren, J. J., and van Dijk, J. G., Decremental response of the nasalis and hypothenar muscles in myasthenia gravis, Muscle & Nerve, 2003, 28(2):236-238.
Ruys-Van Oeyen, A. E. W. M. and Van Dijk, J. G., Repetitive nerve stimulation of the nasalis muscle: technique and normal values, Muscle & Nerve, 2002, 26(2):279-282.
Schumm, F., and Stohr, M., Accessory nerve stimulation in the assessment of myasthenia gravis, Muscle & Nerve, 1984, 7(2):147-151.
Katzberg, K. D., Barnett, C., Merkies, I. S J. and Bril, V., Minimal clinically important difference in myasthenia gravis: outcomes from a randomized trial, Muscle & Nerve, 2014, 49(5):661-5.
Burns T M, Conaway M, Sanders D B; M G Composite and MG-QOL15 Study Group. The M G Composite: A valid and reliable outcome measure for myasthenia gravis. Neurology, 2010, 74(18):1434-40.
Diez Porras L, Homedes C, Alberti M A, Velez Santamaria V, Casasnovas C. Quality of Life in Myasthenia Gravis and Correlation of MG-QOL15 with Other Functional Scales, J Clin Med. 2022, 11(8):2189.
Rabin R, de Charro F. EQ-5D: a measure of health status from the EuroQol Group, Ann Med. 2001, 33(5):337-43.
Sanders D B, Arimura K, Cui L, Ertas M, Farrugia M E, Gilchrist J, Kouyoumdjian J A, Padua L, Pitt M, Stålberg E. Guidelines for single fiber EMG, Clin Neurophysiol. 2019, 130(8):1417-1439.
Vincent K A, Carr A J, Walburn J, Scott D L, Rose M R. Construction and validation of a quality of life questionnaire for neuromuscular disease (INQoL), Neurology, 2007, 68(13):1051-7.
Wolfe G I, Herbelin L, Nations S P, Foster B, Bryan W W, Barohn R J. Myasthenia gravis activities of daily living profile, Neurology, 1999, 52(7):1487-9.
Neder J A, Andreoni S, Lerario M C, Nery L E. Reference values for lung function tests. II. Maximal respiratory pressures and voluntary ventilation, Braz J Med Biol Res. 1999, 32(6):719-27.
Oliveira E F, Nacif S R, Urbano J J, Silva A S, Oliveira C S, Perez E A, Polaro M N, Valerio BCO, Stirbulov R, Insalaco G, Oliveira ASB, Oliveira LVF. Sleep, lung function, and quality of life in patients with myasthenia gravis: A cross-sectional study. Neuromuscul Disord. 2017, 27(2):120-127.
Burns™, Conaway M R, Cutter G R, Sanders D B; Muscle Study Group. Less is more, or almost as much: a 15-item quality-of-life instrument for myasthenia gravis, Muscle Nerve, 2008, 38(2):957-63.
Werlauff U, Hojberg A, Firla-Holme R, Steffensen B F, Vissing J. Fatigue in patients with spinal muscular atrophy type II and congenital myopathies: evaluation of the fatigue severity scale, Qual Life Res. 2014, 23(5):1479-88.
Muppidi S, Silvestri N J, Tan R, Riggs K, Leighton T, Phillips G A. Utilization of M G-ADL in myasthenia gravis clinical research and care, Muscle Nerve, 2022, 65(6):630-639.
Jaretzki A 3rd, Barohn R J, Ernstoff R M, Kaminski H J, Keesey J C, Penn A S, Sanders D B. Myasthenia gravis: recommendations for clinical research standards. Task Force of the Medical Scientific Advisory Board of the Myasthenia Gravis Foundation of America. Ann Thorac Surg. 2000 July; 70(1):327-34.
Bedlack R S, Simel D L, Bosworth H, Samsa G, Tucker-Lipscomb B, Sanders D B. Quantitative myasthenia gravis score: assessment of responsiveness and longitudinal validity. Neurology. 2005 Jun. 14; 64(11):1968-70.
Barnett T C, Bril V, Davis A M. Performance of individual items of the quantitative myasthenia gravis score. Neuromuscul Disord. 2013 May; 23(5):413-7.
Barnett C, Katzberg H, Nabavi M, Bril V. The quantitative myasthenia gravis score: comparison with clinical, electrophysiological, and laboratory markers. J Clin Neuromuscul Dis. 2012 June; 13(4):201-5.
Wolfe G I, Barohn R J, Sanders D B, McDermott M P. Comparison of outcome measures from a trial of mycophenolate mofetil in myasthenia gravis. Muscle Nerve. 2008 November; 38(5):1429-1433.
Howard J F Jr, Freimer M, O'Brien F, Wang J J, Collins S R, Kissel J T; MG (Phase2) Study Group. QMG and M G-ADL correlations: Study of eculizumab treatment of myasthenia gravis. Muscle Nerve. 2017 August; 56(2):328-330.
Vissing J, O'Brien F, Wang J J, Howard J F Jr. Correlation between myasthenia gravis-activities of daily living (MG-ADL) and quantitative myasthenia gravis (QMG) assessments of anti-acetylcholine receptor antibody-positive refractory generalized myasthenia gravis in the phase 3 regain study. Muscle Nerve. 2018 August; 58(2):E21-E22.
de Meel RHP, Barnett C, Bril V, Tannemaat M R, Verschuuren J J G M. Myasthenia Gravis Impairment Index: Sensitivity for Change in Generalized Muscle Weakness. J Neuromuscul Dis. 2020; 7(3):297-300.
McPherson T, Aban I, Duda P W, Farzaneh-Far R, Wolfe G I, Kaminski H J, Cutter G, Lee I; of the MGTX Study Group. Correlation of Quantitative Myasthenia Gravis and Myasthenia Gravis Activities of Daily Living scales in the MGTX study. Muscle Nerve. 2020 August; 62(2):261-266.
Howard J F Jr, Utsugisawa K, Benatar M, Murai H, Barohn R J, IIIa I, Jacob S, Vissing J, Burns T M, Kissel J T, Muppidi S, Nowak R J, O'Brien F, Wang J J, Mantegazza R; REGAIN Study Group. Safety and efficacy of eculizumab in anti-acetylcholine receptor antibody-positive refractory generalised myasthenia gravis (REGAIN): a phase 3, randomised, double-blind, placebo-controlled, multicentre study. Lancet Neurol. 2017 December; 16(12):976-986.
Vu T, Meisel A, Mantegazza R, Annane D, Katsuno M, Aguzzi R, Enayetallah A, Beasley K N, Rampal N, Howard J F Jr. for the CHAMPION M G Study Group Terminal complement inhibitor ravulizumab in generalized myasthenia gravis. NEJM Evid. 2022 1(5):1-12.
Howard J F Jr, Nowak R J, Wolfe G I, Freimer M L, Vu T H, Hinton J L, Benatar M, Duda P W, MacDougall J E, Farzaneh-Far R, Kaminski H J; Zilucoplan M G Study Group; Barohn R, Dimachkie M, Pasnoor M, Farmakidis C, Liu T, Colgan S, Benatar M G, Bertorini T, Pillai R, Henegar R, Bromberg M, Gibson S, Janecki T, Freimer M, Elsheikh B, Matisak P, Genge A, Guidon A, David W, Habib A A, Mathew V, Mozaffar T, Hinton J L, Hewitt W, Barnett D, Sullivan P, Ho D, Howard J F Jr, Traub R E, Chopra M, Kaminski H J, Aly R, Bayat E, Abu-Rub M, Khan S, Lange D, Holzberg S, Khatri B, Lindman E, Olapo T, Sershon L M, Lisak R P, Bernitsas E, Jia K, Malik R, Lewis-Collins T D, Nicolle M, Nowak R J, Sharma A, Roy B, Nye J, Pulley M, Berger A, Shabbir Y, Sachdev A, Patterson K, Siddiqi Z, Sivak M, Bratton J, Small G, Kohli A, Fetter M, Vu T, Lam L, Harvey B, Wolfe G I, Silvestri N, Patrick K, Zakalik K, Duda P W, MacDougall J, Farzaneh-Far R, Pontius A, Hoarty M. Clinical Effects of the Self-administered Subcutaneous Complement Inhibitor Zilucoplan in Patients With Moderate to Severe Generalized Myasthenia Gravis: Results of a Phase 2 Randomized, Double-Blind, Placebo-Controlled, Multicenter Clinical Trial. JAMA Neurol. 2020 May 1; 77(5):582-592.
Bril V, Benatar M, Andersen H, Vissing J, Brock M, Greve B, Kiessling P, Woltering F, Griffin L, Van den Bergh P; MG0002 Investigators. Efficacy and Safety of Rozanolixizumab in Moderate to Severe Generalized Myasthenia Gravis: A Phase 2 Randomized Control Trial. Neurology. 2021 Feb. 9; 96(6):e853-e865.
Howard J F Jr, Bril V, Vu T, Karam C, Peric S, Margania T, Murai H, Bilinska M, Shakarishvili R, Smilowski M, Guglietta A, Ulrichts P, Vangeneugden T, Utsugisawa K, Verschuuren J, Mantegazza R; ADAPT Investigator Study Group. Safety, efficacy, and tolerability of efgartigimod in patients with generalised myasthenia gravis (ADAPT): a multicentre, randomised, placebo-controlled, phase 3 trial. Lancet Neurol. 2021 July; 20(7):526-536.

1. A method for treating myasthenia gravis in a subject comprising administering a composition comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition contains the (2S)-2-[4-bromo-2-(1,2-oxazol-3-yl)phenoxy]propanoic acid, or the pharmaceutically acceptable salt, hydrate, polymorph, tautomer, or solvate thereof in a therapeutic dose of 100 to 1500 mg.

2. The method according to claim 1, wherein the therapeutic dose is 200 to 600 mg.

3. The method according to claim 1, wherein the composition is administered two times daily.

4. The method according to claim 1, wherein the composition is administered orally.

5. The method according to claim 1, wherein the composition is a solid dosage form.

6. The method according to claim 1, wherein the composition is dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where Tₘₐₓ is reached within 1 to 5 hours after administration.

7. The method according to claim 1, wherein the composition is dosed orally using a solid dosage form and provides a plasma concentration-time profile of (2S)-2-[4-bromo-2-(1,2-oxazol-3-yl)phenoxy]propanoic acid, or a pharmaceutically acceptable salt, hydrate, polymorph, tautomer, or solvate thereof, where the mean Cₘₐₓ is 13,000 to 32,000 ng/mL after administration with a single dose of 400 mg (2S)-2-[4-bromo-2-(1,2-oxazol-3-yl)phenoxy]propanoic acid, or a pharmaceutically acceptable salt, hydrate, polymorph, tautomer, or solvate thereof.

8. The method according to claim 1, wherein a Quantitative Myasthenia Gravis (QMG) total score is reduced compared to placebo at the same time point after treatment and the reduction in QMG total score after treatment is at least 0.9 points.

9. The method according to claim 1, wherein the subject is suffering from ocular myasthenia gravis, early-onset generalised myasthenia gravis, late-onset generalised myasthenia gravis, generalized myasthenia gravis, seropositive myasthenia gravis, seronegative myasthenia gravis, AChR antibody positive myasthenia gravis, or muscle-specific kinase antibody-positive myasthenia gravis (MuSK-MG).

10. A composition, formulated as a solid dosage form, comprising (2S)-2-[4-bromo-2-(1,2-oxazol-3-yl)phenoxy]propanoic acid, or a pharmaceutically acceptable salt, hydrate, polymorph, tautomer, or solvate thereof, wherein the composition comprises 50 to 400 mg (2S)-2-[4-bromo-2-(1,2-oxazol-3-yl)phenoxy]propanoic acid.

11. The composition according to claim 10, wherein the composition comprises 10 to 80 wt % (2S)-2-[4-bromo-2-(1,2-oxazol-3-yl)phenoxy]propanoic acid, or a pharmaceutically acceptable salt, hydrate, polymorph, tautomer, or solvate thereof.

12. The composition according to claim 10, wherein the composition further comprises at least one pharmaceutically acceptable adjuvant and/or excipient selected from the group consisting of filler, binder, lubricant and disintegrant.

13. The composition according to claim 10, wherein the composition comprises:
a. 10 to 80 wt %, such as 40 to 65 wt %, such as 50 to 60 wt %, such as 50 to 55 wt %, such as 55 to 60 wt %, such as about 53 wt %, such as about 56 wt % (2S)-2-[4-bromo-2-(1,2-oxazol-3-yl)phenoxy]propanoic acid, or a pharmaceutically acceptable salt, hydrate, polymorph, tautomer, or solvate thereof;
b. 5 to 60 wt %, such as 20 to 40 wt %, such as 21 to 37 wt % silicified microcrystalline cellulose;
c. 2 to 60 wt %, such as 5 to 16 wt % microcrystalline cellulose;
d. 1 to 15 wt %, such as 1.5 to 7 wt %, such as 1.8 to 6.0 wt % maltodextrin;
e. 0.25 to 3 wt %, such as 0.4 to 2.0 wt % magnesium stearate; and
f. 0.25 to 5 wt %, such as 0.3 to 2.5 wt % Croscarmellose sodium;
with the proviso that the sum of the wt % of the components does not exceed 100 wt %

14. A kit-of-parts or a composition comprising (2S)-2-[4-bromo-2-(1,2-oxazol-3-yl)phenoxy]propanoic acid, or a pharmaceutically acceptable salt, hydrate, polymorph, tautomer, or solvate thereof, and an acetylcholinesterase inhibitor for use in a method for treatment of myasthenia gravis in a subject.

15. The kit-of-parts for use according to claim 14, wherein (2S)-2-[4-bromo-2-(1,2-oxazol-3-yl)phenoxy]propanoic acid, or a pharmaceutically acceptable salt, hydrate, polymorph, tautomer, or solvate thereof, is present at a therapeutic dose of 100 to 1500 mg.

16. A method for reducing inhibitory effects at neuromuscular junctions in a subject, the method comprising:
administering to the subject a composition including a therapeutic dose of 100 to 600 mg of (2S)-2-[4-bromo-2-(1,2-oxazol-3-yl)phenoxy]propanoic acid, or a pharmaceutically acceptable salt, hydrate, polymorph, tautomer, or solvate thereof,
wherein the composition is administered once, twice or three times daily and is in a solid dosage form, and
wherein the administration of the therapeutic dose reduces jitter and/or blocking in the subject.

17. The method according to claim 16, wherein administration of the therapeutic dose reduces jitter in the subject by at least 10%.

18. The method according to claim 16, wherein administration of the therapeutic dose reduces jitter in the subject by 5% to 95%.

19. The method according to claim 16, wherein administration of the therapeutic dose reduces jitter in the subject by at least 5 μs.

20. The method according to 16, wherein administration of the therapeutic dose reduces jitter in the subject by 5 μs to 200 μs.

21. The method according to claim 16, wherein administration of the therapeutic dose reduces blocking in the subject by at least 5%.

22. The method according to claim 16, wherein administration of the therapeutic dose reduces blocking in the subject by 5% to 95%.
